SEQUENCE LISTING (8 sequences)

SEQ ID NO: 1 - DNA, 29996 bp, Artificial Sequence
  Synthetic primer
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatcgacctgcaggcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgatgttgaaagaatatataacactgacgatgagctcaacaatgcaggaagcgacgcaccgaaggaggaagagcctggtgattgaaggaccatcgaagacgggtaaaacgcaatgggctatatcattgggaatacataactactggtgtgaatcagtagacttcagtctatacatagacgacgctttgtacaatattattgacgatataccattccaatatttgccttgtaaaaaagccttattaggttgtcaattcaattacattgcaaatgaaaaatatcgtccaaagcggaaaatcaaaggagggatcccatcaattgttctttgtaatccagataactcttactatgatgcaatcaccaattggcatgcaacattcaagccgtgggctgaagaaaacattatctttgtaaaaattgatagaccgttgtactgatcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgtcagtacaacggtctatcaatttttacaaagataatgttttcttcagcccacggcttgaatgttgcatgccaattggtgattgcatcatagtaagagttatctggattacaaagaacaattgatgggatccctcctttgattttccgctttggacgatatttttcatttgcaatgtaattgaattgacaacctaataaggcttttttacaaggcaaatattggaatggtatatcgtcaataatattgtacaaagcgtcgtctatgtatagactgaagtctactgattcacaccagtagttatgtattcccaatgatatagcccattgcgttttacccgtcttcgatggtccttcaatcaccaggctcttcctccttcggtgcgtcgcttcctgcattgttgagctcatcgtcagtgttatatattctttcaacatctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatggcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaattcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatcgacctgcaggcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgatgttgaaagaatatataacactgacgatgagctcaacaatgcaggaagcgacgcaccgaaggaggaagagcctggtgattgaaggaccatcgaagacgggtaaaacgcaatgggctatatcattgggaatacataactactggtgtgaatcagtagacttcagtctatacatagacgacgctttgtacaatattattgacgatataccattccaatatttgccttgtaaaaaagccttattaggttgtcaattcaattacattgcaaatgaaaaatatcgtccaaagcggaaaatcaaaggagggatcccatcaattgttctttgtaatccagataactcttactatgatgcaatcaccaattggcatgcaacattcaagccgtgggctgaagaaaacattatctttgtaaaaattgatagaccgttgtactgatcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgtcagtacaacggtctatcaatttttacaaagataatgttttcttcagcccacggcttgaatgttgcatgccaattggtgattgcatcatagtaagagttatctggattacaaagaacaattgatgggatccctcctttgattttccgctttggacgatatttttcatttgcaatgtaattgaattgacaacctaataaggcttttttacaaggcaaatattggaatggtatatcgtcaataatattgtacaaagcgtcgtctatgtatagactgaagtctactgattcacaccagtagttatgtattcccaatgatatagcccattgcgttttacccgtcttcgatggtccttcaatcaccaggctcttcctccttcggtgcgtcgcttcctgcattgttgagctcatcgtcagtgttatatattctttcaacatctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatggcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaaggg

SEQ ID NO: 2 - DNA, 15509 bp, Artificial Sequence
  Synthetic primer
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgatggtaacactgaacaaacggaatcgcgttcttcctgagtgcgattcctgcagttctagtgaaagttctttgaatgatattgatatttgtggtgatgatgatgggttaggggatgaggctttagacgctggatccgtttattcgtcgtcacagaaactgttagtttctgtggctaaagatgttcttttagatgactgtgattcaacgatattggatatatcgttgccttctgctttatggtttttgtcgcaaagatatttgacttgttgtttgaggaaagaattactgcctctgccaggtatatccgagaaacagactgttttattgcgacagctgattaggcgtgtcgctcgtcgtcattgtttatttacttacaagtgcgaggagtggtttgagggttgtttgaagataaagaaggatggtaatgaaaaaaaggagccgccaacggaagcagagaagaaggcgcaggacgactgggaggagttctgccgtaaggcggcgtgctcggcctcgtagtcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgctacgaggccgagcacgccgccttacggcagaactcctcccagtcgtcctgcgccttcttctctgcttccgttggcggctcctttttttcattaccatccttctttatcttcaaacaaccctcaaaccactcctcgcacttgtaagtaaataaacaatgacgacgagcgacacgcctaatcagctgtcgcaataaaacagtctgtttctcggatatacctggcagaggcagtaattctttcctcaaacaacaagtcaaatatctttgcgacaaaaaccataaagcagaaggcaacgatatatccaatatcgttgaatcacagtcatctaaaagaacatctttagccacagaaactaacagtttctgtgacgacgaataaacggatccagcgtctaaagcctcatcccctaacccatcatcatcaccacaaatatcaatatcattcaaagaactttcactagaactgcaggaatcgcactcaggaagaacgcgattccgtttgttcagtgttaccatctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatggcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaaggg

SEQ ID NO: 3 - DNA, 7082 bp, Artificial Sequence
  Synthetic primer
tcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgatgttgaaagaatatataacactgacgatgagctcaacaatgcaggaagcgacgcaccgaaggaggaagagcctggtgattgaaggaccatcgaagacgggtaaaacgcaatgggctatatcattgggaatacataactactggtgtgaatcagtagacttcagtctatacatagacgacgctttgtacaatattattgacgatataccattccaatatttgccttgtaaaaaagccttattaggttgtcaattcaattacattgcaaatgaaaaatatcgtccaaagcggaaaatcaaaggagggatcccatcaattgttctttgtaatccagataactcttactatgatgcaatcaccaattggcatgcaacattcaagccgtgggctgaagaaaacattatctttgtaaaaattgatagaccgttgtactgatcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgtcagtacaacggtctatcaatttttacaaagataatgttttcttcagcccacggcttgaatgttgcatgccaattggtgattgcatcatagtaagagttatctggattacaaagaacaattgatgggatccctcctttgattttccgctttggacgatatttttcatttgcaatgtaattgaattgacaacctaataaggcttttttacaaggcaaatattggaatggtatatcgtcaataatattgtacaaagcgtcgtctatgtatagactgaagtctactgattcacaccagtagttatgtattcccaatgatatagcccattgcgttttacccgtcttcgatggtccttcaatcaccaggctcttcctccttcggtgcgtcgcttcctgcattgttgagctcatcgtcagtgttatatattctttcaacatctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctatcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgatgttgaaagaatatataacactgacgatgagctcaacaatgcaggaagcgacgcaccgaaggaggaagagcctggtgattgaaggaccatcgaagacgggtaaaacgcaatgggctatatcattgggaatacataactactggtgtgaatcagtagacttcagtctatacatagacgacgctttgtacaatattattgacgatataccattccaatatttgccttgtaaaaaagccttattaggttgtcaattcaattacattgcaaatgaaaaatatcgtccaaagcggaaaatcaaaggagggatcccatcaattgttctttgtaatccagataactcttactatgatgcaatcaccaattggcatgcaacattcaagccgtgggctgaagaaaacattatctttgtaaaaattgatagaccgttgtactgatcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgtcagtacaacggtctatcaatttttacaaagataatgttttcttcagcccacggcttgaatgttgcatgccaattggtgattgcatcatagtaagagttatctggattacaaagaacaattgatgggatccctcctttgattttccgctttggacgatatttttcatttgcaatgtaattgaattgacaacctaataaggcttttttacaaggcaaatattggaatggtatatcgtcaataatattgtacaaagcgtcgtctatgtatagactgaagtctactgattcacaccagtagttatgtattcccaatgatatagcccattgcgttttacccgtcttcgatggtccttcaatcaccaggctcttcctccttcggtgcgtcgcttcctgcattgttgagctcatcgtcagtgttatatattctttcaacatctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaat

SEQ ID NO: 4 - DNA, 435 bp, Artificial Sequence
  Synthetic primer
atgttgaaagaatatataacactgacgatgagctcaacaatgcaggaagcgacgcaccgaaggaggaagagcctggtgattgaaggaccatcgaagacgggtaaaacgcaatgggctatatcattgggaatacataactactggtgtgaatcagtagacttcagtctatacatagacgacgctttgtacaatattattgacgatataccattccaatatttgccttgtaaaaaagccttattaggttgtcaattcaattacattgcaaatgaaaaatatcgtccaaagcggaaaatcaaaggagggatcccatcaattgttctttgtaatccagataactcttactatgatgcaatcaccaattggcatgcaacattcaagccgtgggctgaagaaaacattatctttgtaaaaattgatagaccgttgtactga

SEQ ID NO: 5 - DNA, 435 bp, Artificial Sequence
  Synthetic primer
tcagtacaacggtctatcaatttttacaaagataatgttttcttcagcccacggcttgaatgttgcatgccaattggtgattgcatcatagtaagagttatctggattacaaagaacaattgatgggatccctcctttgattttccgctttggacgatatttttcatttgcaatgtaattgaattgacaacctaataaggcttttttacaaggcaaatattggaatggtatatcgtcaataatattgtacaaagcgtcgtctatgtatagactgaagtctactgattcacaccagtagttatgtattcccaatgatatagcccattgcgttttacccgtcttcgatggtccttcaatcaccaggctcttcctccttcggtgcgtcgcttcctgcattgttgagctcatcgtcagtgttatatattctttcaacat

SEQ ID NO: 6 - DNA, 3886 bp, Artificial Sequence
  Synthetic primer
tcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgatggtaacactgaacaaacggaatcgcgttcttcctgagtgcgattcctgcagttctagtgaaagttctttgaatgatattgatatttgtggtgatgatgatgggttaggggatgaggctttagacgctggatccgtttattcgtcgtcacagaaactgttagtttctgtggctaaagatgttcttttagatgactgtgattcaacgatattggatatatcgttgccttctgctttatggtttttgtcgcaaagatatttgacttgttgtttgaggaaagaattactgcctctgccaggtatatccgagaaacagactgttttattgcgacagctgattaggcgtgtcgctcgtcgtcattgtttatttacttacaagtgcgaggagtggtttgagggttgtttgaagataaagaaggatggtaatgaaaaaaaggagccgccaacggaagcagagaagaaggcgcaggacgactgggaggagttctgccgtaaggcggcgtgctcggcctcgtagtcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgctacgaggccgagcacgccgccttacggcagaactcctcccagtcgtcctgcgccttcttctctgcttccgttggcggctcctttttttcattaccatccttctttatcttcaaacaaccctcaaaccactcctcgcacttgtaagtaaataaacaatgacgacgagcgacacgcctaatcagctgtcgcaataaaacagtctgtttctcggatatacctggcagaggcagtaattctttcctcaaacaacaagtcaaatatctttgcgacaaaaaccataaagcagaaggcaacgatatatccaatatcgttgaatcacagtcatctaaaagaacatctttagccacagaaactaacagtttctgtgacgacgaataaacggatccagcgtctaaagcctcatcccctaacccatcatcatcaccacaaatatcaatatcattcaaagaactttcactagaactgcaggaatcgcactcaggaagaacgcgattccgtttgttcagtgttaccatctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaat

SEQ ID NO: 7 - DNA, 516 bp, Artificial Sequence
  Synthetic primer
atggtaacactgaacaaacggaatcgcgttcttcctgagtgcgattcctgcagttctagtgaaagttctttgaatgatattgatatttgtggtgatgatgatgggttaggggatgaggctttagacgctggatccgtttattcgtcgtcacagaaactgttagtttctgtggctaaagatgttcttttagatgactgtgattcaacgatattggatatatcgttgccttctgctttatggtttttgtcgcaaagatatttgacttgttgtttgaggaaagaattactgcctctgccaggtatatccgagaaacagactgttttattgcgacagctgattaggcgtgtcgctcgtcgtcattgtttatttacttacaagtgcgaggagtggtttgagggttgtttgaagataaagaaggatggtaatgaaaaaaaggagccgccaacggaagcagagaagaaggcgcaggacgactgggaggagttctgccgtaaggcggcgtgctcggcctcgtag

SEQ ID NO: 8 - DNA, 516 bp, Artificial Sequence
  Synthetic primer
ctacgaggccgagcacgccgccttacggcagaactcctcccagtcgtcctgcgccttcttctctgcttccgttggcggctcctttttttcattaccatccttctttatcttcaaacaaccctcaaaccactcctcgcacttgtaagtaaataaacaatgacgacgagcgacacgcctaatcagctgtcgcaataaaacagtctgtttctcggatatacctggcagaggcagtaattctttcctcaaacaacaagtcaaatatctttgcgacaaaaaccataaagcagaaggcaacgatatatccaatatcgttgaatcacagtcatctaaaagaacatctttagccacagaaactaacagtttctgtgacgacgaataaacggatccagcgtctaaagcctcatcccctaacccatcatcatcaccacaaatatcaatatcattcaaagaactttcactagaactgcaggaatcgcactcaggaagaacgcgattccgtttgttcagtgttaccat

(19) United States
(12) Patent Application Publication
(10) Pub. No.: US 2023/0047498 A1
(43) Pub. Date: Feb. 16, 2023

(54) METHOD AND COMPOSITIONS FOR ENGINEERING GRAPEVINE RED BLOTCH VIRUS-RESISTANT GRAPEVINE

(71) Applicant: Texas Tech University System, Lubbock, TX (US)

(72) Inventors: Chris Rock, Lubbock, TX (US); Sunitha Sukumaran, Lubbock, TX (US)

(21) Appl. No.: 17/782,768

(22) PCT Filed: Dec. 9, 2020
(86) PCT No.: PCT/US2020/064014

§ 371 Date: Jun. 6, 2022

Related U.S. Application Data
(60) Provisional application No. 62/945,356, filed on Dec. 9, 2019.

(51) Int. Cl. C12N 15/82

(52) U.S. Cl. CPC C12N 15/8283; C12N 15/8218; C12N 15/8213

(57) ABSTRACT

Compositions and methods for transforming cells and generating transgenic grapevine plants comprising two silencing suppressor proteins C2 and V2 encoded by GRBV and recombinant hairpin vectors targeting C2 and V2, which are be used to generate stably transformed transgenic grapevine plants.

### CROSS-REFERENCE TO RELATED APPLICATIONS

This application claims priority to U.S. Provisional Application Ser. No. 62/945,356, filed Dec. 9, 2019, the entire contents of which are incorporated herein by reference.

### TECHNICAL FIELD OF THE INVENTION

The present invention relates in general to the field of genetic alteration of plants, and more particularly, to methods and compositions for engineering Grapevine Red Blotch Virus-resistant grapevines.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Dec. 9, 2020, is named TECH2146WO_SeqList.txt and is 76,kilo bytes in size.

### BACKGROUND OF THE INVENTION

Nearly 90 different documented viruses from 17 families infect grapes worldwide, which is far greater than the number of viruses documented in any other single perennial crop [3, 4]. Geminiviruses are single-stranded (ss) DNA viruses that cause major losses to many crops throughout the world [5]. Geminiviridae constitutes the second largest family of plant viruses. Geminiviruses are characterized by small, circular, ssDNA genomes encapsidated in twinned (hence, the name Gemini) icosahedral particles. They are insect vector-transmissible and infect both monocotyledonous and dicotyledonous plants [6]. The genomes are either monopartite or bipartite with circular DNA molecules of 2.5-3 kilobases. Geminiviruses possess a highly conserved core region (CR) of ˜200 nucleotides containing bidirectional tissue-specific promoters and an inverted repeat that forms a hairpin loop with an invariant 9-nt 5′-TAATATT-AC-3′ that acts as the origin of Virion (V) strand DNA replication in host (and some vectors), and is the target of DNA methylation, an epigenetic transcriptional silencing mechanism for host immune response. The viral gene products are required for replication and transmission [7]. The geminiviral proteins perform multiple functions at every stage of the viral life cycle such as transport, insect-mediated transmission, and manipulation of host innate immune responses. They impede the host's multi-layered antiviral mechanisms including post-transcriptional and transcriptional gene silencing, and the salicylic acid-mediated hypersensitive response [8].

Grapevine Red Blotch Virus (GRBV) is a monopartite, grapevine-infecting Grablovirus first observed in California in 2008 as associated with Red Blotch Disease [1, 9] and later proven by fulfilled Koch's postulates, including by grafting and vegetative propagation as primary inoculum, to be the causal agent of Red Blotch [2, 10]. The V1 protein load is found to be six times higher in petioles compared to leaves, which supports the notion that GRBV is phloem-restricted or phloem-limited [11]. Disease symptoms manifest as red patches in the middle of the grapevine leaf and in veins and petiole, which coalesce at the end of the growing season similar to leafroll viral infections and potassium or phosphorous deficiencies [12, 13]. Infected white-berried V. vinifera cultivars show chlorosis and cupping, similar to leafroll virus or magnesium deficiency. Similar to other grapevine viruses, infection of GRBV in rootstocks is latent [10]. GRBV infection results in lower pruning mass and less winter hardiness of buds, reduced photosynthesis and stomatal conductance of leaves, delayed and uneven berry ripening, higher titratable acids, and reduced sugar, tannin, and anthocyanin contents in the berry [14, 15]. The impact of GRBV on foliar physiology is higher glucose and fructose, higher phenolics and terpenoids, and an altered amino acid profile [16]. Consequences of infections are reduced carbon translocation and impairment of fruit qualities for both table grape [17] and wine industries such as less alcohol, color, flavor, and aroma and increased astringency, flavonol, proanthocyanidin, and aftertaste of vegetal character [18-20], with estimated price/quality penalties for vineyard producers as high as $68,000/ha [21]. Drought stress of grapevines during ripening can improve fruit properties including anthocyanins and skin tannins, but not in GRBV-infected vines [22].

GRBV was initially detected in ˜95% of symptomatic grapevines and in ˜2.7% of asymptomatic grapevines [1]. Highest virus titers are found in the petioles of fully expanded leaves but significantly reduced levels of virus in the shoot extremities [23]. Limited genetic diversity of GRBV populations in newly infected vines supports localized secondary spread within and between vineyards of 1-2% per year by a flying insect [24-26]. At Jacksonville in southern Oregon, 3% of vines were infected with GRBV in 2014, and GRBV incidence reached 58% of spatially associated study vines by 2016 [27]. Bander et al. [28] identified the three-cornered alfalfa treehopper Spissistilus festinus as the candidate vector that transmits GRBV under laboratory conditions, whereas Poojari et al. [29] claimed Virginia creeper leafhopper (Erythroneura ziczac (Walsh)), a dominant invasive species of northern California vineyards since the 1980s as the candidate vector (http://www.ucanr.org/blogs/blogcore/postdetail.cfm?postnum=38818). Cover crop and arthropod samples collected from GRBV-infected California vineyards with emphasis on legume species (preferred host of S. festinus) did not correlate for GRBV, suggesting a minimal role, if any, for cover crops as secondary inoculum reservoirs [24]. Other hemipteran species from vineyard traps testing positive for GRBV as candidate vectors are Colladonus reductus (Cicadellidae), Osbornellus borealis (Cicadellidae), and a Melanoliarus species (Cixiidae), but to date only S. festinus has evidences of significant spatial distributions and phylogenic analysis of GRBV sequences associated with infected vines [28, 30]. Cultivating non-legume cover crops like fescue or California poppy that do not support S. festinus survival or oviposition may reduce vector establishment in vineyards [32].

What is needed are novel methods and compositions for the genetic alteration of plants, and more particularly, to methods and compositions for engineering Grapevine Red Blotch Virus-resistant grapevines.

### SUMMARY OF THE INVENTION

In one embodiment, the present invention includes a transformed or transgenic plant that is resistant to a Grapevine Red Blotch Virus (GRBV), wherein the transformed or transgenic plant comprises: at least one nucleic acid construct comprising: a recombinant nucleic acid sequence encoding a suppressor of expression of a C2, a V2, or both proteins, of the GRBV, wherein when the construct reduces the expression of the C2, V2, or both proteins in a plant that regulates transcription or expression of the C2, V2, or both proteins and confers resistance to the GRBV in the plant as compared to a control plant. In one aspect, the plant is a grapevine. In another aspect, expression of the suppressor is regulated by a constitutive, inducible, or tissue-enhanced promoter. In another aspect, expression of the suppressor is regulated by a 35S promoter. In another aspect, the suppressor is a gene silencing nucleic acid that is, or is derived from, a small RNA (sRNA), microRNA (miRNA), short hairpin RNA (shRNA), bifunctional shRNA, clustered regularly interspaced palindromic repeats (CRISPR) guide RNA, or small interfering RNA (siRNA). In another aspect, a transformed plant cell is an embryogenic cell in globular state. In another aspect, the plant comprises one or more transformed or transgenic plant cells, and the transformed or transgenic plant cell is a grapevine cell. In another aspect, a transformed or transgenic plant cell is a cell of one of the following grapevine varieties: 101-14 Mgt, 110 Richter, 1103 Paulson, Freedom or Harmony. In another aspect, a transformed plant cell is a cell of the 101-14 grapevine variety. In another aspect, the suppressor has at least 90, 91, 92, 93, 94, 95, 96, 97, 98, 99%, or 100% percent sequence identity with at least one of SEQ ID NOS: 3, 4, 5, 6, 7, or 8.

In another embodiment, the present invention includes a plant part or plant material derived from a transformed or transgenic plant that is resistant to a Grapevine Red Blotch Virus (GRBV), wherein the transformed or transgenic plant comprises: at least one nucleic acid construct comprising: a recombinant nucleic acid sequence encoding a suppressor of expression of a C2, a V2, or both proteins, of the GRBV, wherein when the construct reduces the expression of the C2, V2, or both proteins in a plant that regulates transcription or expression of the C2, V2, or both proteins and confers resistance to the GRBV in the plant as compared to a control plant.

In one embodiment, the present invention includes a method of producing a Grapevine Red Blotch Virus (GRBV) resistant transgenic grapevine plant, wherein the method comprises introducing at least one nucleic acid construct comprising: a recombinant nucleic acid sequence encoding a suppressor of expression of a C2, a V2, or both proteins, of the GRBV, wherein when the construct reduces the expression of the C2, V2, or both proteins in a plant, wherein the suppressor regulates transcription or expression of the C2, V2, or both proteins and confers resistance to GRBV in the plant as compared to a control plant.

In another embodiment, the present invention includes a Grapevine Red Blotch Virus (GRBV) resistant grapevine plant produced by the method above.

In one embodiment, the present invention includes a recombinant DNA vector plasmid that confers resistance against a Grapevine Red Blotch Virus (GRBV), wherein the vector plasmid contains one or more gene silencing nucleic acids against SEQ ID NO: 1, 2, or both. In one aspect, the vector plasmid further contains a gene conferring antibiotic resistance. In another aspect, the vector plasmid comprises a neomycin phosphotransferase II (nptII) gene conferring kanamycin resistance.

In another embodiment, the present invention includes a transformed plant cell wherein the transformed plant cell contains and expresses one or more of the gene silencing nucleic acids in the vector plasmid of the plant above.

In another embodiment, the present invention includes a method to confer resistance against a Grapevine Red Blotch Virus (GRBV) in non-transgenic grapevines, wherein the method comprises the steps of: providing a group of plant cells transformed with a vector plasmid comprising: a recombinant nucleic acid sequence encoding a suppressor of the expression of a C2, a V2, or both proteins, of the GRBV, wherein when the construct reduces transcription or expression of the proteins C2, V2, or both proteins, and confers resistance to GRBV in the plant as compared to a control plant; culturing the group of transformed plant cells to form transgenic seedlings resistant to the GRBV; culturing the transgenic seedlings to take roots; cutting an aerial part of the transgenic seedlings; grafting a non-transgenic grapevine woody graft onto the seedling; and culturing the graft wherein the non-transgenic grapevine plant acquires resistance against the GRBV from phloem transport of the transgenic plant. Endogenous siRNAs can pass through plasmodesmata and move across graft unions in phloem to regulate gene expression by epigenetic modifications, establishing developmental gradients, or by feedback loops in adjacent cells or in separate roots and shoots [155-158]. In another aspect, the suppressor is a gene silencing nucleic acid that reduces the transcription or expression of the C2, V2, or both proteins of the GRBV. In another aspect, the grafted non-transgenic grapevine is Vitis vinifera. In one aspect, the grafted non-transgenic grapevine is a variety of Vitis vinifera selected among the following table grape varieties: Autumn royal, Black seedless, Calmeria, Emperor, Flame seedless, Loose Perlette, Red Malaga, Ruby seedless, Loose Perlette, Thompson seedless, Red Globe, Sugarone and Superior seedless. In another aspect, the grafted non-transgenic grapevine is a variety of Vitis vinifera selected among the following wine grape varieties: Carmenere, Cabernet sauvignon, Cabernet Franc, Syrah, Chardonnay, Chenin, Colombard, Courdec, Dattier, Emerald, Gamay, Grenache, Malbec, Merlot, Mission, Muscat, Petit Verdot, Pinot noir, Riesling, Sauvignon, Sauvignon blanc, Semillon, Shiraz, Tempranillo, Zinfandel. In another aspect, the suppressor has at least 90, 91, 92, 93, 94, 95, 96, 97, 98, 99%, or 100% percent sequence identity with at least one of SEQ ID NOS: 3, 4, 5, 6, 7, or 8.

In another embodiment, the present invention includes a Grapevine Red Blotch Virus (GRBV) resistant grapevine plant produced by the method above.

In another embodiment, the present invention includes a method for producing a grapevine plant resistant to a Grapevine Red Blotch Virus (GRBV), the method comprising crossing two grapevine plants, harvesting the resultant seed or embryo and growing the seed or embryo into a mature grapevine plant, wherein at least one grapevine plant is the grapevine plant described above. In another aspect, one of the grapevine plants is transgenic and the other is a non-transgenic grapevine of Vitis vinifera selected among the following table grape varieties: Autumn royal, Black seedless, Calmeria, Emperor, Flame seedless, Loose Perlette, Red Malaga, Ruby seedless, Loose Perlette, Thompson seedless, Red Globe, Sugarone and Superior seedless.

In another embodiment, the present invention includes a grapevine plant or plant part of a transgenic grapevine cultivar, or wherein a representative sample of the plant was deposited under NCIMB No. ______.

In another embodiment, the present invention includes a grapevine plant, or a part thereof, produced by growing the deposited sample of the plant described above.

In another embodiment, the present invention includes a grapevine plant, or a part thereof, clonally propagated from the plant of the plant described above.

In another embodiment, the present invention includes a tissue culture of cells produced from protoplasts or cells from the plant of the plant above, wherein the cells or protoplasts are produced from a plant part selected from the group consisting of leaf, pollen, embryo, cotyledon, hypocotyl, meristematic cell, root, root tip, pistil, anther, flowers, stem and fruit.

In another embodiment, the present invention includes a grapevine plant regenerated from the tissue culture of the plant described above, wherein the plant is resistant to the GRBV.

### BRIEF DESCRIPTION OF THE DRAWINGS

The foregoing and other objects, features, and advantages of the present invention will be apparent from the following description of embodiments as illustrated in the accompanying drawings, in which reference characters refer to the same parts throughout the various views. The drawings are not necessarily to scale, emphasis instead being placed upon illustrating principles of the present invention.

FIG. 1. Genome Organization of Grapevine red blotch virus (GRBV), previously called Grapevine red blotch-associated virus (GRBaV). The core region (CR) of ˜200 nucleotides contains bidirectional tissue-specific promoters and an inverted repeat that forms a hairpin loop with an invariant 9-nt 5′-TAATATT↓AC-3′ that is nicked by the viral replicase and acts as the origin of Virion (V) strand DNA replication in host. Modified from [33].

FIG. 2. Agroinfiltration of the Nicotiana benthamiana 16c-GFP to demonstrate silencing suppression by HcPro and test GRBV genes. Agrobacterium-mediated transient expression in N. benthamiana leaves. IM: Mock-agroinfiltrated with infiltration medium. mgfp: Agroinfiltrated with the A. tumefaciens strain harboring P35S-gfp) as control for GFP transgene silencing. mgfp+pCAM-2301: Co-agroinfiltrated with strains harboring P35S-gfp+pCAMBIA-2301 (negative control). mgfp+HcPro: Co-agroinfiltrated with strains harboring P35S-gfp+Potyvirus HcPro (Positive control for PTGS). mgfp+C1: Co-agroinfiltrated with strains harboring P35S-gfp+GRBV C1. mgfp+C2: Co-agroinfiltrated with strains harboring P35S-gfp+GRBV C2; Note PTGS suppression evidence, like HcPro. mgfp+C3: Co-agroinfiltrated with strains harboring P35S-gfp+GRBV C3. mgfp+V1: Co-agroinfiltrated with strains harboring P35S-gfp+GRBV V1. mgfp+V2: Co-agroinfiltrated with strains harboring P35S-gfp+GRBV V2. Note PTGS suppression evidence, like HcPro. mgfp+V3: Co-agroinfiltrated with strains harboring P35S-gfp+GRBV V3. Red chlorophyll autofluorescence and green GFP fluorescence of leaves were visualized with hand-held UV-B light source.

FIGS. 3A and 3B show the validation of silencing suppression by GRBV genes C2 and V2. (FIG. 3A) Agrobacterium-mediated transient expression in N. benthamiana leaves. IM: Mock-agroinfiltrated with infiltration medium. mgfp: Agroinfiltrated with the A. tumefaciens strain harboring P35S-gfp as control for GFP transgene silencing. mgfp+pCAM-2301: Co-agroinfiltrated with strains harboring P35S-gfp+pCAMBIA-2301 (negative control). mgfp+HcPro: Co-agroinfiltrated with strains harboring P35S-gfp+Potyvirus HcPro (Positive control for PTGS). mgfp+C2: Co-agroinfiltrated with strains harboring P35S-gfp+GRBV C2; mgfp+V2: Co-agroinfiltrated with strains harboring P35S-gfp+GRBV V2; mgfp+C2+V2: Co-agroinfiltrated with strains harboring P35S-gfp+GRBV C2+GRBV V2; (FIG. 3B) Northern blot analysis with gfp probe. Total RNA from the infiltrated areas of mock infiltrated leaves (IM), P35S-gfp infiltrated leaves (mgfp), P35Sgfp+C2 (mgfp+C2) P35Sgfp+V2 (mgfp+V2) and P35Sgfp+HcPro (mgfp+HcPro), P35Sgfp+C2+V2 (mgfp+C2+V2) was probed with the gfp coding sequence. The 18S rRNA portion of the ethidium bromide-stained gel is placed at the bottom as control for RNA loadings. Red chlorophyll autofluorescence and green GFP fluorescence of leaves were visualized with blue light-emitting-diode source (Biorad ChemiDoc MP).

FIG. 4. Quantitation of anthocyanins in field samples used for sRNAseq differential expression analysis. Asterisk (*) indicates significantly different than asymptomatic control samples, p<0.04 (Student's t test, equal variance assumed).

FIG. 5. CleaveLand [159] T plots of grapevine degradome evidences for A) miR7122 slicing of TAS-14s0081g00100 PHAS locus [148] to generate 3′D16(+) tasiRNA. B) RD22 BURP domain homologue of genes listed in Table IV being sliced by novel TAS-14s0081g00100 tasiRNA3′D16(+) derived from miR7122.

FIG. 6. Control (SRI, left) and heterozygous AtMYB90 tobacco plants (right) super-transformed with pCAM-V2 in rooting medium.

FIGS. 7A and 7B. pMAL protein expression FIG. 7A, pMAL-c5x-V2; FIG. 7B, pMAL-c5x-C2.

FIGS. 8A to 8C. pMAL-c5x-C2 protein expression at 18° C. for 18 hours FIG. 8A, T7 shuttle with 0.3 mM IPTG; FIG. 8B, NEB Express with 0.3 mM IPTG; FIG. 8C, NEB Express with 0.1 mM IPTG.

FIGS. 9A and 9B are restriction enzyme digestion FIG. 9A, pGBTK7-BD-C2; FIG. 9B, pGBTK7-BD-V2 *EcoRI digestion displayed star activity.

FIGS. 10A and 10B are restriction enzyme digestion FIG. 10A, pART27-hpC2; FIG. 10B, pART27-hpV2 *NotI digestion displayed star activity.

FIG. 11. Current status of somatic embryogenesis to regenerate transgenic plantlets from two rounds of Agrobacterium-mediated anther-derived callus transformation and selection.

FIGS. 12A and 12B shows the cloning of GRBV viral partial dimer constructs for agroinfection functional assays [10]. FIG. 12A is a schematic representation of partial dimer cloning. Triangles refer to tandem repeats of the common region (CR) that recombine in host to release an infectious viral genome nicked by viral replicase and amplified by rolling circle replication [10]. FIG. 12B is a restriction enzyme digestion of full-length viral clone in pBSII-KS⁺.

FIG. 13 a pHANNIBAL-C2 vector of the present invention with a C2 insert, PDK insert under the control of a p35S promoter, f1 ori, Amp resistance, bacterial ori and Ocs 3′.

FIG. 14 a pHANNIBAL-hpC2 vector of the present invention with two C2 inserts in opposite directions, PDK insert under the control of a p35S promoter, fl ori, Amp resistance, bacterial ori and Ocs 3′.

FIG. 15 a pHANNIBAL-V2 vector of the present invention with a V2 insert, PDK insert under the control of a p35S promoter, fl ori, Amp resistance, bacterial ori and Ocs 3′.

FIG. 16 a pHANNIBAL-hpV2 vector of the present invention with two V2 inserts in opposite directions, PDK insert under the control of a p35S promoter, fl ori, Amp resistance, bacterial ori and Ocs 3′.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Grapevine Red Blotch Virus (GRBV) is a monopartite, grapevine-infecting Grablovirus causing Red Blotch Disease and was first observed in California in 2008. GRBV is a serious threat to North American vineyards that the Pierces Disease/Glassy-Winged SharpShooter Board (PD/GWSSB) of the California Department of Food and Agriculture (CDFA) is addressing by investing in applied research focused on animal vectors, epidemiology, ecology, and field transmission. An understanding of the viral gene functions and molecular mechanisms evolved by GRBV to mount successful infection and disease states is essential to develop resistance strategies against the virus that threaten a multi-billion dollar industry. Consistent with geminiviruses, open reading frame (ORF) predictions confirm transcription of six GRBV genes is bidirectional but experimental elucidation of gene function is lacking. RNA silencing has evolved as a major host defense mechanism against the invasive pathogens. The presence of a robust viral counter defense machinery is underscored by the ubiquitous presence of one or more silencing suppressor proteins in plant viral genomes. The arms race between silencing and silencing suppression results in resistance or susceptibility to the pathogen. The inventors took a comprehensive approach by cloning all the viral ORFs from GRBV-infected vines to test for GRBV silencing suppressor proteins. The inventors identified two silencing suppressor proteins C2 and V2 encoded by GRBV. The inventors made recombinant DNA hairpin vectors targeting C2 and V2, which will be used to generate stably transformed transgenic grapevine plants which will be tested for GRBV resistance. To identify the host targets of the viral suppressor proteins the suppressor protein genes in expression vector pMAL-c5X and yeast two-hybrid bait recombinant vector pGBTK7-BD were cloned.

The anthocyanin levels in dicot leaves are under a tightly controlled regulatory mechanism involving endogenous small RNAs (sRNAs). The red patches in the interstitial lamina of GRBV-infected leaves and in petioles and veins are caused by deranged anthocyanin accumulation, a well-known stress response in plants. It is plausible that the apparent rapid spread of GRBV by arthropod vector(s) could be driven in part by visual or olfactory cues from symptomatic grapevines.

The relationship between wine grapes and virus diseases is similar to that between humans and health problems; they affect a wide range vital characteristics and have many modes of action. The etiology and epidemiology of Grapevine Red Blotch Virus (GRBV) remains unknown, but its discovery was originally delayed due to ‘confirmation bias’; it was thought to be a new strain of the major virus Grapevine Leaf Roll associated Virus (GLRaV) [1, 2]. GLRaV-3 is the most common and widespread, accounting for ˜60% of the global grape production losses due to virus diseases (http://wine.wsu.edu/extension/grapes-vineyards/grape-diseases/virus-diseases/). Red blotch disease management draws on understanding infection biology, ecological factors influencing spread, tritrophic virus-vector-host interactions, and improvements in diagnostic technologies [2]. The inventors found that understanding the viral gene functions and effects on host physiology and molecular mechanisms of genomic regulation are necessary to effectively combat Red Blotch disease. Understanding how GRBV causes disease can present cogent strategies for mitigating this threat to a multibillion-dollar industry. Degradation of viral transcripts (RNA silencing) has evolved as a major host defense mechanism against invasive pathogens. Viruses counter the plant defense mechanisms by evolving one or more “silencing suppressor” proteins. The efficacy of host silencing versus viral silencing suppression results in resistance/tolerance or susceptibility to the pathogen. The red color of leaves is caused by anthocyanin, a color pigment tightly controlled regulatory mechanism involving endogenous small RNAs (sRNAs). The red patches in the interstitial lamina of GRBV-infected leaves and in petioles and veins are caused by deranged anthocyanin accumulation, a well-known stress response in plants.

As used herein, the term “RNA interference” refers to a process in which a double-stranded RNA molecule changes the expression of a nucleic acid sequence with which the double-stranded or short hairpin RNA molecule shares substantial or total homology. While not being bound by theory, the mechanism of action may include, but is not limited to, direct or indirect down regulation of the expression of the C2 and/or V2 genes, decrease in C2 and/or V2 mRNA. The term “RNAi” includes an RNA sequence that elicits RNA interference, which can also be transcribed from a recombinant DNA vector. Also used herein, the terms “short hairpin RNA” or “shRNA” refer to an RNA structure having a duplex region and a loop region that may be used to target the C2 and/or V2 genes, in which the RNAis are expressed initially as shRNAs. Both shRNA and RNAi are encompassed by the present invention.

As used herein, the term “RNAi expression cassette” refers to a cassette having at least one promoter that drives the transcription of the RNAi, which can also be followed by a termination sequence or unit. In some instances, a recombinant DNA vector for use with the present invention may include multiple promoters upstream from the RNAi expression cassette. Thus, the terms “RNAi expression construct” or “RNAi expression vector” refer to vectors that include at least one RNAi expression cassette that targets the C2 and/or V2 genes.

Often, RNAi is optimized by using identical sequences between the target and the RNAi, however, RNA interference can be found with less than 100% homology. If there is less than 100% homology, e.g., 99%, 98%, 97%, 96%, or even 95%, 94%, 93%, 92%, 91% or even 90%, the complementary regions must be sufficiently homologous to each other to form the specific double stranded regions. The precise structural rules to achieve a double-stranded region effective to result in RNA interference have not been fully identified, but approximately 70% identity is generally sufficient. Accordingly, in some embodiments of the invention, the homology between the RNAi and C2 and/or V2 genes is at least 70%, 80%, 85%, 90%, or even 95% nucleotide sequence identity, so long as the expression of C2 and/or V2 genes is significantly lowered.

A common consideration for designing RNAi for targeting C2 and/or V2 genes, is the length of the nucleic acid or the insert of a recombinant DNA vector, for example, it is known that 17 out of 21 nucleotides is sufficient to initiate RNAi, but in other circumstances, identity of 19 or 20 nucleotides out of 21 may be required. While not being bound by theory, greater homology is commonly used in the central portion of a double stranded region than at its ends. The RNA expression products of the RNAi expression cassette lead to the generation of a double-stranded RNA (dsRNA) complex for inducing RNA interference and thus down-regulating or decreasing expression of the C2 and/or V2 genes.

The term “homology” refers to the extent to which two nucleic acids are complementary. There may be partial or complete homology. A partially complementary sequence is one that at least partially inhibits a completely complementary sequence from hybridizing to a target nucleic acid and is referred to using the functional term “substantially homologous.” The degree or extent of hybridization may be examined using a hybridization or other assay (such as a competitive Polymerase Chain Reaction [PCR] assay) and is meant, as will be known to those of skill in the art, to include specific interaction even at low stringency.

The inhibition of hybridization of the completely complementary sequence to the target sequence may also be examined using a hybridization assay involving a solid support (e.g., Southern or Northern blot, solution hybridization and the like) under conditions of low or high stringency. Low or high stringency conditions may be used to identify the binding of two sequences to one another while still being specific (i.e., selective). The absence of non-specific binding may be tested by the use of a second target that lacks even a partial degree of complementarity (e.g., less than about 30% identity). In the absence of non-specific binding, the probe will not hybridize to the second non-complementary target and the original interaction will be found to be selective.

The art knows that numerous equivalent conditions may be employed to achieve low stringency conditions. Factors that affect the level of stringency include: the length and nature (DNA, RNA, base composition) of the probe and nature of the target (DNA, RNA, base composition, present in solution or immobilized, etc.) and the concentration of the salts and other components (e.g., formamide, dextran sulfate, polyethylene glycol). Likewise, the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, inclusion of formamide, etc.).

The present invention uses standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; Fitchen, et al. (1993) Annu Rev. Microbiol. 47:739-764; Tolstoshev, et al. (1993) in Genomic Research in Molecular Medicine and Virology, Academic Press; and Ausubel et al. (1992) Current Protocols in Molecular Biology, Greene/Wiley, New York, N.Y. Abbreviations and nomenclature, where employed, are deemed standard in the field and commonly used in professional journals such as those cited herein, and all relevant portions are incorporated herein by reference in their entirety.

As used herein, the term “gene” refers to a functional protein, polypeptide or peptide-encoding unit. As will be understood by those in the art, this functional term includes both genomic sequences, complementary/copy DNA (cDNA) sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “vector” is used in reference to nucleic acid molecules that transfer DNA segment(s) from one cell to another. The vector may exist in a state independent of the host cell chromosome, or may be integrated into the host cell chromosome.

As used herein, the term “disease resistance” refers to the ability of plants to restrict the activities of a specified pest, in this case the Grapevine Red Blotch Virus (GRBV). As used herein, the term “disease tolerance” refers to the ability of plants to endure an infection with Grapevine Red Blotch Virus (GRBV).

By way of explanation, and in no way a limitation of the present invention, the inventors hypothesize the viral suppressor protein(s) of GRBV interfere with the anthocyanin regulatory pathways and result in uncontrolled anthocyanin accumulation in vegetative tissues, thus serving as a visual cue for feeding by the assumed arthropod vector capable of transmitting the viruses. Thus, identifying the GRBV viral suppressor proteins and host targets is necessary to develop disease resistance strategies involving engineering and/or breeding for virus resistance going forward.

The inventors completed molecular validation of host silencing suppression by GRBV proteins. The inventors completed sRNA library sequencing and analysis of 13 symptomatic samples and 16 asymptomatic samples collected from Cloverdale field, Temecula field and Jacksonville, OR and Santa Rosa, Calif. samples. The inventors standardized the induction condition of pMAL-c5X recombinant DNA vector with C2 and V2. Large-scale protein purification for binding assays can be performed, and can also make the grape cDNA library with GRBV infected grapevine for yeast-two-hybrid (Y2H) assay. Finally, a binary recombinant DNA vector cloning of hpRNA vector targeting the GRBV suppressor proteins was completed and electroporated into Agrobacterium tumefaciens EHA105, and shipped the strains to Cooperator Tricoli for transformation and regeneration of rootstock 101-14. Started cloning and sequencing of virus for testing the transgenic plants for disease resistance is conducted.

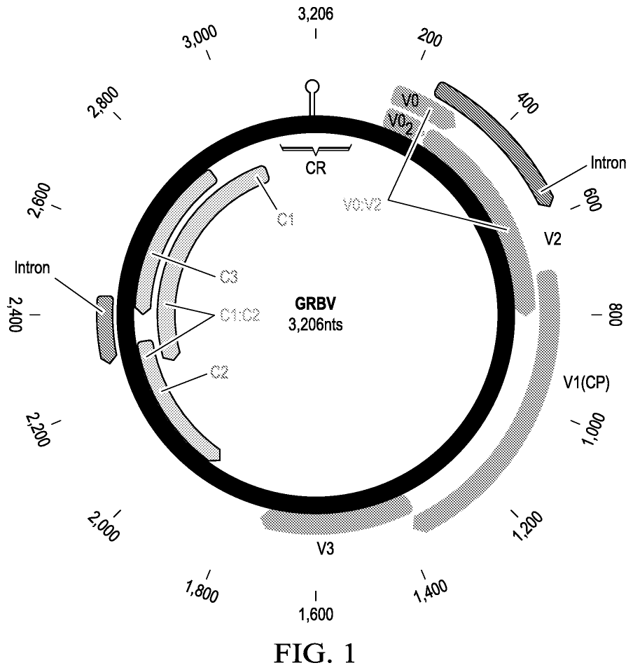

To date, 120 full-length GRBV genome sequences have been deposited in NCBI GenBank corresponding to two phylogenetic clades with limited 4-9% intra-clade divergence; no geographic or cultivar specificity, nor biological difference between the two types of variants is known [2]. Consistent with geminiviruses, GRBV possesses the conserved nonanucleotide sequence and seven putative overlapping open reading frames (ORF) are transcribed bidirectionally [25]. GRBV encodes up to five ORFs in the virion strand (V0, V02, V1, V2, V0: V2 spliced fusion, and V3) [33] and three in the complementary strand (C1, C2, C1:C2 spliced fusion, and C3; FIG. 1). Similar to mastrevirus (a monopartite geminivirus), GRBV complementary-sense ORF C1 encodes RepA, Replication-associated protein. Another spliced transcript encompassing the C1 and C2 ORFs encodes Rep, the Replication protein [12, 25, 34, 35]. GRBV virion-sense strand ORFs V2 and V3 are predicted to encode movement proteins, whereas V1 ORF encodes coat protein. V2 protein localizes in the nucleoplasm, Cajal bodies, and cytoplasm; the V3 protein localizes in various unidentified subnuclear bodies. Additionally, the V2 protein is redirected to the nucleolus upon co-expression with the nucleolus and Cajal body-associated protein Fib2 [36]. FIG. 1. Genome Organization of Grapevine red blotch virus (GRBV), previously called Grapevine red blotch-associated virus (GRBaV). The core region (CR) of ˜200 nucleotides contains bidirectional tissue-specific promoters and an inverted repeat that forms a hairpin loop with an invariant 9-nt 5′-TAATATT↓AC-3′ that is nicked by the viral replicase and acts as the origin of Virion (V) strand DNA replication in host. Modified from [33]

The functions of the predicted GRBV ORFs are yet to be elucidated experimentally. Understanding the molecular mechanisms by which the virus mounts a successful infection is fundamental and essential to develop cogent engineered resistance strategies. A practical issue is that the few proteins encoded by geminiviruses are multifunctional and likely modulate several host regulatory genes, a mechanism uniquely evolved by the viruses to balance the genome size-constraint emplaced by the capsid. A comprehensive ‘omics’ profiling experiment on berry development and select metabolite and enzyme quantitations in GRBV-infected grapes from two different vineyards suggested several host regulatory pathways, in particular phenylpropanoids, are impacted by the virus [37]. GRBV infection results in deranged expression of host post-transcriptional machinery, transcription factors, and several hormone biosynthesis and response pathways. Post-transcriptional gene silencing (PTGS) processes involving microRNAs (miRNAs) and small interfering RNAs (siRNAs) are known to regulate host immune responses

TABLE I
Suppressor proteins characterized in Geminivirus and their plant targets.

| Virus* | Suppressor | Suppressing PTGS | Suppressing TGS | Cellular pathways |
|---|---|---|---|---|
| MYMV | AC2 | Upregulate host | | |
| | | suppressor protein | | |
| | | WEL1[38] | | |
| TGMV | AL2 | Inactivate Adenosine | | Inactivate a serine-threonine |
| BCTV | L2 | kinase [39, 40] | | kinase SnRKI [42] |
| | | Upregulate rgs-CaM [41] | | |
| BSCTV | C2 | Stabilize S-adenosyl | | |
| | | methionine | | |
| | | decarboxylase l | | |
| | | (SAMDCI) [43] | | |
| TGMV | AL2 | | Inactivate Adenosine kinase; stabilize SAMDCI [44] | |
| CaLCuV | AL2 | | Inhibit histone Me-transferase SUVH4/KYP [45, 46] | |
| BCTV | L2 | | | |
| TGMV | AL2 | | | Elevation of cellular cytokinin |
| SCTV | C2 | | | levels [47] |
| TYLCSV | C2 | | | Interact with CSN5 and inhibit |
| | | | | jasmonate signaling [48, 49] |
| ICMV -SG | AC2 | | Upregulation of | |
| | | | RAV2, transcription | |
| | | | repressor [50] | |
| MYMIV | AC2 | Interact with RDR6 and | | |
| | | AGO1 [51] | | |
| TYLCV | C2 | | | Downregulate terpene synthesis |
| | | | | [52] Binding to ubiquitin [53] |
| ToLCTWV | C2 | | | Suppress CMT3 expression [54] |
| ACMV | AC4 | Binds ss miRNA [55] | | |
| WDV | Rep | Binds ss-and duplexed 21 | | |
| | | and 24 nt siRNAs [56] | | |

| | | |
|---|---|---|
| TYLCV | V2 | Compete NbMETl for binding to histone deacetylase6 |
| | | [57]; SGS3 [58]; AGO4 [59] |
| CLCuMuV | V2 | AGO4 [60] |
| GGVA | V2 | Mechanisms not known [61, 62] |
| MMDaV | | |

*Acronyms: Mungbean yellow mosaic virus: MYMV; Tomato golden mosaic virus: TGMV; Beet curly top virus: BCTV; Beet severe curly top virus: BSCTV; Cabbage leaf curl virus: CaLCuV; Spinach curly top virus: SCTV; Tomato yellow leaf curl Sardina virus: TYLCSV; Tomato yellow leaf curl virus: TYLCV; African cassava mosaic virus: ACMV; Wheat dwarf virus: WDV Mungbean yellow mosaic India virus: MYMIV; Indian cassava mosaic virus-Singapore: ICMV -SG; GGVA-Grapevine geminivirus A; MMDaVMulberry mosaic dwarf-associated virus;

to viruses and microbes, as well as normal plant development and hormonal signaling [63, 64].

PTGS has evolved as a major host defense mechanism against invasive pathogens including viruses. The presence of a robust viral counter defense mechanism is underscored by the ubiquitous presence of one or more silencing suppressor proteins in the genomes of many plant viruses. The “arms race” between host silencing of pathogen transcripts and silencing suppression by pathogen gene products results in resistance or susceptibility to the pathogen. Numerous geminiviruses encode silencing suppressor proteins that target PTGS, transcriptional gene silencing (TGS), and cellular regulatory genes (Table I, above). The layers of complexity employed by geminiviruses to target multiple host antiviral processes pose significant challenges to devise engineered strategies for crop viral resistance.

Notwithstanding the complexity of geminivirus-host interactions, transgenic approaches involving over-expression of viral coat protein has been very successful in developing commercially produced Papaya ring spot virus (PRSV)-resistant papaya [65, 66], Potato virus X and Y resistant potato [67], and Squash mosaic virus resistant squash [68]. The only successful report of engineering geminiviral resistance using coat protein was in tomato against TYLCV [69]. Transgenes of RepA protein have been successfully deployed to generate geminivirus resistance [70-72]. Mutants of many geminiviral genes have been evaluated for trans-dominant negative inhibition of geminivirus replication and movement [73-76]. The major limitation of introducing geminivirus sequences into transgenic plants was that in several cases the transgenic protein facilitated viral replication [77, 78]. The expression of gene5 protein (g5p) from E. coli phage M13 [79] and transgenic expression of Agrobacterium VirE2 [80-82], a ssDNA binding protein essential for virulence, are resistance strategies deployed against a broad spectrum of DNA viruses. Thus, the strategy of expressing non-viral proteins overcomes the limitations of functional/mutant viral proteins by not contributing to viral replication and can confer broad spectrum resistance to other geminiviruses.

Early reports of engineered geminivirus resistance, which serendipitously involved host RNA silencing before its significance was understood, were by expressing sense and antisense viral RNAs in plants. Expression of AC1 in antisense orientation conferred resistance against TGMV, BGMV and TYLCV [83-86], whereas expression of various Cotton leaf curl virus genes in antisense orientations in tobacco conferred resistance [87]. Transient expression of the hpRNA gene of the MYMV bidirectional promoter [88], ACMV-[CM] Rep siRNA [89] and MSV Rep hpRNA gene [90] conferred resistance against the respective viruses. The hpAC1/C1 genes conferred resistance against TYLCV in tobacco [91], BGMV in common bean [92, 93] and ACMV in cassava [94]. Transgenic expression of hpRNA from the bidirectional promoter of ACMV in cassava [95] and TYLCV CP promoter in tomato [96] conferred resistance against the respective viruses. Silencing the viral suppressor protein by transgenic expression of hpAC1 and hpAC4 of ToLCV in tomato [97], hpAC4 [98] and hpAC2 of MYMV [99] have proven to be a very effective strategy in conferring resistance.

Previous work on the model plant Arabidopsis in the inventors' lab showed altered source-sink distributions of sucrose and the stress hormone abscisic acid (ABA) [100] interact to regulate anthocyanin accumulation via miR828, Trans Acting Small-interfering locus4 (TAS4), and their target MYeloBlastosis viral oncogene-like (v-MYB) transcription factors, viz. Vvi-MYBA6/7 and close homologues targeted by miR828 in grapevine [101, 102]. GRBV infections result in higher quantities of carbohydrates in symptomatic leaves [29], suggesting deranged sugar signaling may play a role in the expression of red leaf symptoms. The inventors characterized the conserved autoregulatory loop involving miR828 and TAS4 down-regulates anthocyanin biosynthesis by targeting MYB transcription factors induced by UV light in grape [103]. The recently published transcriptome profiling study of GRBV-infected host berries identified significant repression of rate-limiting ABA biosynthesis loci NCED2/3 (first described by the inventors [104]) in infected berries [37].

A working model is that GRBV infection interferes with the normal PTGS pathways of the host by the activity of viral-encoded suppressor proteins. The possibility exists that mixed infections of GLRaV, GRBV or other grapevine viruses like Pinot gris virus [2] and latent grapevine fleck virus (GFkV) [105] result in interactions in arthropod vectors or host causing synergistic effects and more severe damage/symptoms [106-108]. It is also possible that apparent rapid spread could be driven by vector visual or olfactory cues taken from infected vines that translate to insect vector feeding preferences. microRNAs, trans-acting small-interfering, and phased small RNAs (miRNAs/tasi-RNAs/phasi-RNAs) regulate a large array of host gene expression at the post-transcriptional level and transcriptional levels [109]. Viruses target plant miRNAs to facilitate pathogenesis, and plants have co-opted miRNAs for innate immunity [110-113]. Their collective changes in virus-infected and engineered transgenic tissues that results in susceptibility [114, 115] supports their functions as master regulators targeted by pathogens. Broader roles for plant sRNAs in evolutionary adaptations [116, 117] may include virus arthropod vector feeding processes and olfactory preferences. By way of explanation, but in no way a limitation of the present invention, the inventors hypothesized the red blotch phenomena observed in GRBV-infected grape leaves is a consequence of viral suppressor proteins targeting the miR828/TAS4/MYBA5/6/7 autoregulatory loop [100, 103] which fine tunes anthocyanin levels by a “rheostat” feedback [103].

TABLE II
Analysis of publicly available transcriptome data\hat{ } for GRBV-infected berries across
development.

| developmental stage: | | | pre-veraison | | versison | | Post-veraison | | harvest | |
|---|---|---|---|---|---|---|---|---|---|---|
| | | Phase | beta~ | | beta~ | | beta~ | | beta~ | |
| target; sRNA effector | gene ID | Score | LFC | pval | LFC | pval | LFC | pval | LFC | pval |
| GRBa V genome | JQ901105.2 | n.d. | 6.26 | 1.91E−15 | NA | NA | NA | NA | 6.76 | 3.47E−32 |
| Vvi-TAS4c; miR828 | chr1:2961251:2961747 | 3375 | NA | NA | −1.01 | 0.13 | −1.01 | 0.13 | 0.38 | 0.53 |
| AGO1a; miR168/530 | VIT_17s0053g00680 | n.d. | 0.06 | 0.55 | 0.17 | 0.04 | 0.17 | 0.04 | 0.16 | 0.05 |
| AGO1b; miR168/530 | VIT_19s0014g01840 | n.d. | 0.26 | 0.47 | 0.43 | 0.04 | 0.43 | 0.04 | 0.08 | 0.75 |
| MYBA6, TAS4 | VIT_14s0006g01290 | 22.2 | NA | NA | NA | NA | NA | NA | 1.25 | 0.09 |
| MYB^{PALI}; miR828 | VIT_00s0341g00050 | 476 | 0.52 | 0.01 | 0.12 | 0.39 | 0.12 | 0.39 | 0.13 | 0.31 |
| MYB; miR828 | VIT_17s0000g08480 | 1330 | 0.62 | 0.09 | 0.33 | 0.35 | 0.33 | 0.35 | NA | NA |
| MYB; miR828 | VIT_04s0079g00410 | 24.6 | 0.39 | 0.01 | 0.17 | 0.04 | 0.17 | 0.04 | −0.06 | 0.46 |
| AGO2a; miR403 | VIT_10s0042g01180 | 50 | 0.61 | 0.02 | 0.36 | 0.07 | 0.36 | 0.07 | 0.82 | 0.02 |
| AGO2b; miR403 | VIT_10s0042g01200 | n.d. | 0.04 | 0.81 | 0.03 | 0.81 | 0.03 | 0.81 | −0.16 | 0.29 |
| DCL2; unknown | VIT_04s0023g00920 | 33.8 | 0.39 | 0.25 | 0.47 | 0.03 | 0.47 | 0.03 | 0.11 | 0.57 |
| SGS3; unknown | VIT_07s0130g00190 | 177.4 | 0.04 | 0.69 | 0.23 | 0.01 | 0.23 | 0.01 | 0.16 | 0.06 |
| DCL1; miR162 | VIT_15s0048g02380 | n.d. | −0.05 | 0.62 | 0.05 | 0.54 | 0.05 | 0.54 | −0.21 | 0.15 |

\hat{ } Oakville vineyard dataset (ref.³⁷) analysed by kallisto/sleuth.

A recent paper reported GRBV effects on berry development [37]. Table II provides evidence drawn from publicly available berry transcriptome data which supports this model. A large (˜Log2 fold-change˜−1.46; beta=−1.01) downregulation of Vvi-TAS4c at veraison and post-veraison in GRBV-infected berries is seen, albeit not statistically significant, suggesting the miR828-TAS4-MYB pathway could be a specific target of GRBV. This is supported by the strong up-regulation of MYBA6 at harvest, the target of a deeply conserved TAS4c tasi-RNA 3′D4(−) along with several other MYBs [102, 103] shown to function in the phenylpropanoid/flavonol pathway and targeted by miR828.

Interestingly, a significant up-regulation of ARGONAUTE (AGO), DICER2, and SUPPRESSOR_OF_GENE_SILENCING3 (SGS3) transcripts was observed, all major effectors of the PTGS machinery required for viral resistance [118, 119], and themselves subject to PTGS and spawning of amplified phasi-RNAs [120-122]. It will be very interesting to determine if transitivity of these loci and MYBA5/6/7 is deranged by GRBV. One reason is because the “21₁ mechanism” of transitivity [123] in play with TAS4-3′D4(−) and target MYBA5/6/7 is novel and its significance is not understood, unlike the known ‘212’ and ‘221 hit’ mechanisms [123]. By way of explanation and in no way a limitation of the present invention, the inventors hypothesized repression of silencing machinery upon virus infection, but the evidence is that the host is compensating by overexpressing PTGS effector pathways. These results underscore the need to perform transcriptome and sRNA analyses from different tissues of field-infected grapevines.

The present inventors were able to: Validate the identified candidate GRBV suppressor proteins C2 and V2. Elucidate by a system's approach the molecular mechanisms by which GRBV causes symptoms from genome-wide analyses of host microRNAs (miRNAs), trans-acting small interfering (tasi-) RNAs, phasi-RNAs, and effects on host target mRNAs by RNA-Seq and degradome analyses of (a) field samples, and (b) of tobacco genotypes over-expressing GRBV C2 and V2 suppressor proteins and an effector of anthocyanin, AtMYB90/PRODUCTION_OF_ANTHOCYANIN2(PAP2). Identify the host grapevine targets of GRBV suppressor proteins C2 and V2 Design transgenic grapevine experiments to test disease resistance of transgenic grape expressing hairpin silencers directed to GRBV suppressor protein transcripts.

Example 1. Validate the Identified Candidate GRBV Suppressor Proteins C2 and V2.

The inventors characterize GRBV suppressor proteins. The inventors cloned GRBV genes V1, V3, C1 and C3 with HindIII/SacI flanking sites, and V2/C2 with HindIII/EcoRI sites from genomic DNAs of GRBV-infected grape leaf tissue collected in 2016 from ‘Calle Contento’ vineyard (cv. Merlot) in Temecula Calif. into the corresponding sites of pJIC-35S recombinant DNA vector [124]. The pJIC-35S-ORF cassettes was subsequently cloned into the binary vector pCAMBIA2301 and electroporated into A. tumefaciens strain EHA105. To evaluate if GRBV possesses viral silencing suppressor proteins, N. benthamiana line 16c, developed in the laboratory of Sir David Baulcombe [125] expressing A. victoria jellyfish Green Fluorescence Protein (GFP) was used as the test system. In this system, RNA silencing of the gfp transgene can be triggered by transient expression of a gfp (trigger)-expressing recombinant DNA vector. Consequently, the agroinfiltrated leaf will exhibit loss of GFP and manifest red auto-fluorescence from chlorophyll. When a silencing suppressor protein gene is co-infiltrated along with gfp (trigger), the infiltrated zone will exhibit rescue of green fluorescence as marker of suppression of GFP RNAi silencing.

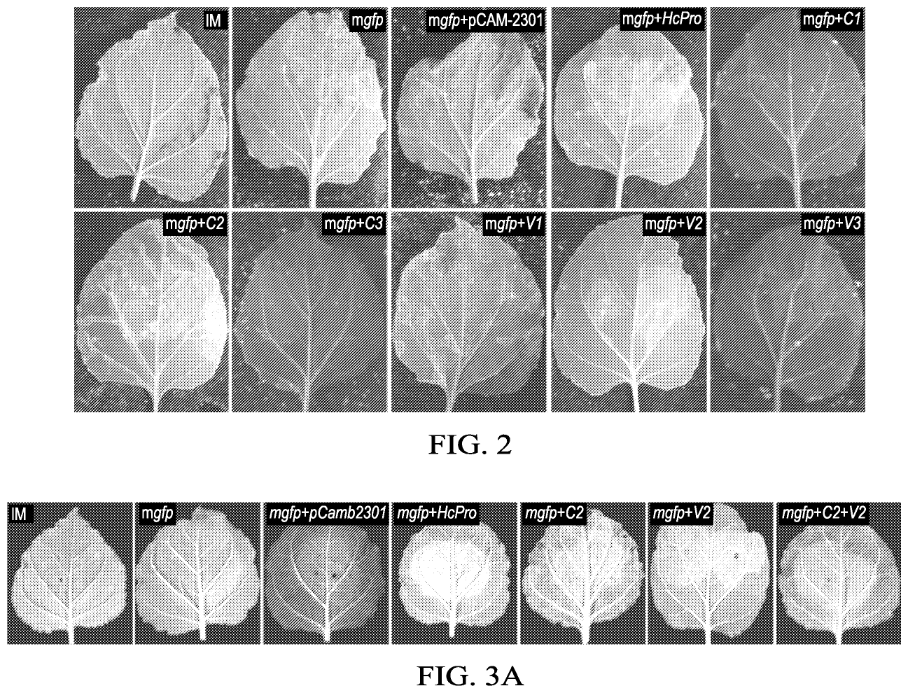

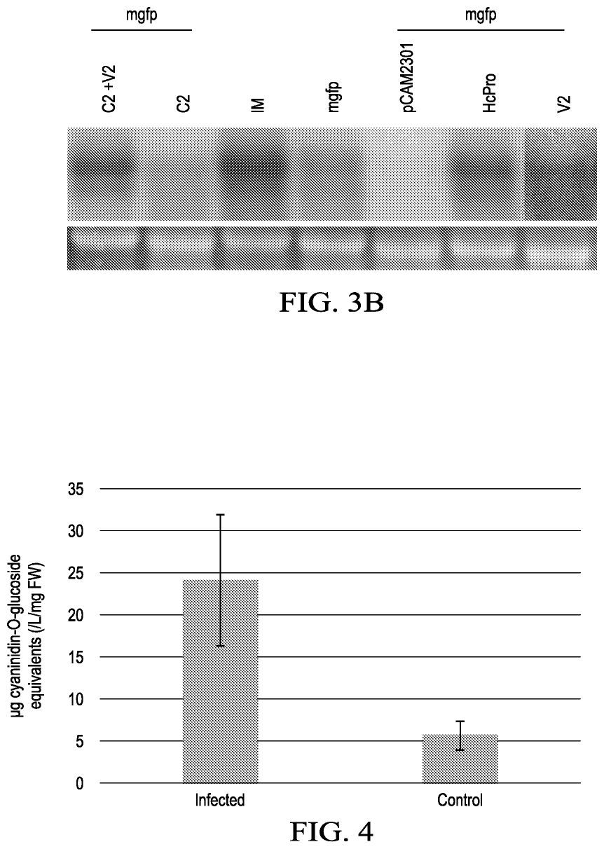

Six-week-old N. benthamiana 16c plants were agroinfiltrated with the A. tumefaciens strain harboring the p35S-gfp (pBI-mgfp5-ER; the ‘trigger’) and p35S-gfp+pCAMBIA-2301 with or without co-infiltration of test GRBV constructs. Potyvirus HcPro [126] construct co-infiltration served as positive control for silencing suppression. Five days post infiltration, local GFP silencing of infiltrated leaves was observed under long wave UV light as red auto-fluorescence (FIG. 2: mgfp; mgfp+pCAM−2301). To evaluate the silencing suppression effect of GRBV gene products, the 16c plants were agroinfiltrated with 1:1 test mixture of the A. tumefaciens strains harboring p35S-gfp(trigger) with p35S-V1/p35S-V2/p35S-V3/p35S-C1/p35 S-C2 or p35 S-C3, respectively. As expected, bright green fluorescence was observed in the infiltrated zones with mgfp+HcPro co-inoculation (FIG. 2 mgfp+HcPro). GRBV C1, C3, V1 and V3 construct co-infiltration did not suppress the silencing triggered by mgfp (FIG. 2: mgfp+C1; mgfp+C3; mgfp+V1; mgfp+V3). In the presence of GRBV C2 and V2 expressions from co-infiltrated constructs, the infiltrated area was not silenced by the gfp(trigger) (infiltration zones display green fluorescence; FIG. 2: mgfp+C2; mgfp+V2). Thus, GRBV C2 and V2 proteins are identified as candidate suppressor proteins. This result has been repeated, providing compelling evidence for C2 and V2 functions as GRBV silencing suppressors (FIGS. 3A and 3B). The inventors also found evidence for additive effect of C2+V2 in suppressing silencing by co-expressing the two genes (FIGS. 3A and 3B). FIG. 2. Agroinfiltration of the Nicotiana benthamiana 16c-GFP to demonstrate silencing suppression by HcPro and test GRBV genes. Agrobacterium-mediated transient expression in N. benthamiana leaves. IM: Mock-agroinfiltrated with infiltration medium. mgfp: Agroinfiltrated with the A. tumefaciens strain harboring p35S-gfp as control for GFP transgene silencing. mgfp+pCAM-2301: Co-agroinfiltrated with strains harboring p35S-gfp+pCAMBIA-2301 (negative control). mgfp+HcPro: Co-agroinfiltrated with strains harboring P35S-gfp+Potyvirus HcPro (Positive control for PTGS). mgfp+C/: Co-agroinfiltrated with strains harboring P35S-gfp+GRBV C1. mgfp+C2: Co-agroinfiltrated with strains harboring P35S-gfp+GRBV C2; Note PTGS suppression evidence, like HcPro. mgfp+C3: Co-agroinfiltrated with strains harboring P35S-gfp+GRBV C3. mgfp+VI: Co-agroinfiltrated with strains harboring P35S-gfp+GRBV VI. mgfp+V2: Co-agroinfiltrated with strains harboring P35S-gfp+GRBV V2. Note PTGS suppression evidence, like HcPro. mgfp+V3: Co-agroinfiltrated with strains harboring P35S-gfp+GRBV V3. Red chlorophyll autoflluorescence and green GFP fluorescence of leaves were visualized with hand-held UV-B light source.

Northern blot analysis of agroinfiltrated leaf tissue was performed using gfp gene. The agroinfiltrated area was harvested under UV light and total RNA was extracted and leaf sections were pooled from 5-8 technical replicates. Samples from mock infiltrated sections accumulated gfp transcript (FIG. 3B). Agroinfiltration of P35S-gfp-triggered silencing resulted in reduction of gfp transcript when infiltrated alone (mgfp) and total absence of gfp transcript when co-infiltrated with the empty binary vector pCAMBIA2301 (pCAMBIA2301+mgfp). Co-infiltration with mgfp+HcPro resulted in green fluorescence and accumulation of gfp transcript as expected. Co-infiltration with mgfp+GRBV V2 and mgfp+GRBV C2 which resulted in green fluorescence also resulted in accumulation of gfp transcript, albeit to a lesser level when compared to HcPro. Co-infiltration with mgfp+GRBV C2+V2 resulted in intense green fluorescence indicating an additive effect of two suppressor proteins (FIG. 3A) which was substantiated by the increased gfp transcript accumulation (FIG. 3B). Although the inventors observed conspicuous green fluorescence upon infiltration with mgfp+GRBV C2 and mgfp+GRBV V2 (FIGS. 2 and 3A), the northern results indicate the co-expression of C2+V2 together enhance the stability of mgfp transcript (FIG. 3B). FIGS. 3A and 3B show the validation of silencing suppression by GRBV genes C2 and V2. (FIG. 3A) Agrobacterium-mediated transient expression in N. benthamiana leaves. IM: Mock-agroinfiltrated with infiltration medium. mgfp: Agroinfiltrated with the A. tumefaciens strain harboring p35S-gfp as control for GFP transgene silencing. mgfp+pCAM-2301: Co-agroinfiltrated with strains harboring p35S-gfp+pCAMBIA-2301 (negative control). mgfp+HcPro: Co-agroinfiltrated with strains harboring P35S-gfp+Potyvirus HcPro (Positive control for PTGS). mgfp+C2: Co-agroinfiltrated with strains harboring P35S-gfp+GRBV C2; mgfp+V2: Co-agroinfiltrated with strains harboring P35S-gfp+GRBV V2; mgfp+C2+V2: Co-agroinfiltrated with strains harboring P35S-gfp+GRBV C2+GRBV V2 (FIG. 3B) Northern blot analysis with gfp probe. Total RNA from the infiltrated areas of mock infiltrated leaves (IM), P35S-gfp infiltrated leaves (mgfp), P35Sgfp+C2 (mgfp+C2) P35Sgfp+V2 (mgfp+V2) and P35Sgfp+HcPro (mgfp+HcPro), P35Sgfp+C2+V2 (mgfp+C2+V2) was probed with the gfp coding sequence. The 18S rRNA portion of the ethidium bromide-stained gel is placed at the bottom as control for RNA loadings. Red chlorophyll autoflluorescence and green GFP fluorescence of leaves were visualized with blue light-emitting-diode source (Biorad ChemiDoc MP).

GRBV C2 and V2 proteins were identified as candidate suppressor proteins, and methods (T-DNA binary effector constructs pCAM-C 1-gus and pCAM-V2-gus) and evidence presented in the Final Report for CDFA 18-0296-000-SA. V2 of GRBV is not homologous to any known geminivirus V2 proteins. Taken together the transient assay and gfp RNA blot provide conclusive evidence that GRBV genes C2 and V2 are viral suppressor proteins. The inventors also identified an additive effect of two suppressor proteins when co-expressed.

Example 2. Elucidate by a systems approach the molecular mechanisms by which GRBV causes symptoms from genome-wide analyses of host microRNAs (miRNAs), trans-acting small interfering (tasi-) RNAs, phasi-RNAs, and effects on host target mRNAs by RNA-Seq and degradome analyses of (a) field samples, and (b) of tobacco genotypes over-expressing GRBV C2 and V2 suppressor proteins and an effector of anthocyanin, AtMYB90/PRODUCTION OF ANTHOCYANIN2(PAP2).

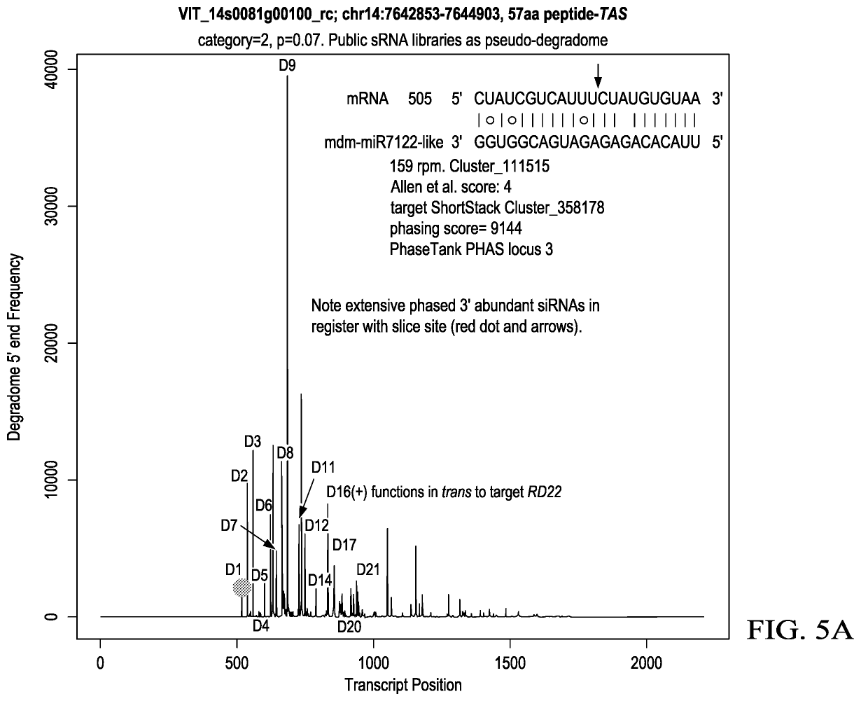

Unconfounded field samples that were GRBV-free were obtained to serve as negative controls for differential expression analysis of sRNAs and mRNAs by RNAseq. Table III summarizes the findings to date as they relate to 73 assayed field samples, where the inventors discovered mixed infections of GRBV with GLRaV strains 2 (NC_007448.1) and −3 (NC_004667.1) in Santa Rosa and Temecula vineyard samples collected based on visual screening for presence or absence of red blotch symptoms. Therefore, sRNA datasets for presence of reads were used for other emerging threat viruses Grapevine pinot gris (NC_015782.2), fanleaf (NC_003615.1, NC_003623.1, NC_003203.1), Grapevine viruses A and B (NC_003604.2, NC_003602.1), and fleck virus (NC_003347.1) as well as latent viruses (MF185002.1, KF137564.1, KF137565.1, KC427107.1,) and Xylella fastidiosa (AE009442.1), causal agent of Pierces disease. The inventors were able to identify 13 test libraries and 16 control libraries from different locations, years, and cultivars that were collected based on clinical field symptoms of presence or absence of red leaf blotches (FIG. 4). These 29 libraries were the basis of differential expression analysis of sRNAs by ShortStack [127] not confounded by known effectors of anthocyanin disease symptoms, specifically fanleaf, GLRaV, or Xylella. Fleck virus was identified in most of the Jacksonville OR samples, but no correlation between the GRBV sRNA differentially expressed (DE) results and presence of fleck virus (Table III; data not shown) was observed.

BSCTV C2 protein physically interacts with and stimulates host activity of S-adenosyl methionine (SAM) decarboxylase (SAMDC) to suppress SAM-mediated de novo methylation of viral DNA in Arabidopsis [43]. A viral-derived siRNA (vsiRNA) was elucidated as the pathogenicity determinant in TYLCV-infected tomato where it targets by near-perfect complementarity a host long non-coding RNA involved in development [128]. Recently a long-sought functional connection between RNA-dependent DNA Methylation (RdRM) and antiviral defense was established by the finding that the subnuclear Cajal body is the site of methylation of TYLCV DNA by physical interaction of host AGO4 with virus V2 protein, which blocks binding of AGO4 to viral RNA and DNA [59]. Additional evidence for vsiRNAs as pathogenicity determinants is that RNA virus Cucumber Mosaic Virus satellite Y produces a 22nt vsiRNA targeting protoporphyrin Mg-chelatase in tobacco to impair chlorophyll biosynthesis [129, 130]. This data-driven approach to discovery of GRBV pathogenicity and/or symptom determinants by quantifying host sRNAs and mRNAs by deep sequencing is concordant and complementary findings in the literature for host-pathogen interactions as described below.

TABLE III
Evidence of GRBV spread and/or mixed GLRaV infections based on sRNA-seq of
(a)symptomatic field samples, or from vines previously assayed PCR-negative for GRBV

| Location, year | Host cultivar | Number of | | percent non- |
|---|---|---|---|---|
| | | assayed vines | Number of | GRBV viral |
| | | (assumed | viral | infections; |
| | | GRBV- | sRNA- | GRBV inferred |
| | | negative, per | positive | spread percent |
| | | asymptomatic) | samples | over time, bold |
| Temecula CA 2018 | Merlot | 2^{Δ} | 1* | 50 |
| Temecula CA 2019 | Merlot | 4^{Δ} | 4* | 100 |
| Cloverdale CA 2018 | Cabernet franc | (10) | 2 | 20 |
| Santa Rosa CA 2019 | Pinot noir Pomard4 | (8) | 3^{†} | 38 |
| Santa Rosa CA 2019 | Pinot noir Pomard4 | 9^{Δ¶} | 6^{†} | 67 |
| Jacksonville OR 2018 | Pinot noir (Deboer; | 3^{§} | 2 | 67 |
| | parcel rogued 2019) | | | |
| Jacksonville OR 2019 | Pinot noir 777 | 13^{§£} | 5 | 38 |

^{Δ}Symptomatic anthocyanin accumulation in leaves
*GLRaV3 positive
^{†}GLRaV2 positive
^{¶}three samples tested GRBV positive
^{§}vine petioles PCR assayed GRBV negative the year before
^{£}Twelve of the 13 samples tested positive for grapevine fleck virus; only five for GRBV

FIG. 4. Quantitation of anthocyanins in field samples used for sRNAseq differential expression analysis. Asterisk (*) indicates significantly different than asymptomatic control samples, p<0.04 (Student's t test, equal variance assumed).

TABLE IV
Correlations of significant miRNA differential expressions from sRNA-seq of
symptomatic field samples, with observed differential expression of validated targets [103] (novel
validated in bold) in asymptomatic 2018 Jacksonville field samples.

| | | | | Target | Target | | |
|---|---|---|---|---|---|---|---|
| miRNA/TAS | RPM | L2FC | pval | MeanX | L2FC | pval | Target locus |
| GRBV siRNAs | 5,062 | 10.21 | 10⁻⁵⁷ | 44.9 k | 10.43 | 10⁻⁹ | See section below |
| miR2950 | 6.6 | 2.94 | 10⁻⁷ | 13 | −2.06 | 0.23 | Chlorophyllases† |
| | | | | | | | 07s0151g00110 |
| | | | | | | | 07s0151g00250 |
| | | | | | | | 07s0151g00190 [131, |
| | | | | | | | 132]; Fboxest† |
| | | | | | | | 18s0072g00820 |
| | | | | | | | 19s0014g01860 |
| miR3632/482-L | 10.3 | 1.36 | 0.001 | 366 | −0.47 | 0.45 | PRF Disease Resist |
| | | | | 104 | 2.72^{¶} | .0002 | RPS4/TAS5-like |
| miR398c | 10.1 | 2.48 | 0.001 | 568 | −0.15^{¶} | 0.78 | Cu/Zn chaperone |
| miR399i | 4.0 | 2.48 | 0.002 | n.d. | | | PHO84/PHT1_3 |
| RNASeq: | 0.5 | 1.31 | 0.82 | | | | |
| miR395g\hat{ } | 3.1 | −6.22 | 0.002 | 21 | −2.16 | 0.07 | SULTR2 |
| miR3627 | 27.4 | 1.87 | 0.004 | 832 | 0.13^{¶} | 0.79 | ACA10 Ca²⁺ transprt |
| miR3624a,b | 2.6 | 2.80 | 0.004 | 15 | −0.70 | 0.56 | Metal bind Pro-rich |
| RNAseq: | 7.8 | 2.42 | 0.24 | | | | |
| miR169g* | 1.4 | −2.34 | 0.008 | 129 | 0.88 | 0.16 | NFYA1_2 |
| miR169x | 0.6 | 2.81 | 0.01 | 3423 | −0.41 | 0.38 | JAZ repressor |
| miR408 | 26.6 | 1.48 | 0.01 | 51 | 2.88^{¶} | 0.004 | PLASTOCYANIN |
| miR166c | 39.3 | 3.65 | 0.01 | 332 | 0.94^{¶} | 0.09 | Homeobox HB8_3 |
| RNAseq: | 14 | 0.66 | 0.63 | | | | |
| miR168 | 67.4 | 1.24 | 0.01 | 2074 | 0.22^{¶} | 0.66 | ARGONAUTE1 |
| RNAseq: | 21 | 0.24 | 0.82 | | | | |
| miR530b-isomiR | 0.2 | 2.70 | 0.02 | 212 | −0.28 | 0.61 | ARGONAUTE1-like |
| miR3634 | 13.9k | 1.06 | 0.02 | 27 | −0.35 | 0.74 | FGGYcarbokinase |
| RNAseq: | | | | | | | 19s0085g00430 |
| | | | | 5 | 1.14^{¶} | 0.58 | Zinc Finger |
| | | | | | | | 18s0001g02000 |
| | 8 | −0.79 | 0.61 | | | | |
| novel | 1.2 | 1.52 | 0.02 | 2126 | −0.03 | 0.95 | NRT1.6-like |
| miR13/12/14 | | | | | | | 16s0050g01990 |
| novel miR7^{†} (24) | 7.5 | 1.07 | 0.03 | | | | n.a. |
| miR156b* | 3.2 | 1.68 | 0.03 | 137 | −0.92 | 0.14 | SPL6/LIGULELESS |
| miR3435/10978- | 1.1 | 0.96 | 0.04 | | | | n.a. |
| like^{†}, novel | | | | | | | |
| miR403a | 4.5 | −2.74 | 0.07 | 32 | 0.63 | 0.52 | AGO2-like, PHAS |
| RNAseq: | 0.6 | −3.77 | 0.49 | | | | |
| miR7122, TAS | 2.4 | 1.49 | 0.11 | | | | RD22f,h,m,d PHAS |
| 14s0081g00100 | | | | 119 | −4.59 | 10⁻⁷ | 04s0008g04020t |
| D16(+) RD22 | | | | 99 | −3.61 | 10⁻⁵ | 04s0008g04050t |
| trigger, TAS | 46.5 | 0.50 | 0.28 | 1347 | −1.87 | 10⁻⁴ | 04s0008g04150t |
| 3’D9(−) marker | | | | 57 | −1.64 | 0.07 | 04s0008g04000t |
| | | | | 2 | −4.99 | 0.23 | 04s0008g04120t |
| Known/hypothesized targets/effectors of viral silencing suppressors | | | | | | | Function/VIT_gene |
| miR828 RNASeq | | | | 6 | −0.06 | 0.97 | Anthocyanin |
| | | | | | | | antagonist |
| TAS4a | 3,708 | 0.76 | 0.21 | 14 | −1.84 | 0.20 | Anthocyanin repress |
| TAS4b | 300 | −0.55 | 0.24 | 25 | −1.17 | 0.28 | Anthocyanin repress |
| TAS4c | 35.6 | −2.31 | 0.11 | n.d. | | | Anthocyanin repress |
| MYBA6, TAS4 | 1.3 | −0.52 | 0.61 | 20 | −2.72 | 0.03 | Anthocyanin |
| target PHAS | | | | | | | biosynthesis |
| MYBA7, TAS4 | 0.9 | n.a. | | 2 | −5.18 | 0.19 | Anthocyanin |
| target PHAS | | | | | | | biosynthesis |
| Tl-deoxy-D- | | | | 90 | −2.56 | 10⁻⁴ | isoprenoid |
| xylulose-5- | | | | | | | biosynthesis; binds |
| phosphate | | | | | | | suppressor HC-Pro |
| synthase, DXS | | | | | | | 00s0218g00110 |
| SAMDC | | | | 2759 | −1.23 | 0.02 | Me-anthranilate synth, |
| | | | | | | | viral methylation |
| | | | | | | | 01s0010g00990 |
| Mg chelatases | | | | 2744 | −1.13 | 0.02 | 05s0102g00310/GUN4 |
| | | | | 22.7 k | −0.98 | 0.04 | 08s0007g08540 |
| Mg dechelatase | | | | 1766 | 1.43 | 0.002 | 02s0025g04660 |
| CaM-L37-like | | | | 358 | 2.29 | 0.002 | 18s0001gll830 |
| CaM-L37-like | | | | 27 | 2.96 | 0.03 | 05s0102g00450 |
| CaM-L37-like | | | | 307 | 1.17 | 0.05 | 14s0006g01400 |
| DICER2, PHAS | | | | 2094 | 1.90 | 0.007 | Viral siRNA biosynth |
| AGO7 | | | | 2 | 3.90 | 0.30 | Viral siRNA activity |
| AGO7-like | | | | 14 | 1.25 | 0.39 | Viral siRNA activity |
| AGO5 | | | | 1 | 2.46 | 0.66 | Viral siRNA activity |
| GRBV C3 mm(−) | 1.6 | | | 11 | 3.00 | 0.08 | Me-anthraniloyal T- |
| | | | | | | | ase 02s0033g01070 |
| GRBV V1-mm | 1.1 | | | 1964 | 0.51 | 0.31 | Anthocyan permase |
| | | | | | | | 16s0050g00930 |
| GRBV C2 mm(−) | 0.9 | | | 570 | 0.17 | 0.73 | SGS3, PHAS locus |
| | | | | | | | 07s0130g00190 |
| GRBV V3-mm | 0.2 | | | 39 | 0.62 | 0.53 | Ankyrin |
| | | | | | | | 05s0029g01430 |
| GRBV V1 | 0.2 | | | 2327 | 0.30 | 0.56 | NADP-malic enzym |
| | | | | | | | lls0016g03210 |
| GRBV C3(−) | 0.15 | | | 3093 | −0.38 | 0.45 | Oxygen-evol PSBQ |
| | | | | | | | 00s0904g00010 |
| GRBV V1 | 0.16 | | | 91 | 0.23 | 0.75 | CHS isomerase3 |
| | | | | | | | 19s0014g00100 |
| GRBV V3-mm | 0.15 | | | 1511 | −0.68 | 0.20 | Ca²⁺ sensor |
| | | | | | | | 17s0000g04490 |

n.d.: not detected; n.a.: not analyzed
^{†}targets not yet validated functionally
^{¶}concordant expression with miRNA effector LFC
\hat{ }other family members have star species dominant

Consistent with chlorosis [133] as a GRBV symptom, RNA-seq differential expression [134] (DE) analysis of asymptomatic early-season GRBV-infected leaves found 721 genes affected (533 up, 188 down). Photosynthesis was the most-significantly over-represented

metabolic process (p<10⁻⁶; Wilcoxon Rank Sum test, Benjamini-Hochberg adjusted) [135]. Of note is significant up-regulation of ‘stay green’ magnesium dechelatase VIT_02s0025g04660 involved in chlorophyll degradation, and significant down regulation of two Mg-chelatases (Table IV, above).

Galactinol oligosaccharide synthase genes involved in oxidative and stress adaptations [136] were strongly up-regulated, as were genes for pectate lyases involved in cell wall degradation and pectin methylesterase inhibitor VIT_16s0022g00960 (LFC 3.65,p<10⁻⁵) previously shown to be up-regulated in GRBV-infected berries [37]. Other pathways showed mixed induction and down-regulations such as secondary phenolics (Phe ammonia lyases and proteolytic regulator KFB-PAK VIT_08s0007g07120 up, flavonoid 3′,5′-hydroxylase VIT_06s0009g02860 down) and terpenoid metabolism including biosynthetic enzymes germacreneD synthase (VIT_19s0014g01070, LFC—3.97, p<10⁻¹⁰) and 1-deoxy-D-xylulose-5-phosphate synthase (DXS/VIT_00s0218g00110) significantly down-regulated (Table IV). Isoprenoids are known to be altered in virus-infected leaves [16], and recently a physical interaction between Potyvirus suppressor protein HC-Pro and tobacco DXS involved in isoprenoid biosynthesis was shown [137].

Another intriguing observation is the most significantly DE gene, and a homologue ranked 47th most significantly DE, are calmodulin (CaM)-binding IQ-Domain67-like proteins (VIT_00s1881g00010; LFC 5.24, p<10⁻¹⁰, VIT_00s0366g00010; LFC 6.70, p<0.0003) that likely function as hubs in cellular calcium signaling [138]. Potyvirus HC-Pro induces expression of and binds regulator-of-gene-silencing rgs-CaM in tobacco [139], and rgs-CaM has recently been shown to function as an immune receptor by promoting hypersensitive responses such as Ca²⁺ fluxes, production of reactive oxygen species, and salicylic acid-mediated degradation of Cucumber mosaic virus suppressor 2b by autophagy [140]. rgsCaM prevents TEV HcPro and Cucumber mosaic virus suppressor 2b from binding to dsRNAs/siRNAs and reduce the suppressor protein stability by autophagy, resulting in a more potent RNAi defense against viral infection. rgsCaM over-expressing lines were less susceptible to the virus [141]. Interestingly, TGMV AC2 induces a calmodulin-like protein Nb-rgsCaM [41] and over-expression of rgsCaM leads to an increase in viral DNA load. rgsCaM self-interaction was observed in cytoplasm while interaction with TGMV AC2 sequestered rgsCaM to the nucleus. It was speculated that AC2-mediated localization of rgsCaM to the nucleus is the likely mechanism evolved by TGMV to evade degradation of AC2 by autophagy and thereby effectively suppress the plant defense mechanism. Three rgs-CaM homologs (CaM-L37-like) were found in grape that are significantly up-regulated in GRBV-infected asymptomatic field samples (Table IV). A similar mechanism adapted by GRBV to evade rgsCaM-mediated autophagy cannot be discounted.

Concordant with this working hypothesis and prior evidence (Table II) that GRBV disease etiology is associated with host PTGS processes including miR828/TAS4/MYBA6/7 regulon, the inventors found miR828 pri-MIRNA and TAS4bc are down-regulated in the RNAseq analysis of asymptomatic field samples from Jacksonville OR, 2018 while MYBA6/7 expression is significantly down-regulated and other MYB targets of miR828 up-regulated (data not shown; Table II). VvDXS and VvSAMDC are significantly down-regulated in asymptomatic GRBV-infected field samples, whereas PHAS locus DICER2 [103], ZIPPY/AG07-like, and AGO5 involved in miRNA/phasiRNA biogenesis and/or viral resistance [142-145] are (significantly) up-regulated (Table IV), as are homologous autophagy effectors VIT_02s0154g00390 and VIT_12s0059g00660/APG8d (LFC ˜1.22, p=0.06). RNASeq is used on symptomatic GRBV-(and GLRaV2/3 and grapevine fleck virus) infected field samples, to establish by statistical power of biological replicates across time, space, and genotype whether these GRBV associations to identify the functionally conserved sequences among different virus families and conclusive evidence for mechanisms underlying GRBV disease etiology.

Of significance is the finding that abundant GRBV vsiRNA C3 mm (−) in symptomatic infected leaves may alter expression of Me-anthraniloyal transferase (AMAT) VIT_02s0033g01070, a gene that synthesizes methyl anthranilate using SAM as substrate. SAMDC down regulation by GRBV would increase SAM substrate for production of Me-anthranilate by strongly elevated AMAT expression (Table IV). Me-anthranilate is volatile and has a fruity/musky smell used in the food and perfume industries, and more importantly is known to attract insect vectors [146, 147]. These results demonstrate GRBV host symptoms serve as visual (anthocyanins) and/or olfactory cues to arthropod vectors. These results can help explain the observed (Table III) rapid spread of the virus threatening the industry.

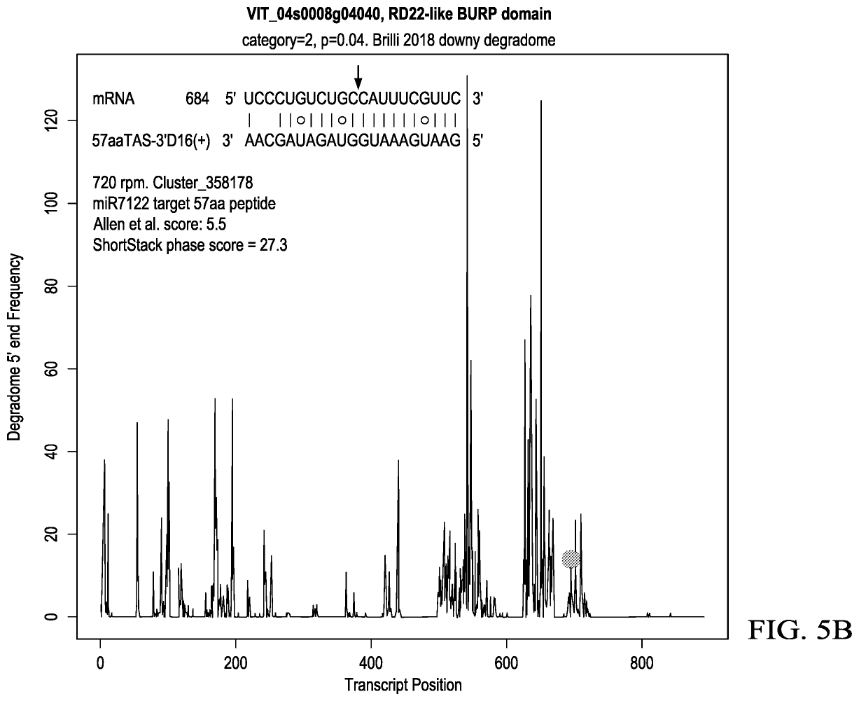

The in-process RNA seq data and sRNA analysis by PhaseTank [148] of symptomatic GRBV sample datasets can provide conclusive evidence, since the inventors (Table IV) and others' (Table II) data show GRBV likely targets the miR828/TAS4/MYB auto-regulatory loop. The inventors observed highly significant upregulation of miR2950 in the GRBV symptomatic samples. VIT_07s0151g00190, VIT_07s0151g00110, and VIT_07s0151g00250 Chlorophyllase gene expressions were predicted to be post-transcriptionally regulated by the grapevine-specific miRNA miR2950 [132]. An inverse relationship between miR2950 and a predicted target gene was found exclusively in grapevine virus B (GVB)-infected plants [149]. The inventors observed a similar inverse regulation of miR2950 (LFC 2.94; p<10⁻⁷) and VIT_07s0151g00110 (Chlorophyllase homolog; LFC −2.06; p=0.23) in this study of GRBV infected samples. miR398c also displayed significant upregulation in infected samples and its target Blue Copper-Binding Protein gene was downregulated (albeit not significantly) in asymptomatic field samples. miR2950 and miR398 of cotton have been claimed to target the genome of monopartite geminivirus Cotton leaf curl Multan virus [150], whereas overexpression of MIR2950 and MIR398 conferred resistance to the virus [151]. A degradome analysis did not find any evidence for these or other grape miRNAs to target GRBV genome (data not shown), thus the role of the above miRNAs is likely limited to host gene targets in GRBV-infected plants. Similar inverse relationships were observed between DE miR3632/482-L, miR3624a,b, miR169g*, miR169x, miR530b-isomiR, novel miR13/12/14, miR156b*, miR403a, miR7122 target TAS-14s0081g00100 D16(+), which targets a BURP domain-containing RD22 PHAS locus and potentially several homologs (FIG. 5B), and their respective established or predicted target effectors (Table IV). Many of the targets of the DE-expressed miRNAs are known to be involved in regulating plant pathogen resistance genes and host silencing machinery, supporting the rationale for identifying DE expressed miRNAs and their respective targets in GRBV infected samples. The DE of miRNA targets are further validated with RNAseq data analysis to build a compelling and testable mechanistic model for GRBV infection. FIG. 5. CleaveLand [159] T plots of grapevine degradome evidence for A) miR7122 slicing of TAS-14s0081g00100 PHAS locus [148] to generate 3′D16(+) tasiRNA. B) RD22 BURP domain homologue of genes listed in Table IV being sliced by novel TAS-14s0081g00100 tasiRNA3′D16(+) derived from miR7122.

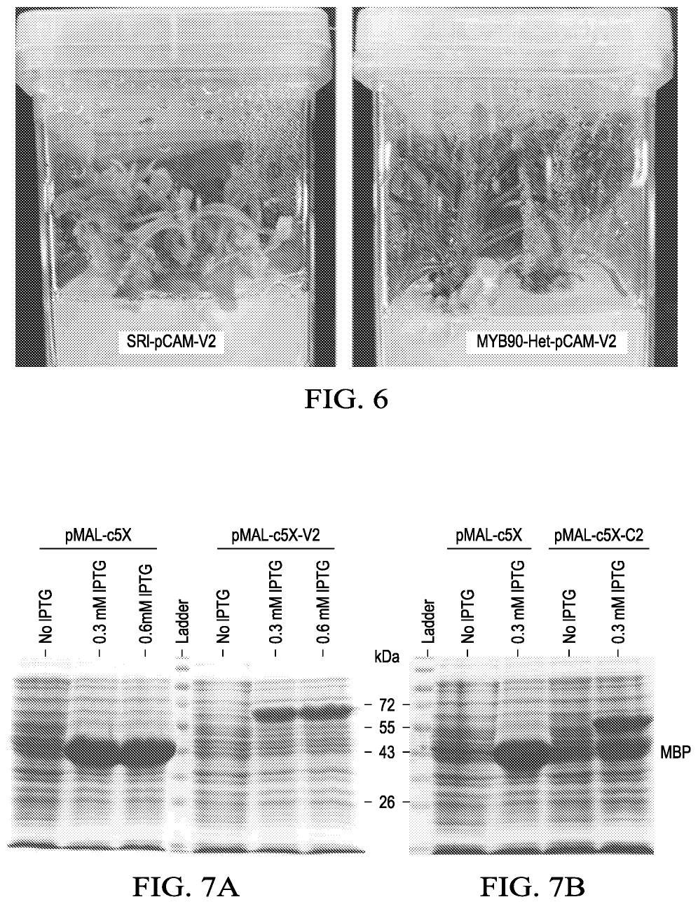

Based on the model that GRBV suppressor proteins target the MIR828-TAS4-1MYBA5/6/7 autoregulatory loop, a super-transformation experiment with GRBV suppressor protein C2- and V2-expressing binary constructs inoculated into a transgenic tobacco line that overexpresses the Arabidopsis target of TAS4 siRNA: AtMYB90/PRODUCTION_OF-ANTHOCYANIN_PIGMENT2 [152] can be made. Axenic tissue-cultured control, hemizygous and homozygous transgenic plants were established that have been super-transformed with empty binary vector-pCAMBIA2301 or with binary vector harboring the GRBV ORFs C2/V2 (pCAM-C2/pCAM-V2). The leaf discs transformed were selected on shooting media containing cefotaxime 250 mg/L and kanamycin 100 mg/L. The regenerated shoots have been established on rooting media (FIG. 6; representative picture of regenerated shoots upon pCAM-V2 super-transformation).

FIG. 6. Control (SRI, left) and heterozygous AtMYB90 tobacco plants (right) super-transformed with pCAM-V2 in rooting medium. The results from multiple years of field sampling has helped decipher the DE miRNAs in GRBV-infected samples. The inventors have also identified the inverse regulation of targets in a preliminary RNAseq dataset. The tobacco transgenics are established and ready for molecular characterization. The significance is synergy with other results herein, as shown in sRNA and total RNA blots probed for the putative host target miRNAs in C2 and V2- over-expressing lines will provide evidence of sufficiency for hypothesized V2 and C2 functions to silence anthocyanin biosynthesis mediated by host phasiRNAs. A concordance in expression profiles of targets was established in over-expression lines with that of field sample sequencing data which could independently validate the sRNA targets of GRBV suppressor proteins.

Example 3. Identify the Host Grapevine Targets of GRBV Suppressor Proteins C2 and V2.

To understand if the mechanism of silencing suppression is by binding miRNA/siRNA, the suppressor proteins C2 and V2 using pMAL™ Protein Fusion & Purification System (New England Biolabs) are produced. PCR-amplified GRBV C2 and V2 genes were inserted as blunt end fragments in the 5′ end and with SbfI restriction site in the 3′ end which were cloned into the pMAL-c5X vector digested with XmnI and SbfI. The clones were confirmed by restriction digestion (data not shown) and Sanger sequencing. The clones were re-transformed into E. coli strain ER2523 (NEB Express) for protein expression. As a pilot experiment, cells were grown to 0.5 OD at 37° C., induced with 0.3 mM IPTG for four hr. The cells were re-suspended and run on an SDS-PAGE gel. The maltose binding protein (MBP) was observed at 42.5 kDa upon inducing cells transformed with pMAL empty vector (FIG. 7A, 7B). The pMAL-V2 fusion protein was observed at 61.5 kDa (FIG. 7A) and pMAL-C2 protein at 59.4 kDa (FIG. 7B) as expected. However, in addition to the pMAL-C2 fusion protein, MBP (a highly stable protein) was also observed at 42.5 kDa in pMAL-C2 induced cells (FIG. 7B). This is likely due to proteolysis of the fusion protein. FIG. 7. pMAL protein expression 7A) pMAL-c5x-V2, 7B) pMAL-c5x-C2

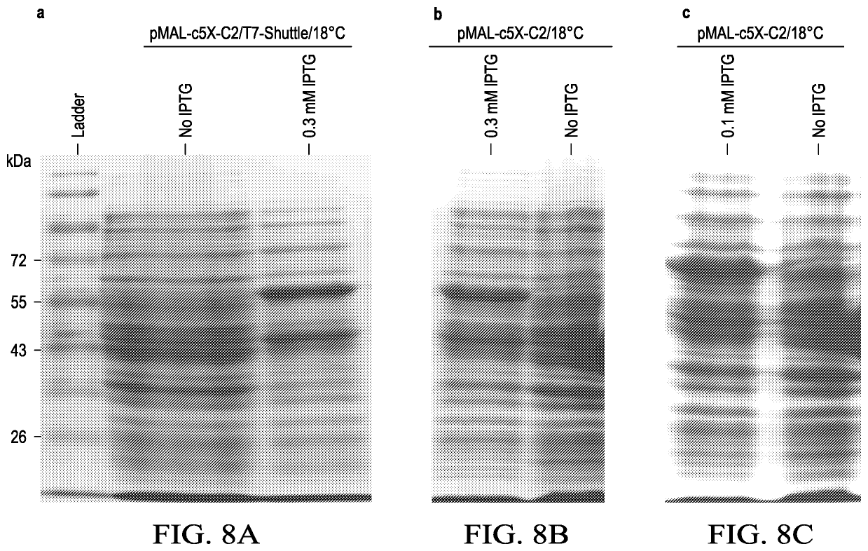

To overcome this limitation, pMAL-C2 vector were retransformed in a protease-deficient strain (T7 Shuffle). The cells were grown at 37° C., induced with 0.3 mM IPTG for four hr and checked for induction using SDS-PAGE. Proteolysis of induced protein was observed (data not shown). To reduce the proteolysis pMAL-C2 in T7-shuttle were induced and in NEB Express cells at 18° C. for 18 hours with 0.3 mM IPTG. Proteolysis of induced protein was observed in T7-shuttle cells (FIG. 8A) as well as in NEB-Express cells (FIG. 8B) by the presence of an MBP-sized band of ˜42.5 kDa. However, the proteolysis was less in NEB-Express (FIG. 8B) in comparison to T7-shuttle cells (FIG. 8A). Hence, pMAL-C2 in NEB-Express was induced with a lower 0.1 mM IPTG concentration at 18° C. for 18 hours. The proteolysis of fusion protein was reduced (FIG. 8C) and hence large scale induction will be performed using the above condition. The induced protein will be purified and used for ss- and dsDNA binding assays by electrophoretic mobility shift assay [153] or solution-based in vitro binding assays with an anti-MBP antibody or amylose beads to capture the C2:MBP and V2:MBP bound to nucleic acids (selected from Table IV) as the next step.

FIG. 8. pMAL-c5x-C2 protein expression at 18° C. for 18 hours a) T7 shuttle with 0.3 mM IPTG b) NEB Express with 0.3 mM IPTG c) NEB Express with 0.1 mM IPTG

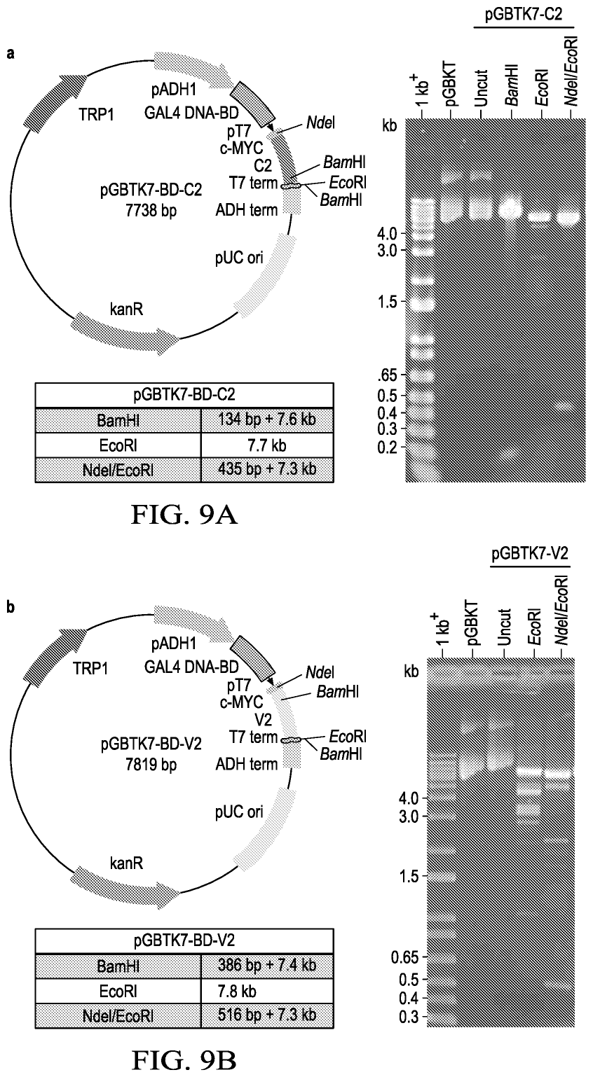

A yeast two-hybrid (Y2H) screen can be used as an unbiased alternative approach to discover host proteins that bind physically to GRBV C2 and V2. Towards this objective suppressor genes C2 and V2 were cloned in a bait vector pGBTK7-BD. GRBV C2 and V2 were PCR amplified with primers flanking NdeI and EcoRI restriction sites and were introduced into the corresponding sites of pGBTK7-BD vector. The clones were confirmed by restriction analysis (FIG. 9A, 9B) and sequencing. A grape cDNA library using Mate & Plate library system (Takara) can be made to identify the targets of GRBV C2 and V2 in grape cDNA library by Y2H screening. Based on the transient co-infiltration analyses, co-expression of C2+V2 had higher suppression activity (FIGS. 3A and 3B). Therefore it is of interest to test C2-V2 viral protein interactions by cloning C2 and V2 in prey vector.

Cloning of V2 and C2 in expression vectors and the induction conditions were standardized and can be used for large scale protein purification and binding assays. The inventors cloned the bait vectors for use in Y2H assay with grape cDNA library as the next step.

Example 4: Initiate transgenic grapevine experiments to test disease resistance of transgenic grape expressing hairpin silencers directed to GRBV suppressor protein transcripts.

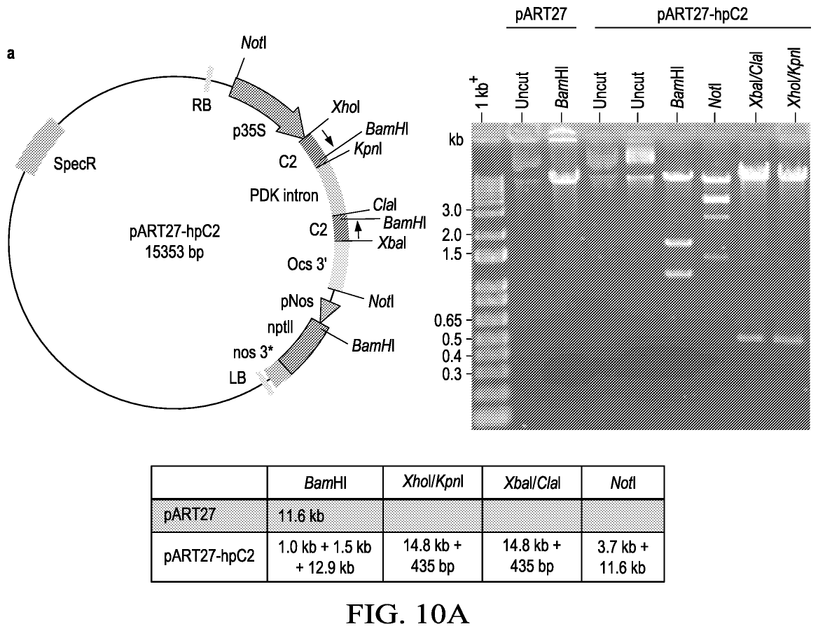

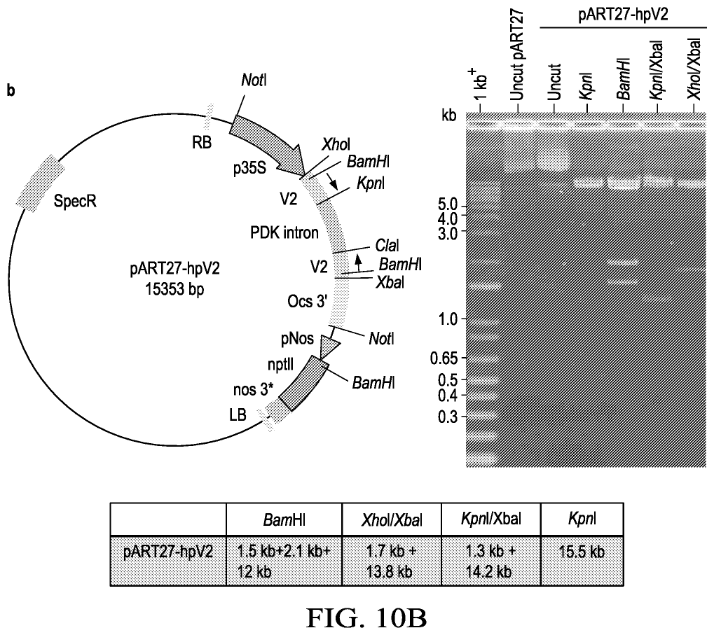

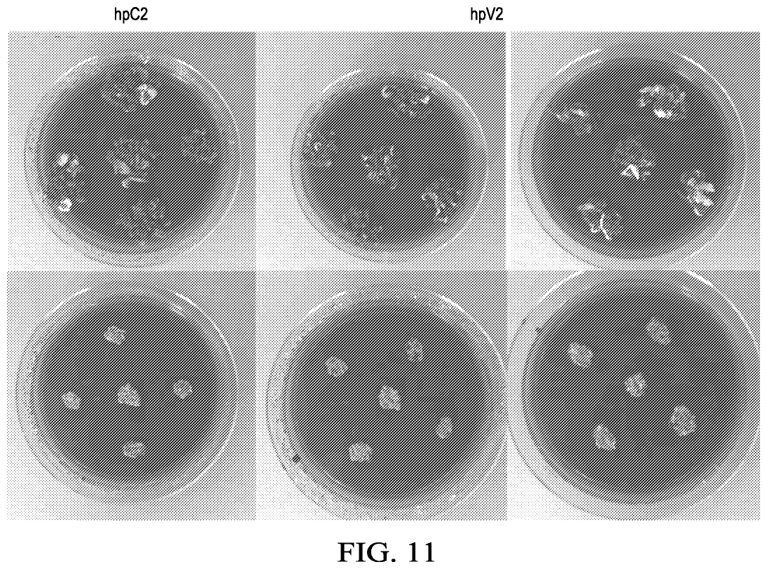

Several reports have demonstrated that PTGS of viral suppressor proteins is an effective strategy to engineer viral resistance. Construct hpRNA vectors targeting GRBV C2 and V2 genes can be made. The inventors confirmed the C2 and V2 genes are highly conserved across 93 known GRBV isolates by multiple sequence alignment (data not shown) [2]. Towards making the hpRNA construct, PCR amplified C2 and V2 genes were engineered by introducing XhoI and KpnI sites in the primers and cloning the digested PCR product in the corresponding sites of pHANNIBAL vector [154] to obtain the sense orientation clone (pHANNIBAL-C2/pHANNIBAL-V2) which was confirmed by restriction digestion analysis. To clone the antisense arm of the hairpin vector, the inventors PCR-amplified C2 and V2 with primers flanked by ClaI and XbaI restriction sites and cloned them in the corresponding sites of pHANNIBAL-C2/pHANNIBAL-V2 to obtain the hpRNA vector pHANNIBAL-hpC2 or pHANNIBAL-hpV2. The clones were confirmed by restriction digestion analysis and sequencing. The hpRNA gene cassette comprising the hpC2 or hpV2 was excised as a NotI fragment and cloned in the Non site of T-DNA binary vector pART27 [139], which harbors the neomycin phosphotransferaseII gene as the plant transformation marker under the nopaline synthase promoter and terminator. The clones were confirmed by restriction analysis (FIG. 10A, 10B). The binary vector was mobilized into A. tumefaciens strain EHA105 by electroporation and confirmed by PCR. The recombinant DNA vector strains were shipped under USDA-BRS permit #19-330-112m to Cooperator Tricoli for 101-14 transformation in March 2020. Three independent transformations were initiated with pART27-empty vector, pART27-hpC2 and pART27-hpV2, respectively. The current status of transformation events is listed in Table V. Regeneration of plantlets is in process under duly executed fee-for-service contract #C15297 (FIG. 11; representative picture of regenerated grape shoots).

FIG. 9. Restriction enzyme digestion 9a) pGBTK7-BD-C2, 9b) pGBTK7-BD-V2 *EcoRI digestion displayed star activity.

FIG. 10. Restriction enzyme digestion 10a) pART27-hpC2, 10b) pART27-hpV2 *NotI digestion displayed star activity.

FIG. 11. Current status of somatic embryogenesis to regenerate transgenic plantlets from two rounds of Agrobacterium-mediated anther-derived callus transformation and selection.

TABLE V
Status of grape transformation

| Cultivar | Agro Strain | PL Construct Code | PTF Code | Plant Selection | Explant |
|---|---|---|---|---|---|
| 101-14 | EHA105 Gelvin | pART27 | AT20046 | kanamycin | immature embryos |
| 101-14 | EHA105 Gelvin | pART27 hpC2 | AT20047 | kanamycin | immature embryos |
| 101-14 | EHA105 Gelvin | pART27 hpV2 | AT20048 | kanamycin | immature embryos |
| 101-14 | EHA105 Gelvin | pART27 | AT20046 | kanamycin | immature embryos |
| 101-14 | EHA105 Gelvin | pART27 hpC2 | AT20047 | kanamycin | immature embryos |
| 101-14 | EHA105 Gelvin | pART27 hpV2 | AT20048 | kanamycin | immature embryos |
| 101-14 | EHA105 Gelvin | pART27 | AT20046 | kanamycin | immature embryos |
| 101-14 | EHA105 Gelvin | pART27 hpC2 | AT20047 | kanamycin | immature embryos |
| 101-14 | EHA105 Gelvin | pART27 hpV2 | AT20048 | kanamycin | immature embryos |

Example V: Evaluate in early 2021 hpRNA transgenic grapevine for GRBV resistance/susceptibility.

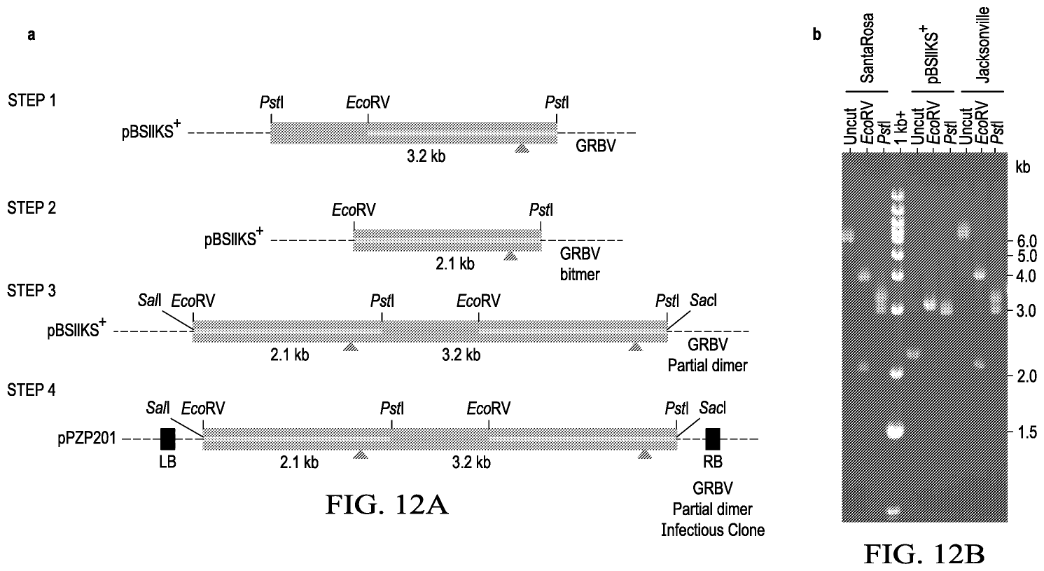

The ability to infect and invade is a fundamental requirement for a successful pathogen. To test the transgenic plants from Example IV for disease resistance the inventors initiated cloning of GRBV viral clones for agroinfection [10]. GRBV full length genomic sequence of 3.2 kb was cloned into pBSII-KS⁺ to yield pBS-GRBV vector following rolling circle amplification (RCA) (GE Healthcare) and restriction digestion with PstI enzyme of RCA product from field-infected grape leaf samples from Santa Rosa and Jacksonville (FIG. 12A, STEP 1). The clones were confirmed by restriction digestion (FIG. 12b) and were validated using Sanger sequencing. A partial dimer, also known as a ‘bitmer’ or partial tandem repeat construct [10], comprised of a tandem duplication of a portion of the GRBV genome containing the common region (CR), is constructed such that sequences for two duplicated CR stem loop origins of replication flank the remainder of the genome and adjoining sequences as shown in FIG. 12A. STEPs 2-4 complete the construction of a binary vector for agroinfection assay with greenhouse-grown grapevine rootstock 101-14 plants.

FIGS. 12A and 12B. Cloning of GRBV viral partial dimer constructs for agroinfection functional assays. (12A) Schematic representation of partial dimer cloning. Triangles refer to tandem repeats of the common region (CR) that recombine in host to release an infectious viral genome nicked by viral replicase and amplified by rolling circle replication [10]. (12B) Restriction enzyme digestion of full length viral clone in pBSILKS⁺. Hairpin vector construction and initiation of three independent transformations for each binary vector was conducted. Construction of partial dimer viral clone in binary vector for agroinfection assay has also been conducted.

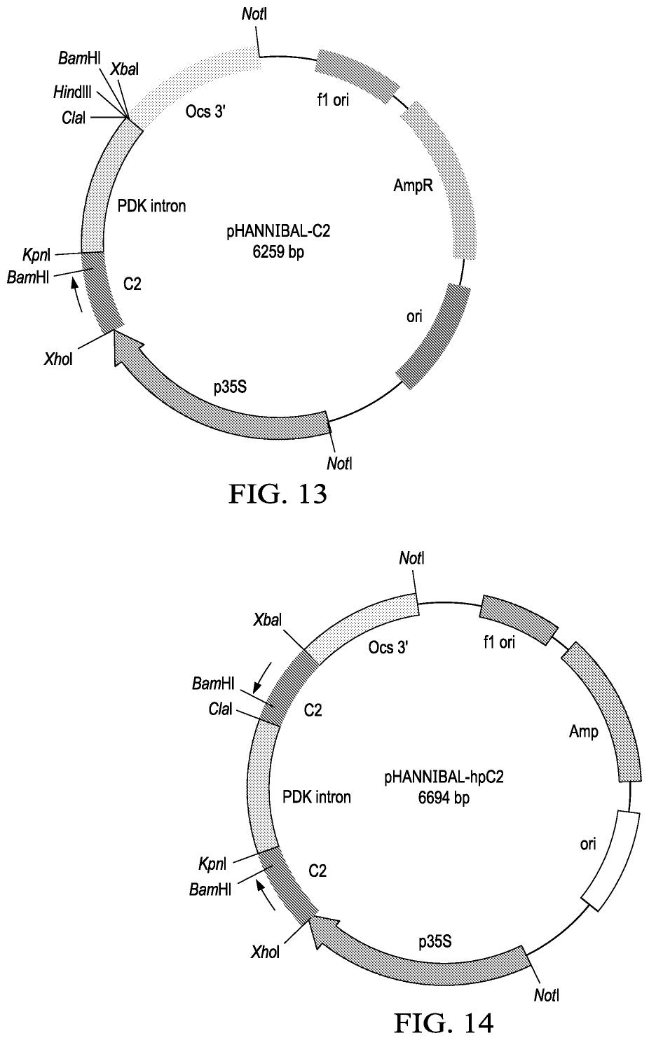

FIG. 13 a pHANNIBAL-C2 vector of the present invention with a C2 insert, PDK insert under the control of a p35S promoter, fl ori, Amp resistance, bacterial ori and Ocs 3′. FIG. 14 a pHANNIBAL-hpC2 vector of the present invention with two C2 inserts in opposite directions, PDK insert under the control of a p35S promoter, fl ori, Amp resistance, bacterial ori and Ocs 3′.

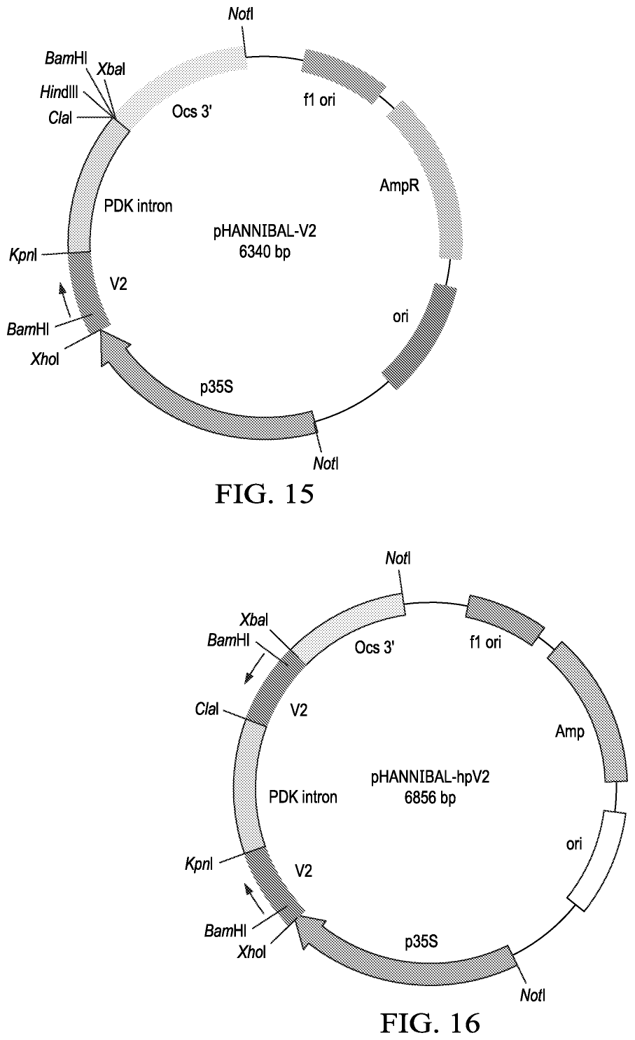

FIG. 15 a pHANNIBAL-V2 vector of the present invention with a V2 insert, PDK insert under the control of a p35S promoter, fl ori, Amp resistance, bacterial ori and Ocs 3′. FIG. 16 a pHANNIBAL-hpV2 vector of the present invention with two V2 inserts in opposite directions, PDK insert under the control of a p35S promoter, fl ori, Amp resistance, bacterial ori and Ocs 3′.

Conclusions. The inventors have identified C2 and V2 as suppressors of PTGS. The inventors completed sRNA and RNAseq library sequencing and analysis of samples collected from the field in 2018. mRNA sequencing and analysis of libraries can be made from 2019 field samples. The inventors cloned the suppressor proteins in pMAL-c5X vector and in pGBTK7-BD vector for protein purification and Y2H assay, respectively. The inventors completed the binary vector cloning of hpRNA vector targeting the GRBV suppressor proteins. This comprehensive study sought to understand the viral gene functions and effects on host physiology and molecular mechanisms of genomic regulation to deploy multiple cogent strategies for mitigating red blotch disease. Towards this the inventors characterized the viral proteins that suppress plant defense mechanism, and developed transgenics to target the incoming virus. Targeting the viral transcripts inhibits viral replication and thereby inhibit the disease spread. The anti-viral siRNAs can operate systemically by moving through vasculature, raising prospects of genetic engineering of grapevine rootstocks for GRBV resistance in non-genetically modified organism (GMO) scions.

By way of explanation, and in no way a limitation of the present invention, the inventors hypothesize the viral suppressor protein(s) of GRBV specifically, and likely other grapevine viruses like Fan Leaf and Leaf-Roll-associated Virus, interfere with the anthocyanin regulatory pathways and result in uncontrolled anthocyanin accumulation in vegetative tissues, thus serving as a visual cue for feeding by the assumed arthropod vector capable of transmitting the viruses. The inventors have identified the GRBV viral suppressor proteins as GRBV genes C2 and V2. The present invention includes the expression of ‘knockdown’ hairpin gene constructs using recombinant plasmids pART27 and pHANNIBAL directed against GRBV V2 and C2 in stably transformed transgenic grapevine plants. This creates an innate immunity GRBV host resistance by using endogenous RNA interference mechanisms of the host plant directed to target and silence infecting GRBV sequences.

Sequences.

| | | SEQ ID |
|---|---|---|
| Name | Sequence | NO: |
| C2 | >pART27_hpC2_sequence | 1 |
| | TCGACATCTTGCTGCGTTCGGATATTTTCGTGGAGTTCCCGCCACAGACCCG | |
| | GATTGAAGGCGAGATCCAGCAACTCGCGCCAGATCATCCTGTGACGGAAC | |
| | TTTGGCGCGTGATGACTGGCCAGGACGTCGGCCGAAAGAGCGACAAGCAG | |
| | ATCACGATTTTCGACAGCGTCGGATTTGCGATCGAGGATTTTTCGGCGCTG | |
| | CGCTACGTCCGCGACCGCGTTGAGGGATCAAGCCACAGCAGCCCACTCGA | |
| | CCTTCTAGCCGACCCAGACGAGCCAAGGGATCTTTTTGGAATGCTGCTCCG | |
| | TCGTCAGGCTTTCCGACGTTTGGGTGGTTGAACAGAAGTCATTATCGTACG | |
| | GAATGCCAGCACTCCCGAGGGGAACCCTGTGGTTGGCATGCACATACAAA | |
| | TGGACGAACGGATAAACCTTTTCACGCCCTTTTAAATATCCGTTATTCTAAT | |
| | AAACGCTCTTTTCTCTTAGGTTTACCCGCCAATATATCCTGTCAAACACTGA | |
| | TAGTTTAAACTGAAGGCGGGAAACGACAATCTGATCATGAGCGGAGAATT | |
| | AAGGGAGTCACGTTATGACCCCCGCCGATGACGCGGGACAAGCCGTTTTAC | |
| | GTTTGGAACTGACAGAACCGCAACGATTGAAGGAGCCACTCAGCCCCAAT | |
| | ACGCAAACCGCCTCTCCCCGCGCGTTGGCCGATTCATTAATGCAGCTGGCA | |
| | CGACAGGTTTCCCGACTGGAAAGCGGGCAGTGAGCGCAACGCAATTAATG | |
| | TGAGTTAGCTCACTCATTAGGCACCCCAGGCTTTACACTTTATGCTTCCGGC | |
| | TCGTATGTTGTGTGGAATTGTGAGCGGATAACAATTTCACACAGGAAACAG | |
| | CTATGACCATGATTACGCCAAGCTATTTAGGTGACACTATAGAATACTCAA | |
| | GCTATGCATCCAACGCGTTGGGAGCTCTCCCATATCGACCTGCAGGCGGCC | |
| | GCTCGACGAATTAATTCCAATCCCACAAAAATCTGAGCTTAACAGCACAGT | |
| | TGCTCCTCTCAGAGCAGAATCGGGTATTCAACACCCTCATATCAACTACTA | |
| | CGTTGTGTATAACGGTCCACATGCCGGTATATACGATGACTGGGGTTGTAC | |
| | AAAGGCGGCAACAAACGGCGTTCCCGGAGTTGCACACAAGAAATTTGCCA | |
| | CTATTACAGAGGCAAGAGCAGCAGCTGACGCGTACACAACAAGTCAGCAA | |
| | ACAGACAGGTTGAACTTCATCCCCAAAGGAGAAGCTCAACTCAAGCCCAA | |
| | GAGCTTTGCTAAGGCCCTAACAAGCCCACCAAAGCAAAAAGCCCACTGGC | |
| | TCACGCTAGGAACCAAAAGGCCCAGCAGTGATCCAGCCCCAAAAGAGATC | |
| | TCCTTTGCCCCGGAGATTACAATGGACGATTTCCTCTATCTTTACGATCTAG | |
| | GAAGGAAGTTCGAAGGTGAAGGTGACGACACTATGTTCACCACTGATAAT | |
| | GAGAAGGTTAGCCTCTTCAATTTCAGAAAGAATGCTGACCCACAGATGGTT | |
| | AGAGAGGCCTACGCAGCAGGTCTCATCAAGACGATCTACCCGAGTAACAA | |
| | TCTCCAGGAGATCAAATACCTTCCCAAGAAGGTTAAAGATGCAGTCAAAA | |
| | GATTCAGGACTAATTGCATCAAGAACACAGAGAAAGACATATTTCTCAAG | |
| | ATCAGAAGTACTATTCCAGTATGGACGATTCAAGGCTTGCTTCATAAACCA | |
| | AGGCAAGTAATAGAGATTGGAGTCTCTAAAAAGGTAGTTCCTACTGAATCT | |
| | AAGGCCATGCATGGAGTCTAAGATTCAAATCGAGGATCTAACAGAACTCG | |
| | CCGTGAAGACTGGCGAACAGTTCATACAGAGTCTTTTACGACTCAATGACA | |
| | AGAAGAAAATCTTCGTCAACATGGTGGAGCACGACACTCTGGTCTACTCCA | |
| | AAAATGTCAAAGATACAGTCTCAGAAGACCAAAGGGCTATTGAGACTTTTC | |
| | AACAAAGGATAATTTCGGGAAACCTCCTCGGATTCCATTGCCCAGCTATCT | |
| | GTCACTTCATCGAAAGGACAGTAGAAAAGGAAGGTGGCTCCTACAAATGC | |
| | CATCATTGCGATAAAGGAAAGGCTATCATTCAAGATCTCTCTGCCGACAGT | |
| | GGTCCCAAAGATGGACCCCCACCCACGAGGAGCATCGTGGAAAAAGAAGA | |
| | CGTTCCAACCACGTCTTCAAAGCAAGTGGATTGATGTGACATCTCCACTGA | |
| | CGTAAGGGATGACGCACAATCCCACTATCCTTCGCAAGACCCTTCCTCTAT | |
| | ATAAGGAAGTTCATTTCATTTGGAGAGGACACGATGTTGAAAGAATATATA | |
| | ACACTGACGATGAGCTCAACAATGCAGGAAGCGACGCACCGAAGGAGGAA | |
| | GAGCCTGGTGATTGAAGGACCATCGAAGACGGGTAAAACGCAATGGGCTA | |
| | TATCATTGGGAATACATAACTACTGGTGTGAATCAGTAGACTTCAGTCTAT | |
| | ACATAGACGACGCTTTGTACAATATTATTGACGATATACCATTCCAATATTT | |
| | GCCTTGTAAAAAAGCCTTATTAGGTTGTCAATTCAATTACATTGCAAATGA | |
| | AAAATATCGTCCAAAGCGGAAAATCAAAGGAGGGATCCCATCAATTGTTC | |
| | TTTGTAATCCAGATAACTCTTACTATGATGCAATCACCAATTGGCATGCAA | |
| | CATTCAAGCCGTGGGCTGAAGAAAACATTATCTTTGTAAAAATTGATAGAC | |
| | CGTTGTACTGATCTTTTTTCCTTTTAGTATAAAATAGTTAAGTGATGTTAAT | |
| | TAGTATGATTATAATAATATAGTTGTTATAATTGTGAAAAAATAATTTATA | |
| | AATATATTGTTTACATAAACAACATAGTAATGTAAAAAAATATGACAAGTG | |
| | ATGTGTAAGACGAAGAAGATAAAAGTTGAGAGTAAGTATATTATTTTTAAT | |
| | GAATTTGATCGAACATGTAAGATGATATACTAGCATTAATATTTGTTTTAAT | |
| | CATAATAGTAATTCTAGCTGGTTTGATGAATTAAATATCAATGATAAAATA | |
| | CTATAGTAAAAATAAGAATAAATAAATTAAAATAATATTTTTTTATGATTA | |
| | ATAGTTTATTATATAATTAAATATCTATACCATTACTAAATATTTTAGTTTA | |
| | AAAGTTAATAAATATTTTGTTAGAAATTCCAATCTGCTTGTAATTTATCAAT | |
| | AAACAAAATATTAAATAACAAGCTAAAGTAACAAATAATATCAAACTAAT | |
| | AGAAACAGTAATCTAATGTAACAAAACATAATCTAATGCTAATATAACAA | |
| | AGCGCAAGATCTATCATTTTATATAGTATTATTTTCAATCAACATTCTTATT | |
| | AATTTCTAAATAATACTTGTAGTTTTATTAACTTCTAAATGGATTGACTATT | |
| | AATTAAATGAATTAGTCGAACATGAATAAACAAGGTAACATGATAGATCA | |
| | TGTCATTGTGTTATCATTGATCTTACATTTGGATTGTCAGTACAACGGTCTA | |
| | TCAATTTTTACAAAGATAATGTTTTCTTCAGCCCACGGCTTGAATGTTGCAT | |
| | GCCAATTGGTGATTGCATCATAGTAAGAGTTATCTGGATTACAAAGAACAA | |
| | TTGATGGGATCCCTCCTTTGATTTTCCGCTTTGGACGATATTTTTCATTTGCA | |
| | ATGTAATTGAATTGACAACCTAATAAGGCTTTTTTACAAGGCAAATATTGG | |
| | AATGGTATATCGTCAATAATATTGTACAAAGCGTCGTCTATGTATAGACTG | |
| | AAGTCTACTGATTCACACCAGTAGTTATGTATTCCCAATGATATAGCCCATT | |
| | GCGTTTTACCCGTCTTCGATGGTCCTTCAATCACCAGGCTCTTCCTCCTTCG | |
| | GTGCGTCGCTTCCTGCATTGTTGAGCTCATCGTCAGTGTTATATATTCTTTC | |
| | AACATCTGCTTTAATGAGATATGCGAGACGCCTATGATCGCATGATATTTG | |
| | CTTTCAATTCTGTTGTGCACGTTGTAAAAAACCTGAGCATGTGTAGCTCAG | |
| | ATCCTTACCGCCGGTTTCGGTTCATTCTAATGAATATATCACCCGTTACTAT | |
| | CGTATTTTTATGAATAATATTCTCCGTTCAATTTACTGATTGTACCCTACTA | |
| | CTTATATGTACAATATTAAAATGAAAACAATATATTGTGCTGAATAGGTTT | |
| | ATAGCGACATCTATGATAGAGCGCCACAATAACAAACAATTGCGTTTTATT | |
| | ATTACAAATCCAATTTTAAAAAAAGCGGCAGAACCGGTCAAACCTAAAAG | |
| | ACTGATTACATAAATCTTATTCAAATTTCAAAAGGCCCCAGGGGCTAGTAT | |
| | CTACGACACACCGAGCGGCGAACTAATAACGTTCACTGAAGGGAACTCCG | |
| | GTTCCCCGCCGGCGCGCATGGGTGAGATTCCTTGAAGTTGAGTATTGGCCG | |
| | TCCGCTCTACCGAAAGTTACGGGCACCATTCAACCCGGTCCAGCACGGCGG | |
| | CCGGGTAACCGACTTGCTGCCCCGAGAATTATGCAGCATTTTTTTGGTGTAT | |
| | GTGGGCCCCAAATGAAGTGCAGGTCAAACCTTGACAGTGACGACAAATCG | |
| | TTGGGCGGGTCCAGGGCGAATTTTGCGACAACATGTCGAGGCTCAGCAGG | |
| | ACCTGCAGGCATGCAAGCTAGCTTACTAGTGATGCATATTCTATAGTGTCA | |
| | CCTAAATGGCGGCCGCACTAGTGATATCCCGCGGCCATGGCGGCCGGGAG | |
| | CATGCGACGTCGGGCCCAATTCGCCCTATAGTGAGTCGTATTACAATTCAC | |
| | TGGCCGTCGTTTTACAACGTCGTGACTGGGAAAACCCTGGCGTTACCCAAC | |
| | TTAATCGCCTTGCAGCACATCCCCCTTTCGCCAGCTGGCGTAATAGCGAAG | |
| | AGGCCCGCACCGATCGCCCTTCCCAACAGTTGCGCAGCCTGAATGGCGAAT | |
| | GGAAATTGTAAACGTTAATGGGTTTCTGGAGTTTAATGAGCTAAGCACATA | |
| | CGTCAGAAACCATTATTGCGCGTTCAAAAGTCGCCTAAGGTCACTATCAGC | |
| | TAGCAAATATTTCTTGTCAAAAATGCTCCACTGACGTTCCATAAATTCCCCT | |
| | CGGTATCCAATTAGAGTCTCATATTCACTCTCAATCCAAATAATCTGCAAT | |
| | GGCAATTACCTTATCCGCAACTTCTTTACCTATTTCCGCCCGGATCCGGGCA | |
| | GGTTCTCCGGCCGCTTGGGTGGAGAGGCTATTCGGCTATGACTGGGCACAA | |
| | CAGACAATCGGCTGCTCTGATGCCGCCGTGTTCCGGCTGTCAGCGCAGGGG | |
| | CGCCCGGTTCTTTTTGTCAAGACCGACCTGTCCGGTGCCCTGAATGAACTG | |
| | CAGGACGAGGCAGCGCGGCTATCGTGGCTGGCCACGACGGGCGTTCCTTG | |
| | CGCAGCTGTGCTCGACGTTGTCACTGAAGCGGGAAGGGACTGGCTGCTATT | |
| | GGGCGAAGTGCCGGGGCAGGATCTCCTGTCATCTCACCTTGCTCCTGCCGA | |
| | GAAAGTATCCATCATGGCTGATGCAATGCGGCGGCTGCATACGCTTGATCC | |
| | GGCTACCTGCCCATTCGACCACCAAGCGAAACATCGCATCGAGCGAGCAC | |
| | GTACTCGGATGGAAGCCGGTCTTGTCGATCAGGATGATCTGGACGAAGAG | |
| | CATCAGGGGCTCGCGCCAGCCGAACTGTTCGCCAGGCTCAAGGCGCGCAT | |
| | GCCCGACGGCGAGGATCTCGTCGTGACCCATGGCGATGCCTGCTTGCCGAA | |
| | TATCATGGTGGAAAATGGCCGCTTTTCTGGATTCATCGACTGTGGCCGGCT | |
| | GGGTGTGGCGGACCGCTATCAGGACATAGCGTTGGCTACCCGTGATATTGC | |
| | TGAAGAGCTTGGCGGCGAATGGGCTGACCGCTTCCTCGTGCTTTACGGTAT | |
| | CGCCGCTCCCGATTCGCAGCGCATCGCCTTCTATCGCCTTCTTGACGAGTTC | |
| | TTCTGAGCGGGACTCTGGGGTTCGAAATGACCGACCAAGCGACGCCCAAC | |
| | CTGCCATCACGAGATTTCGATTCCACCGCCGCCTTCTATGAAAGGTTGGGC | |
| | TTCGGAATCGTTTTCCGGGACGCCGGCTGGATGATCCTCCAGCGCGGGGAT | |
| | CTCATGCTGGAGTTCTTCGCCCACCCCGATCCAACACTTACGTTTGCAACGT | |
| | CCAAGAGCAAATAGACCACGAACGCCGGAAGGTTGCCGCAGCGTGTGGAT | |
| | TGCGTCTCAATTCTCTCTTGCAGGAATGCAATGATGAATATGATACTGACT | |
| | ATGAAACTTTGAGGGAATACTGCCTAGCACCGTCACCTCATAACGTGCATC | |
| | ATGCATGCCCTGACAACATGGAACATCGCTATTTTTCTGAAGAATTATGCT | |
| | CGTTGGAGGATGTCGCGGCAATTGCAGCTATTGCCAACATCGAACTACCCC | |
| | TCACGCATGCATTCATCAATATTATTCATGCGGGGAAAGGCAAGATTAATC | |
| | CAACTGGCAAATCATCCAGCGTGATTGGTAACTTCAGTTCCAGCGACTTGA | |
| | TTCGTTTTGGTGCTACCCACGTTTTCAATAAGGACGAGATGGTGGAGTAAA | |
| | GAAGGAGTGCGTCGAAGCAGATCGTTCAAACATTTGGCAATAAAGTTTCTT | |
| | AAGATTGAATCCTGTTGCCGGTCTTGCGATGATTATCATATAATTTCTGTTG | |
| | AATTACGTTAAGCATGTAATAATTAACATGTAATGCATGACGTTATTTATG | |
| | AGATGGGTTTTTATGATTAGAGTCCCGCAATTATACATTTAATACGCGATA | |
| | GAAAACAAAATATAGCGCGCAAACTAGGATAAATTATCGCGCGCGGTGTC | |
| | ATCTATGTTACTAGATCGAATTAATTCCAGGCGGTGAAGGGCAATCAGCTG | |
| | TTGCCCGTCTCACTGGTGAAAAGAAAAACCACCCCAGTACATTAAAAACGT | |
| | CCGCAATGTGTTATTAAGTTGTCTAAGCGTCAATTTGTTTACACCACAATAT | |
| | ATCCTGCCACCAGCCAGCCAACAGCTCCCCGACCGGCAGCTCGGCACAAA | |
| | ATCACCACTCGATACAGGCAGCCCATCAGTCCGGGACGGCGTCAGCGGGA | |
| | GAGCCGTTGTAAGGCGGCAGACTTTGCTCATGTTACCGATGCTATTCGGAA | |
| | GAACGGCAACTAAGCTGCCGGGTTTGAAACACGGATGATCTCGCGGAGGG | |
| | TAGCATGTTGATTGTAACGATGACAGAGCGTTGCTGCCTGTGATCAAATAT | |
| | CATCTCCCTCGCAGAGATCCGAATTATCAGCCTTCTTATTCATTTCTCGCTT | |
| | AACCGTGACAGGCTGTCGATCTTGAGAACTATGCCGACATAATAGGAAATC | |
| | GCTGGATAAAGCCGCTGAGGAAGCTGAGTGGCGCTATTTCTTTAGAAGTGA | |
| | ACGTTGACGATGTCGACGGATCTTTTCCGCTGCATAACCCTGCTTCGGGGT | |
| | CATTATAGCGATTTTTTCGGTATATCCATCCTTTTTCGCACGATATACAGGA | |
| | TTTTGCCAAAGGGTTCGTGTAGACTTTCCTTGGTGTATCCAACGGCGTCAGC | |
| | CGGGCAGGATAGGTGAAGTAGGCCCACCCGCGAGCGGGTGTTCCTTCTTCA | |
| | CTGTCCCTTATTCGCACCTGGCGGTGCTCAACGGGAATCCTGCTCTGCGAG | |
| | GCTGGCCGGCTACCGCCGGCGTAACAGATGAGGGCAAGCGGATGGCTGAT | |
| | GAAACCAAGCCAACCAGGGGTGATGCTGCCAACTTACTGATTTAGTGTATG | |
| | ATGGTGTTTTTGAGGTGCTCCAGTGGCTTCTGTTTCTATCAGCTGTCCCTCC | |
| | TGTTCAGCTACTGACGGGGTGGTGCGTAACGGCAAAAGCACCGCCGGACA | |
| | TCAGCGCTATCTCTGCTCTCACTGCCGTAAAACATGGCAACTGCAGTTCAC | |
| | TTACACCGCTTCTCAACCCGGTACGCACCAGAAAATCATTGATATGGCCAT | |
| | GAATGGCGTTGGATGCCGGGCAACAGCCCGCATTATGGGCGTTGGCCTCAA | |
| | CACGATTTTACGTCACTTAAAAAACTCAGGCCGCAGTCGGTAACCTCGCGC | |
| | ATACAGCCGGGCAGTGACGTCATCGTCTGCGCGGAAATGGACGAACAGTG | |
| | GGGCTATGTCGGGGCTAAATCGCGCCAGCGCTGGCTGTTTTACGCGTATGA | |
| | CAGTCTCCGGAAGACGGTTGTTGCGCACGTATTCGGTGAACGCACTATGGC | |
| | GACGCTGGGGCGTCTTATGAGCCTGCTGTCACCCTTTGACGTGGTGATATG | |
| | GATGACGGATGGCTGGCCGCTGTATGAATCCCGCCTGAAGGGAAAGCTGC | |
| | ACGTAATCAGCAAGCGATATACGCAGCGAATTGAGCGGCATAACCTGAAT | |
| | CTGAGGCAGCACCTGGCACGGCTGGGACGGAAGTCGCTGTCGTTCTCAAA | |
| | ATCGGTGGAGCTGCATGACAAAGTCATCGGGCATTATCTGAACATAAAAC | |
| | ACTATCAATAAGTTGGAGTCATTACCCAACCAGGAAGGGCAGCCCACCTAT | |
| | CAAGGTGTACTGCCTTCCAGACGAACGAAGAGCGATTGAGGAAAAGGCGG | |
| | CGGCGGCCGGCATGAGCCTGTCGGCCTACCTGCTGGCCGTCGGCCAGGGCT | |
| | ACAAAATCACGGGCGTCGTGGACTATGAGCACGTCCGCGAGCTGGCCCGC | |
| | ATCAATGGCGACCTGGGCCGCCTGGGCGGCCTGCTGAAACTCTGGCTCACC | |
| | GACGACCCGCGCACGGCGCGGTTCGGTGATGCCACGATCCTCGCCCTGCTG | |
| | GCGAAGATCGAAGAGAAGCAGGACGAGCTTGGCAAGGTCATGATGGGCGT | |
| | GGTCCGCCCGAGGGCAGAGCCATGACTTTTTTAGCCGCTAAAACGGCCGGG | |
| | GGGTGCGCGTGATTGCCAAGCACGTCCCCATGCGCTCCATCAAGAAGAGC | |
| | GACTTCGCGGAGCTGGTATTCGTGCAGGGCAAGATTCGGAATACCAAGTAC | |
| | GAGAAGGACGGCCAGACGGTCTACGGGACCGACTTCATTGCCGATAAGGT | |
| | GGATTATCTGGACACCAAGGCACCAGGCGGGTCAAATCAGGAATAAGGGC | |
| | ACATTGCCCCGGCGTGAGTCGGGGCAATCCCGCAAGGAGGGTGAATGAAT | |
| | CGGACGTTTGACCGGAAGGCATACAGGCAAGAACTGATCGACGCGGGGTT | |
| | TTCCGCCGAGGATGCCGAAACCATCGCAAGCCGCACCGTCATGCGTGCGCC | |
| | CCGCGAAACCTTCCAGTCCGTCGGCTCGATGGTCCAGCAAGCTACGGCCAA | |
| | GATCGAGCGCGACAGCGTGCAACTGGCTCCCCCTGCCCTGCCCGCGCCATC | |
| | GGCCGCCGTGGAGCGTTCGCGTCGTCTCGAACAGGAGGCGGCAGGTTTGG | |
| | CGAAGTCGATGACCATCGACACGCGAGGAACTATGACGACCAAGAAGCGA | |
| | AAAACCGCCGGCGAGGACCTGGCAAAACAGGTCAGCGAGGCCAAGCAGG | |
| | CCGCGTTGCTGAAACACACGAAGCAGCAGATCAAGGAAATGCAGCTTTCC | |
| | TTGTTCGATATTGCGCCGTGGCCGGACACGATGCGAGCGATGCCAAACGAC | |
| | ACGGCCCGCTCTGCCCTGTTCACCACGCGCAACAAGAAAATCCCGCGCGAG | |
| | GCGCTGCAAAACAAGGTCATTTTCCACGTCAACAAGGACGTGAAGATCAC | |
| | CTACACCGGCGTCGAGCTGCGGGCCGACGATGACGAACTGGTGTGGCAGC | |
| | AGGTGTTGGAGTACGCGAAGCGCACCCCTATCGGCGAGCCGATCACCTTCA | |
| | CGTTCTACGAGCTTTGCCAGGACCTGGGCTGGTCGATCAATGGCCGGTATT | |
| | ACACGAAGGCCGAGGAATGCCTGTCGCGCCTACAGGCGACGGCGATGGGC | |
| | TTCACGTCCGACCGCGTTGGGCACCTGGAATCGGTGTCGCTGCTGCACCGC | |
| | TTCCGCGTCCTGGACCGTGGCAAGAAAACGTCCCGTTGCCAGGTCCTGATC | |
| | GACGAGGAAATCGTCGTGCTGTTTGCTGGCGACCACTACACGAAATTCATA | |
| | TGGGAGAAGTACCGCAAGCTGTCGCCGACGGCCCGACGGATGTTCGACTA | |
| | TTTCAGCTCGCACCGGGAGCCGTACCCGCTCAAGCTGGAAACCTTCCGCCT | |
| | CATGTGCGGATCGGATTCCACCCGCGTGAAGAAGTGGCGCGAGCAGGTCG | |
| | GCGAAGCCTGCGAAGAGTTGCGAGGCAGCGGCCTGGTGGAACACGCCTGG | |
| | GTCAATGATGACCTGGTGCATTGCAAACGCTAGGGCCTTGTGGGGTCAGTT | |
| | CCGGCTGGGGGTTCAGCAGCCAGCGCTTTACTGGCATTTCAGGAACAAGCG | |
| | GGCACTGCTCGACGCACTTGCTTCGCTCAGTATCGCTCGGGACGCACGGCG | |
| | CGCTCTACGAACTGCCGATAAACAGAGGATTAAAATTGACAATTGTGATTA | |
| | AGGCTCAGATTCGACGGCTTGGAGCGGCCGACGTGCAGGATTTCCGCGAG | |
| | ATCCGATTGTCGGCCCTGAAGAAAGCTCCAGAGATGTTCGGGTCCGTTTAC | |
| | GAGCACGAGGAGAAAAAGCCCATGGAGGCGTTCGCTGAACGGTTGCGAGA | |
| | TGCCGTGGCATTCGGCGCCTACATCGACGGCGAGATCATTGGGCTGTCGGT | |
| | CTTCAAACAGGAGGACGGCCCCAAGGACGCTCACAAGGCGCATCTGTCCG | |
| | GCGTTTTCGTGGAGCCCGAACAGCGAGGCCGAGGGGTCGCCGGTATGCTG | |
| | CTGCGGGCGTTGCCGGCGGGTTTATTGCTCGTGATGATCGTCCGACAGATT | |
| | CCAACGGGAATCTGGTGGATGCGCATCTTCATCCTCGGCGCACTTAATATT | |
| | TCGCTATTCTGGAGCTTGTTGTTTATTTCGGTCTACCGCCTGCCGGGCGGGG | |
| | TCGCGGCGACGGTAGGCGCTGTGCAGCCGCTGATGGTCGTGTTCATCTCTG | |
| | CCGCTCTGCTAGGTAGCCCGATACGATTGATGGCGGTCCTGGGGGCTATTT | |
| | GCGGAACTGCGGGCGTGGCGCTGTTGGTGTTGACACCAAACGCAGCGCTA | |
| | GATCCTGTCGGCGTCGCAGCGGGCCTGGCGGGGGCGGTTTCCATGGCGTTC | |
| | GGAACCGTGCTGACCCGCAAGTGGCAACCTCCCGTGCCTCTGCTCACCTTT | |
| | ACCGCCTGGCAACTGGCGGCCGGAGGACTTCTGCTCGTTCCAGTAGCTTTA | |
| | GTGTTTGATCCGCCAATCCCGATGCCTACAGGAACCAATGTTCTCGGCCTG | |
| | GCGTGGCTCGGCCTGATCGGAGCGGGTTTAACCTACTTCCTTTGGTTCCGG | |
| | GGGATCTCGCGACTCGAACCTACAGTTGTTTCCTTACTGGGCTTTCTCAGCC | |
| | GGGATGGCGCTAAGAAGCTATTGCCGCCGATCTTCATATGCGGTGTGAAAT | |
| | ACCGCACAGATGCGTAAGGAGAAAATACCGCATCAGGCGCTCTTCCGCTTC | |
| | CTCGCTCACTGACTCGCTGCGCTCGGTCGTTCGGCTGCGGCGAGCGGTATC | |
| | AGCTCACTCAAAGGCGGTAATACGGTTATCCACAGAATCAGGGGATAACG | |
| | CAGGAAAGAACATGTGAGCAAAAGGCCAGCAAAAGGCCAGGAACCGTAA | |
| | AAAGGCCGCGTTGCTGGCGTTTTTCCATAGGCTCCGCCCCCCTGACGAGCA | |
| | TCACAAAAATCGACGCTCAAGTCAGAGGTGGCGAAACCCGACAGGACTAT | |
| | AAAGATACCAGGCGTTTCCCCCTGGAAGCTCCCTCGTGCGCTCTCCTGTTCC | |
| | GACCCTGCCGCTTACCGGATACCTGTCCGCCTTTCTCCCTTCGGGAAGCGTG | |
| | GCGCTTTCTCAATGCTCACGCTGTAGGTATCTCAGTTCGGTGTAGGTCGTTC | |
| | GCTCCAAGCTGGGCTGTGTGCACGAACCCCCCGTTCAGCCCGACCGCTGCG | |
| | CCTTATCCGGTAACTATCGTCTTGAGTCCAACCCGGTAAGACACGACTTAT | |
| | CGCCACTGGCAGCAGCCACTGGTAACAGGATTAGCAGAGCGAGGTATGTA | |
| | GGCGGTGCTACAGAGTTCTTGAAGTGGTGGCCTAACTACGGCTACACTAGA | |
| | AGGACAGTATTTGGTATCTGCGCTCTGCTGAAGCCAGTTACCTTCGGAAAA | |
| | AGAGTTGGTAGCTCTTGATCCGGCAAACAAACCACCGCTGGTAGCGGTGGT | |
| | TTTTTTGTTTGCAAGCAGCAGATTACGCGCAGAAAAAAAGGATATCAAGAA | |
| | GATCCTTTGATCTTTTCTACGGGGTCTGACGCTCAGTGGAACGAAAACTCA | |
| | CGTTAAGGGATTTTGGTCATGAGATTATCAAAAAGGATCTTCACCTAGATC | |
| | CTTTTAAATTAAAAATGAAGTTTTAAATCAATCTAAAGTATATATGAGTAA | |
| | ACTTGGTCTGACAGTTACCAATGCTTAATCAGTGAGGCACCTATCTCAGCG | |
| | ATCTGTCTATTTCGTTCATCCATAGTTGCCTGACTCCCCGTCGTGTAGATAA | |
| | CTACGATACGGGAGGGCTTACCATCTGGCCCCAGTGCTGCAATGATACCGC | |
| | GAGACCCACGCTCACCGGCTCCAGATTTATCAGCAATAAACCAGCCAGCCG | |
| | GAAGGGCCGAGCGCAGAAGTGGTCCTGCAACTTTATCCGCCTCCATCCAGT | |
| | CTATTAAACAAGTGGCAGCAACGGATTCGCAAACCTGTCACGCCTTTTGTG | |
| | CCAAAAGCCGCGCCAGGTTTGCGATCCGCTGTGCCAGGCGTTAGGCGTCAT | |
| | ATGAAGATTTCGGTGATCCCTGAGCAGGTGGCGGAAACATTGGATGCTGA | |
| | GAACCATTTCATTGTTCGTGAAGTGTTCGATGTGCACCTATCCGACCAAGG | |
| | CTTTGAACTATCTACCAGAAGTGTGAGCCCCTACCGGAAGGATTACATCTC | |
| | GGATGATGACTCTGATGAAGACTCTGCTTGCTATGGCGCATTCATCGACCA | |
| | AGAGCTTGTCGGGAAGATTGAACTCAACTCAACATGGAACGATCTAGCCTC | |
| | TATCGAACACATTGTTGTGTCGCACACGCACCGAGGCAAAGGAGTCGCGC | |
| | ACAGTCTCATCGAATTTGCGAAAAAGTGGGCACTAAGCAGACAGCTCCTTG | |
| | GCATACGATTAGAGACACAAACGAACAATGTACCTGCCTGCAATTTGTACG | |
| | CAAAATGTGGCTTTACTCTCGGCGGCATTGACCTGTTCACGTATAAAACTA | |
| | GACCTCAAGTCTCGAACGAAACAGCGATGTACTGGTACTGGTTCTCGGGAG | |
| | CACAGGATGACGCCTAACAATTCATTCAAGCCGACACCGCTTCGCGGCGCG | |
| | GCTTAATTCAGGAGTTAAACATCATGAGGGAAGCGGTGATCGCCGAAGTA | |
| | TCGACTCAACTATCAGAGGTAGTTGGCGTCATCGAGCGCCATCTCGAACCG | |
| | ACGTTGCTGGCCGTACATTTGTACGGCTCCGCAGTGGATGGCGGCCTGAAG | |
| | CCACACAGTGATATTGATTTGCTGGTTACGGTGACCGTAAGGCTTGATGAA | |
| | ACAACGCGGCGAGCTTTGATCAACGACCTTTTGGAAACTTCGGCTTCCCCT | |
| | GGAGAGAGCGAGATTCTCCGCGCTGTAGAAGTCACCATTGTTGTGCACGAC | |
| | GACATCATTCCGTGGCGTTATCCAGCTAAGCGCGAACTGCAATTTGGAGAA | |
| | TGGCAGCGCAATGACATTCTTGCAGGTATCTTCGAGCCAGCCACGATCGAC | |
| | ATTGATCTGGCTATCTTGCTGACAAAAGCAAGAGAACATAGCGTTGCCTTG | |
| | GTAGGTCCAGCGGCGGAGGAACTCTTTGATCCGGTTCCTGAACAGGATCTA | |
| | TTTGAGGCGCTAAATGAAACCTTAACGCTATGGAACTCGCCGCCCGACTGG | |
| | GCTGGCGATGAGCGAAATGTAGTGCTTACGTTGTCCCGCATTTGGTACAGC | |
| | GCAGTAACCGGCAAAATCGCGCCGAAGGATGTCGCTGCCGACTGGGCAAT | |
| | GGAGCGCCTGCCGGCCCAGTATCAGCCCGTCATACTTGAAGCTAGGCAGGC | |
| | TTATCTTGGACAAGAAGATCGCTTGGCCTCGCGCGCAGATCAGTTGGAAGA | |
| | ATTTGTTCACTACGTGAAAGGCGAGATCACCAAGGTAGTCGGCAAATAATG | |
| | TCTAACAATTCGTTCAAGCCGACGCCGCTTCGCGGCGCGGCTTAACTCAAG | |
| | CGTTAGAGAGCTGGGGAAGACTATGCGCGATCTGTTGAAGGTGGTTCTAAG | |
| | CCTCGTACTTGCGATGGCATCGGGGCAGGCACTTGCTGACCTGCCAATTGT | |
| | TTTAGTGGATGAAGCTCGTCTTCCCTATGACTACTCCCCATCCAACTACGAC | |
| | ATTTCTCCAAGCAACTACGACAACTCCATAAGCAATTACGACAATAGTCCA | |
| | TCAAATTACGACAACTCTGAGAGCAACTACGATAATAGTTCATCCAATTAC | |
| | GACAATAGTCGCAACGGAAATCGTAGGCTTATATATAGCGCAAATGGGTCT | |
| | CGCACTTTCGCCGGCTACTACGTCATTGCCAACAATGGGACAACGAACTTC | |
| | TTTTCCACATCTGGCAAAAGGATGTTCTACACCCCAAAAGGGGGGCGCGGC | |
| | GTCTATGGCGGCAAAGATGGGAGCTTCTGCGGGGCATTGGTCGTCATAAAT | |
| | GGCCAATTTTCGCTTGCCCTGACAGATAACGGCCTGAAGATCATGTATCTA | |
| | AGCAACTAGCCTGCTCTCTAATAAAATGTTAGGAGCTTGGCTGCCATTTTT | |
| | GGGGTGAGGCCGTTCGCGGCCGAGGGGCGCAGCCCCTGGGGGGATGGGAG | |
| | GCCCGCGTTAGCGGGCCGGGAGGGTTCGAGAAGGGGGGGCACCCCCCTTC | |
| | GGCGTGCGCGGTCACGCGCCAGGGCGCAGCCCTGGTTAAAAACAAGGTTT | |
| | ATAAATATTGGTTTAAAAGCAGGTTAAAAGACAGGTTAGCGGTGGCCGAA | |
| | AAACGGGCGGAAACCCTTGCAAATGCTGGATTTTCTGCCTGTGGACAGCCC | |
| | CTCAAATGTCAATAGGTGCGCCCCTCATCTGTCAGCACTCTGCCCCTCAAG | |
| | TGTCAAGGATCGCGCCCCTCATCTGTCAGTAGTCGCGCCCCTCAAGTGTCA | |
| | ATACCGCAGGGCACTTATCCCCAGGCTTGTCCACATCATCTGTGGGAAACT | |
| | CGCGTAAAATCAGGCGTTTTCGCCGATTTGCGAGGCTGGCCAGCTCCACGT | |
| | CGCCGGCCGAAATCGAGCCTGCCCCTCATCTGTCAACGCCGCGCCGGGTGA | |
| | GTCGGCCCCTCAAGTGTCAACGTCCGCCCCTCATCTGTCAGTGAGGGCCAA | |
| | GTTTTCCGCGAGGTATCCACAACGCCGGCGGCCGGCCGCGGTGTCTCGCAC | |
| | ACGGCTTCGACGGCGTTTCTGGCGCGTTTGCAGGGCCATAGACGGCCGCCA | |
| | GCCCAGCGGCGAGGGCAACCAGCCCGGTGAGCGTCGGAAAGGG | |
| V2 | >pART27_hpV2_sequence | 2 |
| | TCGACATCTTGCTGCGTTCGGATATTTTCGTGGAGTTCCCGCCACAGACCCG | |
| | GATTGAAGGCGAGATCCAGCAACTCGCGCCAGATCATCCTGTGACGGAAC | |
| | TTTGGCGCGTGATGACTGGCCAGGACGTCGGCCGAAAGAGCGACAAGCAG | |
| | ATCACGATTTTCGACAGCGTCGGATTTGCGATCGAGGATTTTTCGGCGCTG | |
| | CGCTACGTCCGCGACCGCGTTGAGGGATCAAGCCACAGCAGCCCACTCGA | |
| | CCTTCTAGCCGACCCAGACGAGCCAAGGGATCTTTTTGGAATGCTGCTCCG | |
| | TCGTCAGGCTTTCCGACGTTTGGGTGGTTGAACAGAAGTCATTATCGTACG | |
| | GAATGCCAGCACTCCCGAGGGGAACCCTGTGGTTGGCATGCACATACAAA | |
| | TGGACGAACGGATAAACCTTTTCACGCCCTTTTAAATATCCGTTATTCTAAT | |
| | AAACGCTCTTTTCTCTTAGGTTTACCCGCCAATATATCCTGTCAAACACTGA | |
| | TAGTTTAAACTGAAGGCGGGAAACGACAATCTGATCATGAGCGGAGAATT | |
| | AAGGGAGTCACGTTATGACCCCCGCCGATGACGCGGGACAAGCCGTTTTAC | |
| | GTTTGGAACTGACAGAACCGCAACGATTGAAGGAGCCACTCAGCCCCAAT | |
| | ACGCAAACCGCCTCTCCCCGCGCGTTGGCCGATTCATTAATGCAGCTGGCA | |
| | CGACAGGTTTCCCGACTGGAAAGCGGGCAGTGAGCGCAACGCAATTAATG | |
| | TGAGTTAGCTCACTCATTAGGCACCCCAGGCTTTACACTTTATGCTTCCGGC | |
| | TCGTATGTTGTGTGGAATTGTGAGCGGATAACAATTTCACACAGGAAACAG | |
| | CTATGACCATGATTACGCCAAGCTATTTAGGTGACACTATAGAATACTCAA | |
| | GCTATGCATCCAACGCGTTGGGAGCTCTCCCATATCGACCTGCAGGCGGCC | |
| | GCTCGACGAATTAATTCCAATCCCACAAAAATCTGAGCTTAACAGCACAGT | |
| | TGCTCCTCTCAGAGCAGAATCGGGTATTCAACACCCTCATATCAACTACTA | |
| | CGTTGTGTATAACGGTCCACATGCCGGTATATACGATGACTGGGGTTGTAC | |
| | AAAGGCGGCAACAAACGGCGTTCCCGGAGTTGCACACAAGAAATTTGCCA | |
| | CTATTACAGAGGCAAGAGCAGCAGCTGACGCGTACACAACAAGTCAGCAA | |
| | ACAGACAGGTTGAACTTCATCCCCAAAGGAGAAGCTCAACTCAAGCCCAA | |
| | GAGCTTTGCTAAGGCCCTAACAAGCCCACCAAAGCAAAAAGCCCACTGGC | |
| | TCACGCTAGGAACCAAAAGGCCCAGCAGTGATCCAGCCCCAAAAGAGATC | |
| | TCCTTTGCCCCGGAGATTACAATGGACGATTTCCTCTATCTTTACGATCTAG | |
| | GAAGGAAGTTCGAAGGTGAAGGTGACGACACTATGTTCACCACTGATAAT | |
| | GAGAAGGTTAGCCTCTTCAATTTCAGAAAGAATGCTGACCCACAGATGGTT | |
| | AGAGAGGCCTACGCAGCAGGTCTCATCAAGACGATCTACCCGAGTAACAA | |
| | TCTCCAGGAGATCAAATACCTTCCCAAGAAGGTTAAAGATGCAGTCAAAA | |
| | GATTCAGGACTAATTGCATCAAGAACACAGAGAAAGACATATTTCTCAAG | |
| | ATCAGAAGTACTATTCCAGTATGGACGATTCAAGGCTTGCTTCATAAACCA | |
| | AGGCAAGTAATAGAGATTGGAGTCTCTAAAAAGGTAGTTCCTACTGAATCT | |
| | AAGGCCATGCATGGAGTCTAAGATTCAAATCGAGGATCTAACAGAACTCG | |
| | CCGTGAAGACTGGCGAACAGTTCATACAGAGTCTTTTACGACTCAATGACA | |
| | AGAAGAAAATCTTCGTCAACATGGTGGAGCACGACACTCTGGTCTACTCCA | |
| | AAAATGTCAAAGATACAGTCTCAGAAGACCAAAGGGCTATTGAGACTTTTC | |
| | AACAAAGGATAATTTCGGGAAACCTCCTCGGATTCCATTGCCCAGCTATCT | |
| | GTCACTTCATCGAAAGGACAGTAGAAAAGGAAGGTGGCTCCTACAAATGC | |
| | CATCATTGCGATAAAGGAAAGGCTATCATTCAAGATCTCTCTGCCGACAGT | |
| | GGTCCCAAAGATGGACCCCCACCCACGAGGAGCATCGTGGAAAAAGAAGA | |
| | CGTTCCAACCACGTCTTCAAAGCAAGTGGATTGATGTGACATCTCCACTGA | |
| | CGTAAGGGATGACGCACAATCCCACTATCCTTCGCAAGACCCTTCCTCTAT | |
| | ATAAGGAAGTTCATTTCATTTGGAGAGGACACGATGGTAACACTGAACAA | |
| | ACGGAATCGCGTTCTTCCTGAGTGCGATTCCTGCAGTTCTAGTGAAAGTTCT | |
| | TTGAATGATATTGATATTTGTGGTGATGATGATGGGTTAGGGGATGAGGCT | |
| | TTAGACGCTGGATCCGTTTATTCGTCGTCACAGAAACTGTTAGTTTCTGTGG | |
| | CTAAAGATGTTCTTTTAGATGACTGTGATTCAACGATATTGGATATATCGTT | |
| | GCCTTCTGCTTTATGGTTTTTGTCGCAAAGATATTTGACTTGTTGTTTGAGG | |
| | AAAGAATTACTGCCTCTGCCAGGTATATCCGAGAAACAGACTGTTTTATTG | |
| | CGACAGCTGATTAGGCGTGTCGCTCGTCGTCATTGTTTATTTACTTACAAGT | |
| | GCGAGGAGTGGTTTGAGGGTTGTTTGAAGATAAAGAAGGATGGTAATGAA | |
| | AAAAAGGAGCCGCCAACGGAAGCAGAGAAGAAGGCGCAGGACGACTGGG | |
| | AGGAGTTCTGCCGTAAGGCGGCGTGCTCGGCCTCGTAGTCTTTTTTCCTTTT | |
| | AGTATAAAATAGTTAAGTGATGTTAATTAGTATGATTATAATAATATAGTT | |
| | GTTATAATTGTGAAAAAATAATTTATAAATATATTGTTTACATAAACAACA | |
| | TAGTAATGTAAAAAAATATGACAAGTGATGTGTAAGACGAAGAAGATAAA | |
| | AGTTGAGAGTAAGTATATTATTTTTAATGAATTTGATCGAACATGTAAGAT | |
| | GATATACTAGCATTAATATTTGTTTTAATCATAATAGTAATTCTAGCTGGTT | |
| | TGATGAATTAAATATCAATGATAAAATACTATAGTAAAAATAAGAATAAA | |
| | TAAATTAAAATAATATTTTTTTATGATTAATAGTTTATTATATAATTAAATA | |
| | TCTATACCATTACTAAATATTTTAGTTTAAAAGTTAATAAATATTTTGTTAG | |
| | AAATTCCAATCTGCTTGTAATTTATCAATAAACAAAATATTAAATAACAAG | |
| | CTAAAGTAACAAATAATATCAAACTAATAGAAACAGTAATCTAATGTAAC | |
| | AAAACATAATCTAATGCTAATATAACAAAGCGCAAGATCTATCATTTTATA | |
| | TAGTATTATTTTCAATCAACATTCTTATTAATTTCTAAATAATACTTGTAGT | |
| | TTTATTAACTTCTAAATGGATTGACTATTAATTAAATGAATTAGTCGAACAT | |
| | GAATAAACAAGGTAACATGATAGATCATGTCATTGTGTTATCATTGATCTT | |
| | ACATTTGGATTGCTACGAGGCCGAGCACGCCGCCTTACGGCAGAACTCCTC | |
| | CCAGTCGTCCTGCGCCTTCTTCTCTGCTTCCGTTGGCGGCTCCTTTTTTTCAT | |
| | TACCATCCTTCTTTATCTTCAAACAACCCTCAAACCACTCCTCGCACTTGTA | |
| | AGTAAATAAACAATGACGACGAGCGACACGCCTAATCAGCTGTCGCAATA | |
| | AAACAGTCTGTTTCTCGGATATACCTGGCAGAGGCAGTAATTCTTTCCTCA | |
| | AACAACAAGTCAAATATCTTTGCGACAAAAACCATAAAGCAGAAGGCAAC | |
| | GATATATCCAATATCGTTGAATCACAGTCATCTAAAAGAACATCTTTAGCC | |
| | ACAGAAACTAACAGTTTCTGTGACGACGAATAAACGGATCCAGCGTCTAA | |
| | AGCCTCATCCCCTAACCCATCATCATCACCACAAATATCAATATCATTCAA | |
| | AGAACTTTCACTAGAACTGCAGGAATCGCACTCAGGAAGAACGCGATTCC | |
| | GTTTGTTCAGTGTTACCATCTGCTTTAATGAGATATGCGAGACGCCTATGAT | |
| | CGCATGATATTTGCTTTCAATTCTGTTGTGCACGTTGTAAAAAACCTGAGCA | |
| | TGTGTAGCTCAGATCCTTACCGCCGGTTTCGGTTCATTCTAATGAATATATC | |
| | ACCCGTTACTATCGTATTTTTATGAATAATATTCTCCGTTCAATTTACTGAT | |
| | TGTACCCTACTACTTATATGTACAATATTAAAATGAAAACAATATATTGTG | |
| | CTGAATAGGTTTATAGCGACATCTATGATAGAGCGCCACAATAACAAACA | |
| | ATTGCGTTTTATTATTACAAATCCAATTTTAAAAAAAGCGGCAGAACCGGT | |
| | CAAACCTAAAAGACTGATTACATAAATCTTATTCAAATTTCAAAAGGCCCC | |
| | AGGGGCTAGTATCTACGACACACCGAGCGGCGAACTAATAACGTTCACTG | |
| | AAGGGAACTCCGGTTCCCCGCCGGCGCGCATGGGTGAGATTCCTTGAAGTT | |
| | GAGTATTGGCCGTCCGCTCTACCGAAAGTTACGGGCACCATTCAACCCGGT | |
| | CCAGCACGGCGGCCGGGTAACCGACTTGCTGCCCCGAGAATTATGCAGCAT | |
| | TTTTTTGGTGTATGTGGGCCCCAAATGAAGTGCAGGTCAAACCTTGACAGT | |
| | GACGACAAATCGTTGGGCGGGTCCAGGGCGAATTTTGCGACAACATGTCG | |
| | AGGCTCAGCAGGACCTGCAGGCATGCAAGCTAGCTTACTAGTGATGCATAT | |
| | TCTATAGTGTCACCTAAATGGCGGCCGCACTAGTGATATCCCGCGGCCATG | |
| | GCGGCCGGGAGCATGCGACGTCGGGCCCAATTCGCCCTATAGTGAGTCGTA | |
| | TTACAATTCACTGGCCGTCGTTTTACAACGTCGTGACTGGGAAAACCCTGG | |
| | CGTTACCCAACTTAATCGCCTTGCAGCACATCCCCCTTTCGCCAGCTGGCGT | |
| | AATAGCGAAGAGGCCCGCACCGATCGCCCTTCCCAACAGTTGCGCAGCCTG | |
| | AATGGCGAATGGAAATTGTAAACGTTAATGGGTTTCTGGAGTTTAATGAGC | |
| | TAAGCACATACGTCAGAAACCATTATTGCGCGTTCAAAAGTCGCCTAAGGT | |
| | CACTATCAGCTAGCAAATATTTCTTGTCAAAAATGCTCCACTGACGTTCCAT | |
| | AAATTCCCCTCGGTATCCAATTAGAGTCTCATATTCACTCTCAATCCAAATA | |
| | ATCTGCAATGGCAATTACCTTATCCGCAACTTCTTTACCTATTTCCGCCCGG | |
| | ATCCGGGCAGGTTCTCCGGCCGCTTGGGTGGAGAGGCTATTCGGCTATGAC | |
| | TGGGCACAACAGACAATCGGCTGCTCTGATGCCGCCGTGTTCCGGCTGTCA | |
| | GCGCAGGGGCGCCCGGTTCTTTTTGTCAAGACCGACCTGTCCGGTGCCCTG | |
| | AATGAACTGCAGGACGAGGCAGCGCGGCTATCGTGGCTGGCCACGACGGG | |
| | CGTTCCTTGCGCAGCTGTGCTCGACGTTGTCACTGAAGCGGGAAGGGACTG | |
| | GCTGCTATTGGGCGAAGTGCCGGGGCAGGATCTCCTGTCATCTCACCTTGC | |
| | TCCTGCCGAGAAAGTATCCATCATGGCTGATGCAATGCGGCGGCTGCATAC | |
| | GCTTGATCCGGCTACCTGCCCATTCGACCACCAAGCGAAACATCGCATCGA | |
| | GCGAGCACGTACTCGGATGGAAGCCGGTCTTGTCGATCAGGATGATCTGGA | |
| | CGAAGAGCATCAGGGGCTCGCGCCAGCCGAACTGTTCGCCAGGCTCAAGG | |
| | CGCGCATGCCCGACGGCGAGGATCTCGTCGTGACCCATGGCGATGCCTGCT | |
| | TGCCGAATATCATGGTGGAAAATGGCCGCTTTTCTGGATTCATCGACTGTG | |
| | GCCGGCTGGGTGTGGCGGACCGCTATCAGGACATAGCGTTGGCTACCCGTG | |
| | ATATTGCTGAAGAGCTTGGCGGCGAATGGGCTGACCGCTTCCTCGTGCTTT | |
| | ACGGTATCGCCGCTCCCGATTCGCAGCGCATCGCCTTCTATCGCCTTCTTGA | |
| | CGAGTTCTTCTGAGCGGGACTCTGGGGTTCGAAATGACCGACCAAGCGACG | |
| | CCCAACCTGCCATCACGAGATTTCGATTCCACCGCCGCCTTCTATGAAAGG | |
| | TTGGGCTTCGGAATCGTTTTCCGGGACGCCGGCTGGATGATCCTCCAGCGC | |
| | GGGGATCTCATGCTGGAGTTCTTCGCCCACCCCGATCCAACACTTACGTTT | |
| | GCAACGTCCAAGAGCAAATAGACCACGAACGCCGGAAGGTTGCCGCAGCG | |
| | TGTGGATTGCGTCTCAATTCTCTCTTGCAGGAATGCAATGATGAATATGAT | |
| | ACTGACTATGAAACTTTGAGGGAATACTGCCTAGCACCGTCACCTCATAAC | |
| | GTGCATCATGCATGCCCTGACAACATGGAACATCGCTATTTTTCTGAAGAA | |
| | TTATGCTCGTTGGAGGATGTCGCGGCAATTGCAGCTATTGCCAACATCGAA | |
| | CTACCCCTCACGCATGCATTCATCAATATTATTCATGCGGGGAAAGGCAAG | |
| | ATTAATCCAACTGGCAAATCATCCAGCGTGATTGGTAACTTCAGTTCCAGC | |
| | GACTTGATTCGTTTTGGTGCTACCCACGTTTTCAATAAGGACGAGATGGTG | |
| | GAGTAAAGAAGGAGTGCGTCGAAGCAGATCGTTCAAACATTTGGCAATAA | |
| | AGTTTCTTAAGATTGAATCCTGTTGCCGGTCTTGCGATGATTATCATATAAT | |
| | TTCTGTTGAATTACGTTAAGCATGTAATAATTAACATGTAATGCATGACGTT | |
| | ATTTATGAGATGGGTTTTTATGATTAGAGTCCCGCAATTATACATTTAATAC | |
| | GCGATAGAAAACAAAATATAGCGCGCAAACTAGGATAAATTATCGCGCGC | |
| | GGTGTCATCTATGTTACTAGATCGAATTAATTCCAGGCGGTGAAGGGCAAT | |
| | CAGCTGTTGCCCGTCTCACTGGTGAAAAGAAAAACCACCCCAGTACATTAA | |
| | AAACGTCCGCAATGTGTTATTAAGTTGTCTAAGCGTCAATTTGTTTACACCA | |
| | CAATATATCCTGCCACCAGCCAGCCAACAGCTCCCCGACCGGCAGCTCGGC | |
| | ACAAAATCACCACTCGATACAGGCAGCCCATCAGTCCGGGACGGCGTCAG | |
| | CGGGAGAGCCGTTGTAAGGCGGCAGACTTTGCTCATGTTACCGATGCTATT | |
| | CGGAAGAACGGCAACTAAGCTGCCGGGTTTGAAACACGGATGATCTCGCG | |
| | GAGGGTAGCATGTTGATTGTAACGATGACAGAGCGTTGCTGCCTGTGATCA | |
| | AATATCATCTCCCTCGCAGAGATCCGAATTATCAGCCTTCTTATTCATTTCT | |
| | CGCTTAACCGTGACAGGCTGTCGATCTTGAGAACTATGCCGACATAATAGG | |
| | AAATCGCTGGATAAAGCCGCTGAGGAAGCTGAGTGGCGCTATTTCTTTAGA | |
| | AGTGAACGTTGACGATGTCGACGGATCTTTTCCGCTGCATAACCCTGCTTC | |
| | GGGGTCATTATAGCGATTTTTTCGGTATATCCATCCTTTTTCGCACGATATA | |
| | CAGGATTTTGCCAAAGGGTTCGTGTAGACTTTCCTTGGTGTATCCAACGGC | |
| | GTCAGCCGGGCAGGATAGGTGAAGTAGGCCCACCCGCGAGCGGGTGTTCC | |
| | TTCTTCACTGTCCCTTATTCGCACCTGGCGGTGCTCAACGGGAATCCTGCTC | |
| | TGCGAGGCTGGCCGGCTACCGCCGGCGTAACAGATGAGGGCAAGCGGATG | |
| | GCTGATGAAACCAAGCCAACCAGGGGTGATGCTGCCAACTTACTGATTTAG | |
| | TGTATGATGGTGTTTTTGAGGTGCTCCAGTGGCTTCTGTTTCTATCAGCTGT | |
| | CCCTCCTGTTCAGCTACTGACGGGGTGGTGCGTAACGGCAAAAGCACCGCC | |
| | GGACATCAGCGCTATCTCTGCTCTCACTGCCGTAAAACATGGCAACTGCAG | |
| | TTCACTTACACCGCTTCTCAACCCGGTACGCACCAGAAAATCATTGATATG | |
| | GCCATGAATGGCGTTGGATGCCGGGCAACAGCCCGCATTATGGGCGTTGGC | |
| | CTCAACACGATTTTACGTCACTTAAAAAACTCAGGCCGCAGTCGGTAACCT | |
| | CGCGCATACAGCCGGGCAGTGACGTCATCGTCTGCGCGGAAATGGACGAA | |
| | CAGTGGGGCTATGTCGGGGCTAAATCGCGCCAGCGCTGGCTGTTTTACGCG | |
| | TATGACAGTCTCCGGAAGACGGTTGTTGCGCACGTATTCGGTGAACGCACT | |
| | ATGGCGACGCTGGGGCGTCTTATGAGCCTGCTGTCACCCTTTGACGTGGTG | |
| | ATATGGATGACGGATGGCTGGCCGCTGTATGAATCCCGCCTGAAGGGAAA | |
| | GCTGCACGTAATCAGCAAGCGATATACGCAGCGAATTGAGCGGCATAACC | |
| | TGAATCTGAGGCAGCACCTGGCACGGCTGGGACGGAAGTCGCTGTCGTTCT | |
| | CAAAATCGGTGGAGCTGCATGACAAAGTCATCGGGCATTATCTGAACATA | |
| | AAACACTATCAATAAGTTGGAGTCATTACCCAACCAGGAAGGGCAGCCCA | |
| | CCTATCAAGGTGTACTGCCTTCCAGACGAACGAAGAGCGATTGAGGAAAA | |
| | GGCGGCGGCGGCCGGCATGAGCCTGTCGGCCTACCTGCTGGCCGTCGGCCA | |
| | GGGCTACAAAATCACGGGCGTCGTGGACTATGAGCACGTCCGCGAGCTGG | |
| | CCCGCATCAATGGCGACCTGGGCCGCCTGGGCGGCCTGCTGAAACTCTGGC | |
| | TCACCGACGACCCGCGCACGGCGCGGTTCGGTGATGCCACGATCCTCGCCC | |
| | TGCTGGCGAAGATCGAAGAGAAGCAGGACGAGCTTGGCAAGGTCATGATG | |
| | GGCGTGGTCCGCCCGAGGGCAGAGCCATGACTTTTTTAGCCGCTAAAACGG | |
| | CCGGGGGGTGCGCGTGATTGCCAAGCACGTCCCCATGCGCTCCATCAAGAA | |
| | GAGCGACTTCGCGGAGCTGGTATTCGTGCAGGGCAAGATTCGGAATACCA | |
| | AGTACGAGAAGGACGGCCAGACGGTCTACGGGACCGACTTCATTGCCGAT | |
| | AAGGTGGATTATCTGGACACCAAGGCACCAGGCGGGTCAAATCAGGAATA | |
| | AGGGCACATTGCCCCGGCGTGAGTCGGGGCAATCCCGCAAGGAGGGTGAA | |
| | TGAATCGGACGTTTGACCGGAAGGCATACAGGCAAGAACTGATCGACGCG | |
| | GGGTTTTCCGCCGAGGATGCCGAAACCATCGCAAGCCGCACCGTCATGCGT | |
| | GCGCCCCGCGAAACCTTCCAGTCCGTCGGCTCGATGGTCCAGCAAGCTACG | |
| | GCCAAGATCGAGCGCGACAGCGTGCAACTGGCTCCCCCTGCCCTGCCCGCG | |
| | CCATCGGCCGCCGTGGAGCGTTCGCGTCGTCTCGAACAGGAGGCGGCAGG | |
| | TTTGGCGAAGTCGATGACCATCGACACGCGAGGAACTATGACGACCAAGA | |
| | AGCGAAAAACCGCCGGCGAGGACCTGGCAAAACAGGTCAGCGAGGCCAA | |
| | GCAGGCCGCGTTGCTGAAACACACGAAGCAGCAGATCAAGGAAATGCAGC | |
| | TTTCCTTGTTCGATATTGCGCCGTGGCCGGACACGATGCGAGCGATGCCAA | |
| | ACGACACGGCCCGCTCTGCCCTGTTCACCACGCGCAACAAGAAAATCCCGC | |
| | GCGAGGCGCTGCAAAACAAGGTCATTTTCCACGTCAACAAGGACGTGAAG | |
| | ATCACCTACACCGGCGTCGAGCTGCGGGCCGACGATGACGAACTGGTGTG | |
| | GCAGCAGGTGTTGGAGTACGCGAAGCGCACCCCTATCGGCGAGCCGATCA | |
| | CCTTCACGTTCTACGAGCTTTGCCAGGACCTGGGCTGGTCGATCAATGGCC | |
| | GGTATTACACGAAGGCCGAGGAATGCCTGTCGCGCCTACAGGCGACGGCG | |
| | ATGGGCTTCACGTCCGACCGCGTTGGGCACCTGGAATCGGTGTCGCTGCTG | |
| | CACCGCTTCCGCGTCCTGGACCGTGGCAAGAAAACGTCCCGTTGCCAGGTC | |
| | CTGATCGACGAGGAAATCGTCGTGCTGTTTGCTGGCGACCACTACACGAAA | |
| | TTCATATGGGAGAAGTACCGCAAGCTGTCGCCGACGGCCCGACGGATGTTC | |
| | GACTATTTCAGCTCGCACCGGGAGCCGTACCCGCTCAAGCTGGAAACCTTC | |
| | CGCCTCATGTGCGGATCGGATTCCACCCGCGTGAAGAAGTGGCGCGAGCA | |
| | GGTCGGCGAAGCCTGCGAAGAGTTGCGAGGCAGCGGCCTGGTGGAACACG | |
| | CCTGGGTCAATGATGACCTGGTGCATTGCAAACGCTAGGGCCTTGTGGGGT | |
| | CAGTTCCGGCTGGGGGTTCAGCAGCCAGCGCTTTACTGGCATTTCAGGAAC | |
| | AAGCGGGCACTGCTCGACGCACTTGCTTCGCTCAGTATCGCTCGGGACGCA | |
| | CGGCGCGCTCTACGAACTGCCGATAAACAGAGGATTAAAATTGACAATTGT | |
| | GATTAAGGCTCAGATTCGACGGCTTGGAGCGGCCGACGTGCAGGATTTCCG | |
| | CGAGATCCGATTGTCGGCCCTGAAGAAAGCTCCAGAGATGTTCGGGTCCGT | |
| | TTACGAGCACGAGGAGAAAAAGCCCATGGAGGCGTTCGCTGAACGGTTGC | |
| | GAGATGCCGTGGCATTCGGCGCCTACATCGACGGCGAGATCATTGGGCTGT | |
| | CGGTCTTCAAACAGGAGGACGGCCCCAAGGACGCTCACAAGGCGCATCTG | |
| | TCCGGCGTTTTCGTGGAGCCCGAACAGCGAGGCCGAGGGGTCGCCGGTAT | |
| | GCTGCTGCGGGCGTTGCCGGCGGGTTTATTGCTCGTGATGATCGTCCGACA | |
| | GATTCCAACGGGAATCTGGTGGATGCGCATCTTCATCCTCGGCGCACTTAA | |
| | TATTTCGCTATTCTGGAGCTTGTTGTTTATTTCGGTCTACCGCCTGCCGGGC | |
| | GGGGTCGCGGCGACGGTAGGCGCTGTGCAGCCGCTGATGGTCGTGTTCATC | |
| | TCTGCCGCTCTGCTAGGTAGCCCGATACGATTGATGGCGGTCCTGGGGGCT | |
| | ATTTGCGGAACTGCGGGCGTGGCGCTGTTGGTGTTGACACCAAACGCAGCG | |
| | CTAGATCCTGTCGGCGTCGCAGCGGGCCTGGCGGGGGCGGTTTCCATGGCG | |
| | TTCGGAACCGTGCTGACCCGCAAGTGGCAACCTCCCGTGCCTCTGCTCACC | |
| | TTTACCGCCTGGCAACTGGCGGCCGGAGGACTTCTGCTCGTTCCAGTAGCT | |
| | TTAGTGTTTGATCCGCCAATCCCGATGCCTACAGGAACCAATGTTCTCGGC | |
| | CTGGCGTGGCTCGGCCTGATCGGAGCGGGTTTAACCTACTTCCTTTGGTTCC | |
| | GGGGGATCTCGCGACTCGAACCTACAGTTGTTTCCTTACTGGGCTTTCTCAG | |
| | CCGGGATGGCGCTAAGAAGCTATTGCCGCCGATCTTCATATGCGGTGTGAA | |
| | ATACCGCACAGATGCGTAAGGAGAAAATACCGCATCAGGCGCTCTTCCGCT | |
| | TCCTCGCTCACTGACTCGCTGCGCTCGGTCGTTCGGCTGCGGCGAGCGGTA | |
| | TCAGCTCACTCAAAGGCGGTAATACGGTTATCCACAGAATCAGGGGATAA | |
| | CGCAGGAAAGAACATGTGAGCAAAAGGCCAGCAAAAGGCCAGGAACCGT | |
| | AAAAAGGCCGCGTTGCTGGCGTTTTTCCATAGGCTCCGCCCCCCTGACGAG | |
| | CATCACAAAAATCGACGCTCAAGTCAGAGGTGGCGAAACCCGACAGGACT | |
| | ATAAAGATACCAGGCGTTTCCCCCTGGAAGCTCCCTCGTGCGCTCTCCTGTT | |
| | CCGACCCTGCCGCTTACCGGATACCTGTCCGCCTTTCTCCCTTCGGGAAGCG | |
| | TGGCGCTTTCTCAATGCTCACGCTGTAGGTATCTCAGTTCGGTGTAGGTCGT | |
| | TCGCTCCAAGCTGGGCTGTGTGCACGAACCCCCCGTTCAGCCCGACCGCTG | |
| | CGCCTTATCCGGTAACTATCGTCTTGAGTCCAACCCGGTAAGACACGACTT | |
| | ATCGCCACTGGCAGCAGCCACTGGTAACAGGATTAGCAGAGCGAGGTATG | |
| | TAGGCGGTGCTACAGAGTTCTTGAAGTGGTGGCCTAACTACGGCTACACTA | |
| | GAAGGACAGTATTTGGTATCTGCGCTCTGCTGAAGCCAGTTACCTTCGGAA | |
| | AAAGAGTTGGTAGCTCTTGATCCGGCAAACAAACCACCGCTGGTAGCGGT | |
| | GGTTTTTTTGTTTGCAAGCAGCAGATTACGCGCAGAAAAAAAGGATATCAA | |
| | GAAGATCCTTTGATCTTTTCTACGGGGTCTGACGCTCAGTGGAACGAAAAC | |
| | TCACGTTAAGGGATTTTGGTCATGAGATTATCAAAAAGGATCTTCACCTAG | |
| | ATCCTTTTAAATTAAAAATGAAGTTTTAAATCAATCTAAAGTATATATGAG | |
| | TAAACTTGGTCTGACAGTTACCAATGCTTAATCAGTGAGGCACCTATCTCA | |
| | GCGATCTGTCTATTTCGTTCATCCATAGTTGCCTGACTCCCCGTCGTGTAGA | |
| | TAACTACGATACGGGAGGGCTTACCATCTGGCCCCAGTGCTGCAATGATAC | |
| | CGCGAGACCCACGCTCACCGGCTCCAGATTTATCAGCAATAAACCAGCCAG | |
| | CCGGAAGGGCCGAGCGCAGAAGTGGTCCTGCAACTTTATCCGCCTCCATCC | |
| | AGTCTATTAAACAAGTGGCAGCAACGGATTCGCAAACCTGTCACGCCTTTT | |
| | GTGCCAAAAGCCGCGCCAGGTTTGCGATCCGCTGTGCCAGGCGTTAGGCGT | |
| | CATATGAAGATTTCGGTGATCCCTGAGCAGGTGGCGGAAACATTGGATGCT | |
| | GAGAACCATTTCATTGTTCGTGAAGTGTTCGATGTGCACCTATCCGACCAA | |
| | GGCTTTGAACTATCTACCAGAAGTGTGAGCCCCTACCGGAAGGATTACATC | |
| | TCGGATGATGACTCTGATGAAGACTCTGCTTGCTATGGCGCATTCATCGAC | |
| | CAAGAGCTTGTCGGGAAGATTGAACTCAACTCAACATGGAACGATCTAGC | |
| | CTCTATCGAACACATTGTTGTGTCGCACACGCACCGAGGCAAAGGAGTCGC | |
| | GCACAGTCTCATCGAATTTGCGAAAAAGTGGGCACTAAGCAGACAGCTCCT | |
| | TGGCATACGATTAGAGACACAAACGAACAATGTACCTGCCTGCAATTTGTA | |
| | CGCAAAATGTGGCTTTACTCTCGGCGGCATTGACCTGTTCACGTATAAAAC | |
| | TAGACCTCAAGTCTCGAACGAAACAGCGATGTACTGGTACTGGTTCTCGGG | |
| | AGCACAGGATGACGCCTAACAATTCATTCAAGCCGACACCGCTTCGCGGCG | |
| | CGGCTTAATTCAGGAGTTAAACATCATGAGGGAAGCGGTGATCGCCGAAG | |
| | TATCGACTCAACTATCAGAGGTAGTTGGCGTCATCGAGCGCCATCTCGAAC | |
| | CGACGTTGCTGGCCGTACATTTGTACGGCTCCGCAGTGGATGGCGGCCTGA | |
| | AGCCACACAGTGATATTGATTTGCTGGTTACGGTGACCGTAAGGCTTGATG | |
| | AAACAACGCGGCGAGCTTTGATCAACGACCTTTTGGAAACTTCGGCTTCCC | |
| | CTGGAGAGAGCGAGATTCTCCGCGCTGTAGAAGTCACCATTGTTGTGCACG | |
| | ACGACATCATTCCGTGGCGTTATCCAGCTAAGCGCGAACTGCAATTTGGAG | |
| | AATGGCAGCGCAATGACATTCTTGCAGGTATCTTCGAGCCAGCCACGATCG | |
| | ACATTGATCTGGCTATCTTGCTGACAAAAGCAAGAGAACATAGCGTTGCCT | |
| | TGGTAGGTCCAGCGGCGGAGGAACTCTTTGATCCGGTTCCTGAACAGGATC | |
| | TATTTGAGGCGCTAAATGAAACCTTAACGCTATGGAACTCGCCGCCCGACT | |
| | GGGCTGGCGATGAGCGAAATGTAGTGCTTACGTTGTCCCGCATTTGGTACA | |
| | GCGCAGTAACCGGCAAAATCGCGCCGAAGGATGTCGCTGCCGACTGGGCA | |
| | ATGGAGCGCCTGCCGGCCCAGTATCAGCCCGTCATACTTGAAGCTAGGCAG | |
| | GCTTATCTTGGACAAGAAGATCGCTTGGCCTCGCGCGCAGATCAGTTGGAA | |
| | GAATTTGTTCACTACGTGAAAGGCGAGATCACCAAGGTAGTCGGCAAATA | |
| | ATGTCTAACAATTCGTTCAAGCCGACGCCGCTTCGCGGCGCGGCTTAACTC | |
| | AAGCGTTAGAGAGCTGGGGAAGACTATGCGCGATCTGTTGAAGGTGGTTCT | |
| | AAGCCTCGTACTTGCGATGGCATCGGGGCAGGCACTTGCTGACCTGCCAAT | |
| | TGTTTTAGTGGATGAAGCTCGTCTTCCCTATGACTACTCCCCATCCAACTAC | |
| | GACATTTCTCCAAGCAACTACGACAACTCCATAAGCAATTACGACAATAGT | |
| | CCATCAAATTACGACAACTCTGAGAGCAACTACGATAATAGTTCATCCAAT | |
| | TACGACAATAGTCGCAACGGAAATCGTAGGCTTATATATAGCGCAAATGG | |
| | GTCTCGCACTTTCGCCGGCTACTACGTCATTGCCAACAATGGGACAACGAA | |
| | CTTCTTTTCCACATCTGGCAAAAGGATGTTCTACACCCCAAAAGGGGGGCG | |
| | CGGCGTCTATGGCGGCAAAGATGGGAGCTTCTGCGGGGCATTGGTCGTCAT | |
| | AAATGGCCAATTTTCGCTTGCCCTGACAGATAACGGCCTGAAGATCATGTA | |
| | TCTAAGCAACTAGCCTGCTCTCTAATAAAATGTTAGGAGCTTGGCTGCCAT | |
| | TTTTGGGGTGAGGCCGTTCGCGGCCGAGGGGCGCAGCCCCTGGGGGGATG | |
| | GGAGGCCCGCGTTAGCGGGCCGGGAGGGTTCGAGAAGGGGGGGCACCCCC | |
| | CTTCGGCGTGCGCGGTCACGCGCCAGGGCGCAGCCCTGGTTAAAAACAAG | |
| | GTTTATAAATATTGGTTTAAAAGCAGGTTAAAAGACAGGTTAGCGGTGGCC | |
| | GAAAAACGGGCGGAAACCCTTGCAAATGCTGGATTTTCTGCCTGTGGACAG | |
| | CCCCTCAAATGTCAATAGGTGCGCCCCTCATCTGTCAGCACTCTGCCCCTCA | |
| | AGTGTCAAGGATCGCGCCCCTCATCTGTCAGTAGTCGCGCCCCTCAAGTGT | |
| | CAATACCGCAGGGCACTTATCCCCAGGCTTGTCCACATCATCTGTGGGAAA | |
| | CTCGCGTAAAATCAGGCGTTTTCGCCGATTTGCGAGGCTGGCCAGCTCCAC | |
| | GTCGCCGGCCGAAATCGAGCCTGCCCCTCATCTGTCAACGCCGCGCCGGGT | |
| | GAGTCGGCCCCTCAAGTGTCAACGTCCGCCCCTCATCTGTCAGTGAGGGCC | |
| | AAGTTTTCCGCGAGGTATCCACAACGCCGGCGGCCGGCCGCGGTGTCTCGC | |
| | ACACGGCTTCGACGGCGTTTCTGGCGCGTTTGCAGGGCCATAGACGGCCGC | |
| | CAGCCCAGCGGCGAGGGCAACCAGCCCGGTGAGCGTCGGAAAGGG | |
| C2 | tcgacgaattaattccaatcccacaaaaatctgagcttaacagcacagtt | 3 |
| hairpin | getcctctcagagcagaatcgggtattcaacaccctcatatcaactacta | |
| cassette | cgttgtgtataacggtccacatgccggtatatacgatgactggggttgta | |
| | caaaggcggcaacaaacggcgttcccggagttgcacacaagaaatttgcc | |
| | actattacagaggcaagagcagcagctgacgcgtacacaacaagtcagca | |
| | aacagacaggttgaacttcatccccaaaggagaagctcaactcaagccca | |
| | agagctttgctaaggccctaacaagcccaccaaagcaaaaagcccactgg | |
| | ctcacgctaggaaccaaaaggcccagcagtgatecagecccaaaagagat | |
| | ctcctttgccccggagattacaatggacgatttcctctatctttacgatc | |
| | taggaaggaagttcgaaggtgaaggtgacgacactatgttcaccactgat | |
| | aatgagaaggttagcctcttcaatttcagaaagaatgctgacccacagat | |
| | ggttagagaggcctacgcagcaggtctcatcaagacgatctacccgagta | |
| | acaatctccaggagatcaaataccttcccaagaaggttaaagatgcagtc | |
| | aaaagattcaggactaattgcatcaagaacacagagaaagacatatttct | |
| | caagatcagaagtactattccagtatggacgattcaaggcttgcttcata | |
| | aaccaaggcaagtaatagagattggagtctctaaaaaggtagttcctact | |
| | gaatctaaggccatgcatggagtctaagattcaaatcgaggatctaacag | |
| | aactcgccgtgaagactggcgaacagttcatacagagtcttttacgactc | |
| | aatgacaagaagaaaatcttcgtcaacatggtggagcacgacactctggt | |
| | ctactccaaaaatgtcaaagatacagtctcagaagaccaaagggctattg | |
| | agacttttcaacaaaggataatttcgggaaacctcctcggattccattgc | |
| | ccagctatctgtcacttcatcgaaaggacagtagaaaaggaaggtggctc | |
| | ctacaaatgccatcattgcgataaaggaaaggctatcattcaagatctct | |
| | ctgccgacagtggtcccaaagatggacccccacccacgaggagcatcgtg | |
| | gaaaaagaagacgttccaaccacgtcttcaaagcaagtggattgatgtga | |
| | catctccactgacgtaagggatgacgcacaatcccactatccttcgcaag | |
| | acccttcctctatataaggaagttcatttcatttggagaggacacg | |
| | ATGTTGAAAGAATATATAACACTGACGATGAGCTCAACAATGCAGGAAGC | |
| | GACGCACCGAAGGAGGAAGAGCCTGGTGATTGAAGGACCATCGAAGACGG | |
| | GTAAAACGCAATGGGCTATATCATTGGGAATACATAACTACTGGTGTGAA | |
| | TCAGTAGACTTCAGTCTATACATAGACGACGCTTTGTACAATATTATTGA | |
| | CGATATACCATTCCAATATTTGCCTTGTAAAAAAGCCTTATTAGGTTGTC | |
| | AATTCAATTACATTGCAAATGAAAAATATCGTCCAAAGCGGAAAATCAAA | |
| | GGAGGGATCCCATCAATTGTTCTTTGTAATCCAGATAACTCTTACTATGA | |
| | TGCAATCACCAATTGGCATGCAACATTCAAGCCGTGGGCTGAAGAAAACA | |
| | TTATCTTTGTAAAAATTGATAGACCGTTGTACTGA | |
| | tcttttttccttttagtataaaatagttaagtgatgttaattagtatgat | |
| | tataataatatagttgttataattgtgaaaaaataatttataaatatatt | |
| | gtttacataaacaacatagtaatgtaaaaaaatatgacaagtgatgtgta | |
| | agacgaagaagataaaagttgagagtaagtatattatttttaatgaattt | |
| | gatcgaacatgtaagatgatatactagcattaatatttgttttaatcata | |
| | atagtaattctagctggtttgatgaattaaatatcaatgataaaatacta | |
| | tagtaaaaataagaataaataaattaaaataatatttttttatgattaat | |
| | agtttattatataattaaatatctataccattactaaatattttagttta | |
| | aaagttaataaatattttgttagaaattccaatctgcttgtaatttatca | |
| | ataaacaaaatattaaataacaagctaaagtaacaaataatatcaaacta | |
| | atagaaacagtaatctaatgtaacaaaacataatctaatgctaatataac | |
| | aaagcgcaagatctatcattttatatagtattattttcaatcaacattct | |
| | tattaatttctaaataatacttgtagttttattaacttctaaatggattg | |
| | actattaattaaatgaattagtcgaacatgaataaacaaggtaacatgat | |
| | agateatgtcattgtgttateattgatettacatttggattg | |
| | TCAGTACAACGGTCTATCAATTTTTACAAAGATAATGTTTTCTTCAGCCC | |
| | ACGGCTTGAATGTTGCATGCCAATTGGTGATTGCATCATAGTAAGAGTTA | |
| | TCTGGATTACAAAGAACAATTGATGGGATCCCTCCTTTGATTTTCCGCTT | |
| | TGGACGATATTTTTCATTTGCAATGTAATTGAATTGACAACCTAATAAGG | |
| | CTTTTTTACAAGGCAAATATTGGAATGGTATATCGTCAATAATATTGTAC | |
| | AAAGCGTCGTCTATGTATAGACTGAAGTCTACTGATTCACACCAGTAGTT | |
| | ATGTATTCCCAATGATATAGCCCATTGCGTTTTACCCGTCTTCGATGGTC | |
| | CTTCAATCACCAGGCTCTTCCTCCTTCGGTGCGTCGCTTCCTGCATTGTT | |
| | GAGCTCATCGTCAGTGTTATATATTCTTTCAACAT | |
| | ctgctttaatgagatatgcgagacgcctatgatcgcatgatatttgcttt | |
| | caattctgttgtgcacgttgtaaaaaacctgagcatgtgtagctcagatc | |
| | cttaccgccggtttcggttcattctaatgaatatatcacccgttactatc | |
| | gtatttttatgaataatattctccgttcaatttactgattgtaccctact | |
| | acttatatgtacaatattaaaatgaaaacaatatattgtgctgaataggt | |
| | ttatagcgacatctatgatagagcgccacaataacaaacaattgcgtttt | |
| | attattacaaatccaattttaaaaaaagcggcagaaccggtcaaacctaa | |
| | aagactgattacataaatcttattcaaatttcaaaaggccccaggggcta | |
| | gtatctacgacacaccgagcggcgaactaataacgttcactgaagggaac | |
| | tccggttccccgccggcgcgcatgggtgagattccttgaagttgagtatt | |
| | ggccgtccgctctaccgaaagttacgggcaccattcaacccggtccagca | |
| | cggcggccgggtaaccgacttgctgccccgagaattatgcagcatttttt | |
| | tggtgtatgtgggccccaaatgaagtgcaggtcaaaccttgacagtgacg | |
| | acaaatcgttgggcgggtccagggcgaattttgcgacaacatgtcgaggc | |
| | tcagcaggacctgcaggcatgcaagctagcttactagtgatgcatattct | |
| | atagtgtcacctaaat | |
| C2 | ATGTTGAAAGAATATATAACACTGACGATGAGCTCAACAATGCAGGAAGC | 4 |
| Hairpin | GACGCACCGAAGGAGGAAGAGCCTGGTGATTGAAGGACCATCGAAGACGG | |
| sense | GTAAAACGCAATGGGCTATATCATTGGGAATACATAACTACTGGTGTGAA | |
| | TCAGTAGACTTCAGTCTATACATAGACGACGCTTTGTACAATATTATTGA | |
| | CGATATACCATTCCAATATTTGCCTTGTAAAAAAGCCTTATTAGGTTGTC | |
| | AATTCAATTACATTGCAAATGAAAAATATCGTCCAAAGCGGAAAATCAAA | |
| | GGAGGGATCCCATCAATTGTTCTTTGTAATCCAGATAACTCTTACTATGA | |
| | TGCAATCACCAATTGGCATGCAACATTCAAGCCGTGGGCTGAAGAAAACA | |
| | TTATCTTTGTAAAAATTGATAGACCGTTGTACTGA | |
| C2 | TCAGTACAACGGTCTATCAATTTTTACAAAGATAATGTTTTCTTCAGCCC | 5 |
| Hairpin | ACGGCTTGAATGTTGCATGCCAATTGGTGATTGCATCATAGTAAGAGTTA | |
| antisense | TCTGGATTACAAAGAACAATTGATGGGATCCCTCCTTTGATTTTCCGCTT | |
| | TGGACGATATTTTTCATTTGCAATGTAATTGAATTGACAACCTAATAAGG | |
| | CTTTTTTACAAGGCAAATATTGGAATGGTATATCGTCAATAATATTGTAC | |
| | AAAGCGTCGTCTATGTATAGACTGAAGTCTACTGATTCACACCAGTAGTT | |
| | ATGTATTCCCAATGATATAGCCCATTGCGTTTTACCCGTCTTCGATGGTC | |
| | CTTCAATCACCAGGCTCTTCCTCCTTCGGTGCGTCGCTTCCTGCATTGTT | |
| | GAGCTCATCGTCAGTGTTATATATTCTTTCAACAT | |
| V2 | tcgacgaattaattccaatcccacaaaaatctgagcttaacagcacagtt | 6 |
| hairpin | getcctctcagagcagaatcgggtattcaacaccctcatatcaactacta | |
| cassette | cgttgtgtataacggtccacatgccggtatatacgatgactggggttgta | |
| | caaaggcggcaacaaacggcgttcccggagttgcacacaagaaatttgcc | |
| | actattacagaggcaagagcagcagctgacgcgtacacaacaagtcagca | |
| | aacagacaggttgaacttcatccccaaaggagaagctcaactcaagccca | |
| | agagctttgctaaggccctaacaagcccaccaaagcaaaaagcccactgg | |
| | ctcacgctaggaaccaaaaggcccagcagtgatecagecccaaaagagat | |
| | ctcctttgccccggagattacaatggacgatttcctctatctttacgatc | |
| | taggaaggaagttcgaaggtgaaggtgacgacactatgttcaccactgat | |
| | aatgagaaggttagcctcttcaatttcagaaagaatgctgacccacagat | |
| | ggttagagaggcctacgcagcaggtctcatcaagacgatctacccgagta | |
| | acaatctccaggagatcaaataccttcccaagaaggttaaagatgcagtc | |
| | aaaagattcaggactaattgcatcaagaacacagagaaagacatatttct | |
| | caagatcagaagtactattccagtatggacgattcaaggcttgcttcata | |
| | aaccaaggcaagtaatagagattggagtctctaaaaaggtagttcctact | |
| | gaatctaaggccatgcatggagtctaagattcaaatcgaggatctaacag | |
| | aactcgccgtgaagactggcgaacagttcatacagagtcttttacgactc | |
| | aatgacaagaagaaaatcttcgtcaacatggtggagcacgacactctggt | |
| | ctactccaaaaatgtcaaagatacagtctcagaagaccaaagggctattg | |
| | agacttttcaacaaaggataatttcgggaaacctcctcggattccattgc | |
| | ccagctatctgtcacttcatcgaaaggacagtagaaaaggaaggtggctc | |
| | ctacaaatgccatcattgcgataaaggaaaggctatcattcaagatctct | |
| | ctgccgacagtggtcccaaagatggacccccacccacgaggagcatcgtg | |
| | gaaaaagaagacgttccaaccacgtcttcaaagcaagtggattgatgtga | |
| | catctccactgacgtaagggatgacgcacaatcccactatccttcgcaag | |
| | acccttcctctatataaggaagttcatttcatttggagaggacacg | |
| | ATGGTAACACTGAACAAACGGAATCGCGTTCTTCCTGAGTGCGATTCCTG | |
| | CAGTTCTAGTGAAAGTTCTTTGAATGATATTGATATTTGTGGTGATGATG | |
| | ATGGGTTAGGGGATGAGGCTTTAGACGCTGGATCCGTTTATTCGTCGTCA | |
| | CAGAAACTGTTAGTTTCTGTGGCTAAAGATGTTCTTTTAGATGACTGTGA | |
| | TTCAACGATATTGGATATATCGTTGCCTTCTGCTTTATGGTTTTTGTCGC | |
| | AAAGATATTTGACTTGTTGTTTGAGGAAAGAATTACTGCCTCTGCCAGGT | |
| | ATATCCGAGAAACAGACTGTTTTATTGCGACAGCTGATTAGGCGTGTCGC | |
| | TCGTCGTCATTGTTTATTTACTTACAAGTGCGAGGAGTGGTTTGAGGGTT | |
| | GTTTGAAGATAAAGAAGGATGGTAATGAAAAAAAGGAGCCGCCAACGGAA | |
| | GCAGAGAAGAAGGCGCAGGACGACTGGGAGGAGTTCTGCCGTAAGGCGGC | |
| | GTGCTCGGCCTCGTAG | |
| | tcttttttccttttagtataaaatagttaagtgatgttaattagtatgat | |
| | tataataatatagttgttataattgtgaaaaaataatttataaatatatt | |
| | gtttacataaacaacatagtaatgtaaaaaaatatgacaagtgatgtgta | |
| | agacgaagaagataaaagttgagagtaagtatattatttttaatgaattt | |
| | gatcgaacatgtaagatgatatactagcattaatatttgttttaatcata | |
| | atagtaattctagctggtttgatgaattaaatatcaatgataaaatacta | |
| | tagtaaaaataagaataaataaattaaaataatatttttttatgattaat | |
| | agtttattatataattaaatatctataccattactaaatattttagttta | |
| | aaagttaataaatattttgttagaaattccaatctgcttgtaatttatca | |
| | ataaacaaaatattaaataacaagctaaagtaacaaataatatcaaacta | |
| | atagaaacagtaatctaatgtaacaaaacataatctaatgctaatataac | |
| | aaagcgcaagatctatcattttatatagtattattttcaatcaacattct | |
| | tattaatttctaaataatacttgtagttttattaacttctaaatggattg | |
| | actattaattaaatgaattagtcgaacatgaataaacaaggtaacatgat | |
| | agatcatgtcattgtgttatcattgatcttacatttggattg | |
| | CTACGAGGCCGAGCACGCCGCCTTACGGCAGAACTCCTCCCAGTCGTCCT | |
| | GCGCCTTCTTCTCTGCTTCCGTTGGCGGCTCCTTTTTTTCATTACCATCC | |
| | TTCTTTATCTTCAAACAACCCTCAAACCACTCCTCGCACTTGTAAGTAAA | |
| | TAAACAATGACGACGAGCGACACGCCTAATCAGCTGTCGCAATAAAACAG | |
| | TCTGTTTCTCGGATATACCTGGCAGAGGCAGTAATTCTTTCCTCAAACAA | |
| | CAAGTCAAATATCTTTGCGACAAAAACCATAAAGCAGAAGGCAACGATAT | |
| | ATCCAATATCGTTGAATCACAGTCATCTAAAAGAACATCTTTAGCCACAG | |
| | AAACTAACAGTTTCTGTGACGACGAATAAACGGATCCAGCGTCTAAAGCC | |
| | TCATCCCCTAACCCATCATCATCACCACAAATATCAATATCATTCAAAGA | |
| | ACTTTCACTAGAACTGCAGGAATCGCACTCAGGAAGAACGCGATTCCGTT | |
| | TGTTCAGTGTTACCAT | |
| | ctgctttaatgagatatgcgagacgcctatgatcgcatgatatttgcttt | |
| | caattctgttgtgcacgttgtaaaaaacctgagcatgtgtagctcagatc | |
| | cttaccgccggtttcggttcattctaatgaatatatcacccgttactatc | |
| | gtatttttatgaataatattctccgttcaatttactgattgtaccctact | |
| | acttatatgtacaatattaaaatgaaaacaatatattgtgctgaataggt | |
| | ttatagcgacatctatgatagagcgccacaataacaaacaattgcgtttt | |
| | attattacaaatccaattttaaaaaaagcggcagaaccggtcaaacctaa | |
| | aagactgattacataaatcttattcaaatttcaaaaggccccaggggcta | |
| | gtatctacgacacaccgagcggcgaactaataacgttcactgaagggaac | |
| | tccggttccccgccggcgcgcatgggtgagattccttgaagttgagtatt | |
| | ggccgtccgctctaccgaaagttacgggcaccattcaacccggtccagca | |
| | cggcggccgggtaaccgacttgctgccccgagaattatgcagcatttttt | |
| | tggtgtatgtgggccccaaatgaagtgcaggtcaaaccttgacagtgacg | |
| | acaaatcgttgggcgggtccagggcgaattttgcgacaacatgtcgaggc | |
| | tcagcaggacctgcaggcatgcaagctagcttactagtgatgcatattct | |
| | atagtgtcacctaaat | |
| V2 | ATGGTAACACTGAACAAACGGAATCGCGTTCTTCCTGAGTGCGATTCCTG | 7 |
| Hairpin | CAGTTCTAGTGAAAGTTCTTTGAATGATATTGATATTTGTGGTGATGATG | |
| sense | ATGGGTTAGGGGATGAGGCTTTAGACGCTGGATCCGTTTATTCGTCGTCA | |
| | CAGAAACTGTTAGTTTCTGTGGCTAAAGATGTTCTTTTAGATGACTGTGA | |
| | TTCAACGATATTGGATATATCGTTGCCTTCTGCTTTATGGTTTTTGTCGC | |
| | AAAGATATTTGACTTGTTGTTTGAGGAAAGAATTACTGCCTCTGCCAGGT | |
| | ATATCCGAGAAACAGACTGTTTTATTGCGACAGCTGATTAGGCGTGTCGC | |
| | TCGTCGTCATTGTTTATTTACTTACAAGTGCGAGGAGTGGTTTGAGGGTT | |
| | GTTTGAAGATAAAGAAGGATGGTAATGAAAAAAAGGAGCCGCCAACGGAA | |
| | GCAGAGAAGAAGGCGCAGGACGACTGGGAGGAGTTCTGCCGTAAGGCGGC | |
| | GTGCTCGGCCTCGTAG | |
| V2 | [symbol] [symbol] [symbol] | 8 |
| Hairpin | [symbol] [symbol] [symbol] | |
| antisense | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] | |

SEQ ID NO: 3: GRBV C2 gene hairpin and recombinant DNA vector cis regulatory sequences, color-coded. Bold are primers used for cloning. Above is the sequence for the RNAfold secondary structure of the C2-i2PDK-antiC2 effector hairpin. p35S (promoter from 35S transcript of Cauliflower Mosaic Virus), C2-SENSE, underlined, SEQ ID NO: 4, in italics, pyruvate orthophosphate dikinase (PDK) intron2 from marigold, Flavaria bidentis (citation: Wesley S V, Helliwell C A, Smith N A, Wang M B, Rouse D T, Liu Q, Gooding P S, Singh S P, AbbottD, Stoutjesdijk P A, Robinson S P, Gleave A P, Green A G, Waterhouse P M. 2001. Construct design for efficient, effective and high-throughput gene silencing in plants. Plant J. 27: 581-90. pHANNIBAL sequence details: NCBI GenBank AJ311872.1), C2-ANTISENSE, underlined italics, SEQ ID NO: 5, and Agrobacterium octopine synthase OCS-transcription terminator, lowercase.

SEQ ID NO: 6: GRBV V2 gene hairpin and recombinant DNA vector cis regulatory sequences, color-coded. Bold are primers used for cloning. Below the sequence is the RNAfold secondary structure of the V2-i2PDK-antiV2 effector hairpin. p35S (promoter from 35S transcript of Cauliflower Mosaic Virus), V2-SENSE, SEQ ID NO: 7, underlined, in italics, pyruvate orthophosphate dikinase (PDK) intron2 from marigold, Flavaria bidentis (citation: Wesley S V, Helliwell C A, Smith N A, Wang M B, Rouse D T, Liu Q, Gooding P S, Singh S P, AbbottD, Stoutjesdijk P A, Robinson S P, Gleave A P, Green A G, Waterhouse P M. 2001. Construct design for efficient, effective and high-throughput gene silencing in plants. Plant J. 27: 581-90. pHANNIBAL sequence details: NCBI GenBank AJ311872.1), V2-ANTISENSE, SEQ ID NO: 8, underlined italics, Agrobacterium octopine synthase OCS-transcription terminator, lowercase.

The disclosures of all patents, patent applications, and publications cited herein are hereby incorporated herein by reference in their entirety, to the extent that they provide exemplary, procedural, or other details supplementary to those set forth herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Sudarshana M R, Gonzalez A, Dave A, Wei A, Smith R, Anderson M M, Walker A M. 2013. Grapevine red blotch-associated virus is widespread in California and U.S. vineyards. Phytopathology 103: S2.140.
2. Cieniewicz E J, Qiu W P, Saldarelli P, Fuchs M. 2020. Believing is seeing: lessons from emerging viruses in grapevine. J Plant Pathol 102: 619-632.
3. Qiu W P, Petersen S M, Howard S. 2020. North American Grape ‘Norton’ is Resistant to Grapevine Vein Clearing Virus. Plant Dis 104: 2051-2053.
4. Fuchs M. 2020. Grapevine viruses: a multitude of diverse species with simple but overall poorly adopted management solutions in the vineyard. J Plant Pathol 102: 643-653.
5. Scholthof K B, Adkins S, Czosnek H, Palukaitis P, Jacquot E, Hohn T, Hohn B, Saunders K, Candresse T, Ahlquist P et al. 2011. Top 10 plant viruses in molecular plant pathology. Mol Plant Pathol 12: 938-954.
6. Zerbini F M, Briddon R W, Idris A, Martin D P, Moriones E, Navas-Castillo J, Rivera-Bustamante R, Roumagnac P, Varsani A, Ictv Report C. 2017. ICTV virus taxonomy profile: Geminiviridae. J General Virol 98: 131-133.
7. Wartig L, Kheyr-Pour A, Noris E, De Kouchkovsky F, Jouanneau F, Gronenborn B, Jupin I. 1997. Genetic analysis of the monopartite Tomato Yellow Leaf Curl geminivirus: roles of V1, V2, and C2 ORFs in viral pathogenesis. Virology 228: 132-140.
8. Vinoth Kumar R, Shivaprasad P V. 2020. Plant-virus-insect tritrophic interactions: insights into the functions of geminivirus virion-sense strand genes. Proc Biol Sci 287: 1846.
9. Calvi B L. 2011. Effects of red-leaf disease on Cabernet Sauvignon at the Oakville experimental vineyard and mitigation by harvest delay and crop adjustment. MSc thesis University of California, Davis.
10. Yepes L M, Cieniewicz E, Krenz B, McLane H, Thompson J R, Perry K L, Fuchs M. 2018. Causative role of Grapevine Red Blotch Virus in Red Blotch Disease. Phytopathology 108: 902-909.
11. Buchs N, Braga-Lagache S, Uldry A-C, Brodard J, Debonneville C, Reynard J-S, Heller M. 2018. Absolute quantification of Grapevine Red Blotch Virus in grapevine leaf and petiole tissues by proteomics. Front Plant Sci 9: 1735.
12. Sudarshana M R, Perry K L, Fuchs M F. 2015. Grapevine Red Blotch-Associated Virus, an emerging threat to the grapevine industry. Phytopathology 105: 1026-1032.
13. Yamakawa T, Kato S, Ishida K, Kodama T, Minoda Y. 1983. Production of anthocyanins by Vitis cells in suspension culture. Agric Biol Chem 47: 2185-2191.
14. Oberhoster A, Girardello R C, Lerno L A, Eridon S, Cooper M L Y, Smith R H, Brenneman C A, Heymann H, Sokolowsky M. 2016. Impact of Red Blotch Disease on grape and wine composition. Workshop on Recent Advances in Viticulture and Enology, U C Davis, Dec. 9, 2016 available at http://ucanr.edu/repository/fileaccess.cfm?article=162938&p=YQCEFO.
15. Bowen P, Bogdanoff C, Poojari S, Usher K, Lowery T, Urbez-Torres J R. 2020. Effects of Grapevine Red Blotch Disease on Cabernet franc vine physiology, bud hardiness, and fruit and wine quality. Amer J Enol Vitic 71: 308-318.
16. Wallis C M, Sudarshana M R. 2016. Effects of Grapevine red blotch-associated virus (GRBaV) infection on foliar metabolism of grapevines. Canadian J Plant Pathol 38: 358-366.
17. Rwahnih M A, Rowhani A, Golino D A, Islas C M, Preece J E, Sudarshana M R. 2015. Detection and genetic diversity of Grapevine red blotch-associated virus isolates in table grape accessions in the National Clonal Germplasm Repository in California. Canadian J Plant Pathol 37: 130-135.
18. Girardello R C, Cooper M L, Lerno L A, Brenneman C, Eridon S, Sokolowsky M, Heymann H, Oberholster A. 2020. Impact of Grapevine Red Blotch Disease on Cabernet Sauvignon and Merlot wine composition and sensory attributes. Molecules 25: 26.
19. Girardello R C, Rich V, Smith R J, Brenneman C, Heymann H, Oberholster A. 2020. The impact of Grapevine Red Blotch Disease on Vitis vinifera L. Chardonnay grape and wine composition and sensory attributes over three seasons. J Sci Food Agric 100: 1436-1447.
20. Martinez-Luscher J, Plank C M, Brillante L, Cooper M L, Smith R J, Al-Rwahnih M, Yu R Z, Oberholster A, Girardello R, Kurtural S K. 2019. Grapevine Red Blotch Virus may reduce carbon translocation leading to impaired grape berry ripening. J Agric Food Chem 67: 2437-2448.
21. Ricketts K D, Gomez M I, Fuchs M F, Martinson T E, Smith R J, Cooper M L, Moyer M M, Wise A. 2017. Mitigating the economic impact of Grapevine Red Blotch: optimizing disease management strategies in U.S. vineyards. Amer J Enology Vitic 68: 127-135.
22. Levin A D, Achala N K C. 2020. Water deficits do not improve fruit quality in Grapevine Red Blotch Virus-infected grapevines (Vitis vinifera L.). Front Plant Sci 11: 13.
23. Setiono F J, Chatterjee D, Fuchs M, Perry K L, Thompson J R. 2018. The distribution and detection of Grapevine Red Blotch Virus in its host depend on time of sampling and tissue type. Plant Dis 102: 2187-2193.
24. Cieniewicz E, Flasco M, Brunelli M, Onwumelu A, Wise A, Fuchs M F. 2019. Differential spread of Grapevine Red Blotch Virus in California and New York vineyards. Phytobiomes J 3: 203-211.
25. Krenz B, Thompson J R, McLane H L, Fuchs M, Perry K L. 2014. Grapevine red blotch-associated virus is widespread in the United States. Phytopathology 104: 1232-1240.
26. Cieniewicz E J, Pethybridge S J, Gorny A, Madden L V, McLane H, Perry K L, Fuchs M. 2017. Spatiotemporal spread of grapevine red blotch-associated virus in a California vineyard. Virus Res 241: 156-162.
27. Dalton D T, Hilton R J, Kaiser C, Daane K M, Sudarshana M R, Vo J, Zalom F G, Buser J Z, Walton V M. 2019. Spatial associations of vines infected with Grapevine Red Blotch Virus in Oregon vineyards. Plant Dis 103: 1507-1514.
28. Bander B W, Zalom F G, Jayanth M, Sudarshana M R. 2016. Phylogeny of geminivirus coat protein sequences and digital PCR aid in identifying Spissistilus festinus as a vector of grapevine red blotch-associated virus. Phytopathology 106: 1223-1230.
29. Poojari S, Alabi O J, Fofanov V Y, Naidu R A. 2013. A leafhopper-transmissible DNA virus with novel evolutionary lineage in the family Geminiviridae implicated in Grapevine Redleaf Disease by next-generation sequencing. PLoS ONE 8: e64194.
30. Cieniewicz E J, Pethybridge S J, Loeb G, Perry K, Fuchs M. 2018. Insights into the ecology of Grapevine Red Blotch Virus in a diseased vineyard. Phytopathology 108: 94-102.
32. Kron C R, Sisterson M S. 2020. Identification of nonhost cover crops of the three-cornered alfalfa hopper (Spissistilus festinus). Amer J Enol Vitic 71: 175-180.
33. Vargas-Asencio J, Liou H, Perry K L, Thompson J R. 2019. Evidence for the splicing of grablovirus transcripts reveals a putative novel open reading frame. J Gen Virol 100: 709-720.
34. Dekker E L, Woolston C J, Xue Y B, Cox B, Mullineaux P M. 1991. Transcript mapping reveals different expression strategies for the bicistronic RNAs of the geminivirus wheat dwarf virus. Nucl Acids Res 19: 4075-4081.
35. Wright E A, Heckel T, Groenendijk J, Davies J W, Boulton M I. 1997. Splicing features in maize streak virus virion- and complementary-sense gene expression. Plant J 12: 1285-1297.
36. Guo T W, Vimalesvaran D, Thompson J R, Perry K L, Krenz B. 2015. Subcellular localization of Grapevine Red Blotch-associated Virus O RFs V2 and V3. Virus Genes 51: 156-158.
37. Blanco-Ulate B, Hopfer H, Figueroa-Balderas R, Ye Z, Rivero R M, Albacete A, Perez-Alfocea F, Koyama R, Anderson M M, Smith R J et al. 2017. Red blotch disease alters grape berry development and metabolism by interfering with the transcriptional and hormonal regulation of ripening. J Exp Bot 68: 1225-1238.
38. Trinks D, Rajeswaran R, Shivaprasad P V, Akbergenov R, Oakeley E J, Veluthambi K, Hohn T, Pooggin M M. 2005. Suppression of RNA silencing by a geminivirus nuclear protein, AC2, correlates with transactivation of host genes. J Virol 79: 2517-2527.
39. Bisaro D M. 2006. Silencing suppression by geminivirus proteins. Virology 344: 158-168.
40. Wang H, Hao L, Shung C-Y, Sunter G, Bisaro D M. 2003. Adenosine kinase is inactivated by geminivirus AL2 and L2 proteins. Plant Cell 15: 3020-3032.
41. Yong Chung H, Lacatus G, Sunter G. 2014. Geminivirus A L2 protein induces expression of, and interacts with, a calmodulin-like gene, an endogenous regulator of gene silencing. Virology 460-461: 108-118.
42. Hao L, Wang H, Sunter G, Bisaro D M. 2003. Geminivirus A L2 and L2 proteins interact with and inactivate SNF1 kinase. Plant Cell 15: 1034-1048.
43. Zhang Z, Chen H, Huang X, Xia R, Zhao Q, Lai J, Teng K, Li Y, Liang L, Du Q et al. 2011. BSCTV C2 attenuates the degradation of SAMDC1 to suppress DNA methylation-mediated gene silencing in Arabidopsis. Plant Cell 23: 273-288.
44. Buchmann R C, Asad S, Wolf J N, Mohannath G, Bisaro D M. 2009. Geminivirus A L2 and L2 proteins suppress transcriptional gene silencing and cause genome-wide reductions in cytosine methylation. J Virol 83: 5005-5013.
45. Castillo-Gonzalez C, Liu X, Huang C, Zhao C, Ma Z, Hu T, Sun F, Zhou Y, Zhou X, Wang X-J et al. 2015. Geminivirus-encoded TrAP suppressor inhibits the histone methyltransferase SUVH4/KYP to counter host defense. eLife 4: e06671.
46. Re D A, Manavella P A. 2015. Caught in a TrAP. eLife 4: e11509-e11509.
47. Baliji S, Lacatus G, Sunter G. 2010. The interaction between geminivirus pathogenicity proteins and adenosine kinase leads to increased expression of primary cytokinin responsive genes. Virology 402: 238-247.
48. Lozano-Duran R, Rosas-Diaz T, Gusmaroli G, Luna A P, Taconnat L, Deng X W, Bejarano E R. 2011. Geminiviruses subvert ubiquitination by altering CSN-mediated derubylation of SCF E3 ligase complexes and inhibit jasmonate signaling in Arabidopsis thaliana. Plant Cell 23: 1014-1032.
49. Rosas-Diaz T, Macho A P, Beuzón C R, Lozano-Duran R, Bejarano E R. 2016. The C2 protein from the geminivirus Tomato Yellow Leaf Curl Sardinia Virus decreases sensitivity to jasmonates and suppresses jasmonate-mediated defences. Plants 5: 8.
50. Sun Y-W, Tee C-S, Ma Y-H, Wang G, Yao X-M, Ye J. 2015. Attenuation of histone methyltransferase KRYPTONITE-mediated transcriptional gene silencing by Geminivirus. Sci Rep 5: 16476.
51. Kumar V, Mishra S K, Rahman J, Taneja J, Sundaresan G, Mishra N S, Mukherjee S K. 2015. Mungbean yellow mosaic Indian virus encoded AC2 protein suppresses RNA silencing by inhibiting Arabidopsis RDR6 and AGO1 activities. Virology 486: 158-172.
52. Luan J B, Yao D M, Zhang T, Walling L L, Yang M, Wang Y J, Liu S S. 2013. Suppression of terpenoid synthesis in plants by a virus promotes its mutualism with vectors. Ecol Lett 16: 390-398.
53. Li P, Liu C, Deng W H, Yao D M, Pan L L, Li Y Q, Liu Y Q, Liang Y, Zhou X P, Wang W. 2019. Plant begomoviruses subvert ubiquitination to suppress plant defenses against insect vectors. PLoS Pathogens 15: e1007607.
54. Tu Y C, Tsai W S, Wei J Y, Chang K Y, Tien C C, Hsiao H Y, Fu S F. 2017. The C2 protein of tomato leaf curl Taiwan virus is a pathogenicity determinant that interferes with expression of host genes encoding chromomethylases. Physiol Plant 161: 515-531.
55. Chellappan P, Vanitharani R, Fauquet C M. 2005. MicroRNA-binding viral protein interferes with Arabidopsis development. Proc Natl Acad Sci USA 102: 10381-10386.
56. Wang Y, Dang M, Hou H, Mei Y, Qian Y, Zhou X. 2014. Identification of an RNA silencing suppressor encoded by a mastrevirus. J Gen Virol 95: 2082-2088.
57. Wang B, Yang X, Wang Y, Xie Y, Zhou X. 2018. Tomato Yellow Leaf Curl Virus V2 Interacts with host Histone Deacetylase 6 to suppress methylation-mediated transcriptional gene silencing in plants. J Virol 92: e00036-00018.
58. Glick E, Zrachya A, Levy Y, Mett A, Gidoni D, Belausov E, Citovsky V, Gafni Y. 2008. Interaction with host SGS3 is required for suppression of RNA silencing by tomato Yellow Leaf Curl Virus V2 protein. Proc Natl Acad Sci USA 105: 157-161.
59. Wang L P, Ding Y, He L, Zhang G P, Zhu J K, Lozano-Duran R. 2020. A virus-encoded protein suppresses methylation of the viral genome through its interaction with AGO4 in the Cajal body. Elife 9: 21.
60. Wang Y, Wu Y, Gong Q, Ismayil A, Yuan Y, Lian B, Jia Q, Han M, Deng H, Hong Y et al. 2019. Geminiviral V2 protein suppresses transcriptional gene silencing through interaction with AGO4. J Virol 93.
61. Sun S, Hu Y, Jiang G, Tian Y, Ding M, Yu C, Zhou X, Qian Y. 2020. Molecular characterization and genomic function of Grapevine Geminivirus A. Front Microbiol 11: 555194.
62. Yang X, Ren Y, Sun S, Wang D, Zhang F, Li D, Li S, Zhou X. 2018. Identification of the potential virulence factors and RNA silencing suppressors of Mulberry Mosaic Dwarf-Associated Geminivirus. Viruses 10: 472.
63. Vaucheret H. 2006. Post-transcriptional small RNA pathways in plants: mechanisms and regulations. Genes Dev 20: 759-771.
64. Weiberg A, Bellinger M, Jin H L. 2015. Conversations between kingdoms: small RNAs. Curr Opin Biotechnol 32: 207-215.
65. Fitch M M M, Manshardt R M, Gonsalves D, Slightom J L, Sanford J C. 1992. Virus resistant papaya plants derived from tissues bombarded with the coat protein gene of Papaya Ringspot Virus. Bio/Technology 10: 1466-1472.
66. Ferreira S A, Pitz K Y, Manshardt R, Zee F, Fitch M, Gonsalves D. 2002. Virus coat protein transgenic papaya provides practical control of Papaya ringspot virus in Hawaii. Plant Dis 86: 101-105.
67. Jongedijk E, Huisman M J, Cornelissen B J C. 1993. Argonic performance and field resistance of genetically modified, virus-resistant potato plants. Sem Virol 4: 407-416.
68. Hu J S, Pang S Z, Nagpala P G, Siemieniak D R, Slightom J L, Gonsalves D. 1993. The coat protein genes of squash mosaic virus: cloning, sequence analysis, and expression in tobacco protoplasts. Arch Virol 130: 17-31.
69. Kunik T, Salomon R, Zamir D, Navot N, Zeidan M, Michelson I, Gafni Y, Czosnek H. 1994. Transgenic tomato plants expressing the Tomato Yellow Leaf Curl Virus capsid protein are resistant to the virus. Bio/Technology 12: 500-504.
70. Prins M, Laimer M, Noris E, Schubert J, Wassenegger M, Tepfer M. 2008. Strategies for antiviral resistance in transgenic plants. Mol Plant Pathol 9: 73-83.
71. Shepherd D N, Martin D P, Thomson J A. 2009. Transgenic strategies for developing crops resistant to geminiviruses. Plant Sci 176: 1-11.
72. Vanderschuren H, Stupak M, Fütterer J, Gruissem W, Zhang P. 2007. Engineering resistance to geminiviruses—review and perspectives. Plant Biotech J 5: 207-220.
73. Brunetti A, Tavazza R, Noris E, Lucioli A, Accotto G P, Tavazza M. 2001. Transgenically expressed T-Rep of Tomato Yellow Leaf Curl Sardinia Virus acts as a trans-dominant-negative mutant, inhibiting viral transcription and replication. J Virol 75: 10573-10581.
74. Hanson S F, Maxwell D P. 1999. trans-Dominant inhibition of geminiviral DNA replication by Bean Golden Mosaic Geminivirus rep gene mutants. Phytopathology 89: 480-486.
75. Chatterji A, Beachy R N, Fauquet C M. 2001. Expression of the oligomerization domain of the replication-associated protein (Rep) of Tomato Leaf Curl New Delhi Virus interferes with DNA accumulation of heterologous geminiviruses. J Biol Chem 276: 25631-25638.
76. Lucioli A, Noris E, Brunetti A, Tavazza R, Ruzza V, Castillo A G, Bejarano E R, Accotto G P, Tavazza M. 2003. Tomato Yellow Leaf Curl Sardinia Virus Rep-derived resistance to homologous and heterologous geminiviruses occurs by different mechanisms and is overcome if virus-mediated transgene silencing is activated. J Virol 77: 6785-6798.
77. Hayes R J, Buck K W. 1989. Replication of tomato golden mosaic virus DNA B in transgenic plants expressing open reading frames (ORFs) of DNA A: requirement of ORF AL2 for production of single-stranded DNA. Nucl Acids Res 17: 10213-10222.
78. Hanley-Bowdoin L, Elmer J S, Rogers S G. 1990. Expression of functional replication protein from tomato golden mosaic virus in transgenic tobacco plants. Proc Natl Acad Sci USA 87: 1446-1450.
79. Padidam M, Beachy R N, Fauquet C M. 1999. A phage single-stranded DNA (ssDNA) binding protein complements ssDNA accumulation of a geminivirus and interferes with viral movement. J Virol 73: 1609-1616.
80. Sunitha S, Marian D, Hohn B, Veluthambi K. 2011. Antibegomoviral activity of the agrobacterial virulence protein VirE2. Virus Genes 43: 445.
81. Resmi T R, Hohn T, Hohn B, Veluthambi K. 2015. The Agrobacterium tumefaciens Ti plasmid virulence gene virE2 reduces Sri Lankan Cassava Mosaic Virus infection in transgenic Nicotiana benthamiana plants. Viruses 7: 2641-2653.
82. Yousaf S, Rasool G, Amin I, Mansoor S, Saeed M. 2015. Evaluation of the resistance against begomoviruses imparted by the single-stranded DNA binding protein VirE2. Pakistan J Agric Sci 52: 887-893.
83. Day A G, Bejarano E R, Buck K W, Burrell M, Lichtenstein C P. 1991. Expression of an antisense viral gene in transgenic tobacco confers resistance to the DNA virus tomato golden mosaic virus. Proc Natl Acad Sci USA 88: 6721-6725.
84. Aragão FJL, Ribeiro S G, Barros L M G, Brasileiro A C M, Maxwell D P, Rech E L, Faria J C. 1998. Transgenic beans (Phaseolus vulgaris L.) engineered to express viral antisense RNAs show delayed and attenuated symptoms to bean golden mosaic geminivirus. Mol Breeding 4: 491-499.
85. Bendahmane M, Gronenborn B. 1997. Engineering resistance against tomato yellow leaf curl virus (TYLCV) using antisense RNA. Plant Mol Biol 33: 351-357.
86. Yang Y, Sherwood T A, Patte C P, Hiebert E, Polston J E. 2004. Use of Tomato yellow leaf curl virus (TYLCV) Rep gene sequences to engineer TYLCV resistance in tomato. Phytopathology 94: 490-496.
87. Asad S, Haris W A A, Bashir A, Zafar Y, Malik K A, Malik N N, Lichtenstein C P. 2003. Transgenic tobacco expressing geminiviral RNAs are resistant to the serious viral pathogen causing cotton leaf curl disease. Arch Virol 148: 2341-2352.
88. Pooggin M, Shivaprasad P V, Veluthambi K, Hohn T. 2003. RNAi targeting of DNA virus in plants. Nat Biotech 21: 131-132.
89. Vanitharani R, Chellappan P, Fauquet C M. 2003. Short interfering RNA-mediated interference of gene expression and viral DNA accumulation in cultured plant cells. Proc Natl Acad Sci USA 100: 9632-9636.
90. Owor B E, Martin D P, Rybicki E P, Thomson J A, Bezuidenhout M E, Lakay F M, Shepherd D N. 2011. A rep-based hairpin inhibits replication of diverse maize streak virus isolates in a transient assay. J Gen Virol 92: 2458-2465.
91. Fuentes A, Ramos P L, Fiallo E, Callard D, Sanchez Y, Peral R, Rodriguez R, Pujol M. 2006. Intron-hairpin RNA derived from replication associated protein C1 gene confers immunity to Tomato Yellow Leaf Curl Virus infection in transgenic tomato plants. Transgenic Res 15: 291-304.
92. Aragão FJL, Faria J C. 2009. First transgenic geminivirus-resistant plant in the field. Nat Biotech 27: 1086-1088.
93. Bonfim K, Faria J C, Nogueira EOPL, Mendes ÉA, Aragão FJL. 2007. RNAi-Mediated resistance to Bean golden mosaic virus in genetically engineered common bean (Phaseolus vulgaris). Mol Plant-Microbe Interact 20: 717-726.
94. Vanderschuren H, Alder A, Zhang P, Gruissem W. 2009. Dose-dependent RNAi-mediated geminivirus resistance in the tropical root crop cassava. Plant Mol Biol 70: 265-272.
95. Vanderschuren H, Akbergenov R, Pooggin M M, Hohn T, Gruissem W, Zhang P. 2007. Transgenic cassava resistance to African cassava mosaic virus is enhanced by viral DNA-A bidirectional promoter-derived siRNAs. Plant Mol Biol 64: 549-557.
96. Zrachya A, Glick E, Levy Y, Arazi T, Citovsky V, Gafni Y. 2007. Suppressor of RNA silencing encoded by Tomato yellow leaf curl virus-Israel. Virology 358: 159-165.
97. Praveen S, Ramesh S V, Mishra A K, Koundal V, Palukaitis P. 2010. Silencing potential of viral derived RNAi constructs in Tomato leaf curl virus-AC4 gene suppression in tomato. Transgenic Res 19: 45-55.
98. Sunitha S, Shanmugapriya G, Balamani V, Veluthambi K. 2013. Mungbean yellow mosaic virus (MYMV) AC4 suppresses post-transcriptional gene silencing and an AC4 hairpin RNA gene reduces MYMV DNA accumulation in transgenic tobacco. Virus Genes 46: 496-504.
99. Shanmugapriya G, Das S S, Veluthambi K. 2015. Transgenic tobacco plants expressing siRNA targeted against the Mungbean yellow mosaic virus transcriptional activator protein gene efficiently block the viral DNA accumulation. Virus Disease 26: 55-61.
100. Luo Q-J, Mittal A, Jia F, Rock C D. 2012. An autoregulatory feedback loop involving PAP1 and TAS4 in response to sugars in Arabidopsis. Plant Mol Biol 80: 117-129.
101. Rock C D. 2013. Trans-acting small interfering RNA4: key to nutraceutical synthesis in grape development? Trends Plant Sci 18: 601-610.
102. Pantaleo V, Szittya G, Moxon S, Miozzi L, Moulton V, Dalmay T, Burgyan J. 2010. Identification of grapevine microRNAs and their targets using high-throughput sequencing and degradome analysis. Plant J 62: 960-976.
103. Sunitha S, Loyola R, Alcalde J A, Arce-Johnson P, Matus J T, Rock C D. 2019. The role of U V-B light on small RNA activity during grapevine berry development. G3: Genes|Genomes|Genetics 9: 769-787.
104. Rock C D, Zeevaart J A. 1991. The aba mutant of Arabidopsis thaliana is impaired in epoxy-carotenoid biosynthesis. Proc Natl Acad Sci USA 88: 7496-7499.
105. Kovacs L G, Hanami H, Fortenberry M, Kaps M L. 2001. Latent infection by leafroll agent GLRaV-3 is linked to lower fruit quality in French-American hybrid grapevines Vidal blanc and St. Vincent. American Journal of Enology and Viticulture 52: 254-259.
106. Syller J. 2012. Facilitative and antagonistic interactions between plant viruses in mixed infections. Mol Plant Pathol 13: 204-216.
107. Blaisdell G K, Zhang S, Rowhani A, Klaassen V, Cooper M L, Daane K M, Almeida R P P. 2020. Trends in vector-borne transmission efficiency from coinfected hosts: Grapevine leafroll-associated virus-3 and Grapevine virus A. Eur J Plant Pathol 156: 1163-1167.
108. Aguilar E, Almendral D, Allende L, Pacheco R, Chung B N, Canto T, Tenllado F. 2015. The P25 protein of Potato Virus X (PVX) is the main pathogenicity determinant responsible for systemic necrosis in PVX-associated synergisms. J Virol 89: 2090-2103.
109. Liu Y, Teng C, Xia R, Meyers B C. 2020. PhasiRNAs in plants: their biogenesis, genic sources, and roles in stress responses, development, and reproduction. Plant Cell 32: 3059-3080.
110. Padmanabhan C, Zhang X M, Jin H L. 2009. Host small RNAs are big contributors to plant innate immunity. Curr Opin Plant Biol 12: 465-472.
111. Zhang X M, Zhao H W, Gao S, Wang W C, Katiyar-Agarwal S, Huang H D, Raikhel N, Jin H L. 2011. Arabidopsis Argonaute 2 regulates innate immunity via miRNA393*-mediated silencing of a Golgi-localized SNARE gene, MEMB12. Mol Cell 42: 356-366.
112. Wang X-B, Jovel J, Udomporn P, Wang Y, Wu Q, Li W-X, Gasciolli V, Vaucheret H, Ding S-W. 2011. The 21-nucleotide, but not 22-nucleotide, viral secondary small interfering RNAs direct potent antiviral defense by two cooperative Argonautes in Arabidopsis thaliana. Plant Cell 23: 1625-1638.
113. Schott G, Mari-Ordonez A, Himber C, Alioua A, Voinnet O, Dunoyer P. 2012. Differential effects of viral silencing suppressors on siRNA and miRNA loading support the existence of two distinct cellular pools of ARGONAUTE1. EMBO J 31: 2553-2565.
114. Li F, Pignatta D, Bendix C, Brunkard J O, Cohn M M, Tung J, Sun H, Kumar P, Baker B. 2012. MicroRNA regulation of plant innate immune receptors. Proc Natl Acad Sci USA 109: 1790-1795.
115. Shivaprasad P V, Chen H M, Patel K, Bond D M, Santos B, Baulcombe D C. 2012. A microRNA superfamily regulates nucleotide binding site-leucine-rich repeats and other mRNAs. Plant Cell 24: 859-874.
116. Seifi A. 2011. Write ‘systemic small RNAs’: read ‘systemic immunity’. Funct Plant Biol 38: 747-752.
117. Boyko A, Kovalchuk I. 2011. Genetic and epigenetic effects of plant-pathogen interactions: an evolutionary perspective. Mol Plant 4: 1014-1023.
118. Mourrain P, Beclin C, Elmayan T, Feuerbach F, Godon C, Morel J B, Jouette D, Lacombe A M, Nikic S, Picault N et al. 2000. Arabidopsis SGS2 and SGS3 genes are required for posttranscriptional gene silencing and natural virus resistance. Cell 101: 533-542.
119. Wang Z, Hardcastle T J, Canto Pastor A, Yip W H, Tang S, Baulcombe D C. 2018. A novel DCL2-dependent miRNA pathway in tomato affects susceptibility to RNA viruses. Genes Dev 32: 1155-1160.
120. Zhai J, Jeong D-H, De Paoli E, Park S, Rosen B D, Li Y, Gonzalez A J, Yan Z, Kitto S L, Grusak M A et al. 2011. MicroRNAs as master regulators of the plant N B-LRR defense gene family via the production of phased, trans-acting siRNAs. Genes Dev 25: 2540-2553.
121. Song Q-X, Liu Y-F, Hu X-Y, Zhang W-K, Ma B, Chen S-Y, Zhang J-S. 2011. Identification of miRNAs and their target genes in developing soybean seeds by deep sequencing. BMC Plant Biol 11: 5.
122. Li H, Deng Y, Wu T, Subramanian S, Yu O. 2010. Misexpression of miR482, miR1512, and miR1515 increases soybean nodulation. Plant Physiol 153: 1759-1770.
123. Axtell M J, Jan C, Rajagopalan R, Bartel D P. 2006. A two-hit trigger for siRNA biogenesis in plants. Cell 127: 565-577.
124. Hellens R P, Edwards E A, Leyland N R, Bean S, Mullineaux P M. 2000. pGreen: a versatile and flexible binary Ti vector for Agrobacterium-mediated plant transformation. Plant Mol Biol 42: 819-832.
125. Ruiz M T, Voinnet O, Baulcombe D C. 1998. Initiation and maintenance of virus-induced gene silencing. Plant Cell 10: 937-946.
126. Anandalakshmi R, Pruss G J, Ge X, Marathe R, Mallory A C, Smith T H, Vance V B. 1998. A viral suppressor of gene silencing in plants. Proc Natl Acad Sci USA 95: 13079-13084.
127. Johnson N R, Yeoh J M, Coruh C, Axtell M J. 2016. Improved placement of multi-mapping small RNAs. G3: Genes|Genomes|Genetics 6: 2103-2111.
128. Yang Y, Liu T, Shen D, Wang J, Ling X, Hu Z, Chen T, Hu J, Huang J, Yu W et al. 2019. Tomato yellow leaf curl virus intergenic siRNAs target a host long noncoding RNA to modulate disease symptoms. PLOS Pathogens 15: e1007534.
129. Shimura H, Pantaleo V, Ishihara T, Myojo N, Inaba J-i, Sueda K, Burgyan J, Masuta C. 2011. A viral satellite RNA induces yellow symptoms on tobacco by targeting a tene involved in chlorophyll biosynthesis using the RNA silencing machinery. PLOS Pathogens 7: e1002021.
130. Smith N A, Eamens A L, Wang M-B. 2011. Viral small interfering RNAs target host genes to mediate disease symptoms in plants. PLOS Pathogens 7: e1002022.
131. Brilli M, Asquini E, Moser M, Bianchedi P L, Perazzolli M, Si-Ammour A. 2018. A multi-omics study of the grapevine-downy mildew (Plasmopara viticola) pathosystem unveils a complex protein coding- and noncoding-based arms race during infection. Sci Rep 8: 757.
132. Chitarra W, Pagliarani C, Abba S, Boccacci P, Birello G, Rossi M, Palmano S, Marzachi C, Perrone I, Gambino G. 2018. miRVIT: A novel miRNA database and its application to uncover Vitis responses to Flavescence dorée infection. Front Plant Sci 9: 1034.
133. Reynard J-S, Brodard J, Dubuis N, Zufferey V, Schumpp O, Schaerer S, Gugerli P. 2017. Grapevine red blotch virus: absence in Swiss vineyards and analysis of potential detrimental effect on viticultural performance. Plant Dis 102: 651-655.
134. Love M I, Huber W, Anders S. 2014. Moderated estimation of fold change and dispersion for RNA-seq data with DESeq2. Genome Biol 15: 550.
135. Usadel B, Poree F, Nagel A, Lohse M, Czedik-Eysenberg A, Stitt M. 2009. A guide to using MapMan to visualize and compare Omics data in plants: a case study in the crop species, maize. Plant Cell Environ 32: 1211-1229.
136. Valluru R, Van den Ende W. 2011. Myo-inositol and beyond—Emerging networks under stress. Plant Sci 181: 387-400.
137. Li H, Ma D, Jin Y, Tu Y, Liu L, Leng C, Dong J, Wang T. 2015. Helper component-proteinase enhances the activity of 1-deoxy-D-xylulose-5-phosphate synthase and promotes the biosynthesis of plastidic isoprenoids in Potato virus Y-infected tobacco. Plant Cell Environ 38: 2023-2034.
138. Bürstenbinder K, Mitra D, Quegwer J. 2017. Functions of IQD proteins as hubs in cellular calcium and auxin signaling: A toolbox for shape formation and tissue-specification in plants? Plant Signal Behav 12: e1331198.
139. Anandalakshmi R, Marathe R, Ge X, Herr J M, Mau C, Mallory A, Pruss G, Bowman L, Vance V B. 2000. A calmodulin-related protein that suppresses posttranscriptional gene silencing in plants. Science 290: 142-144.
140. Jeon E J, Tadamura K, Murakami T, Inaba J-i, Kim B M, Sato M, Atsumi G, Kuchitsu K, Masuta C, Nakahara K S. 2017. rgs-CaM detects and counteracts viral RNA silencing suppressors in plant immune priming. J Virol 91: e00761-00717.
141. Nakahara K S, Masuta C, Yamada S, Shimura H, Kashihara Y, Wada T S, Meguro A, Goto K, Tadamura K, Sueda K et al. 2012. Tobacco calmodulin-like protein provides secondary defense by binding to and directing degradation of virus RNA silencing suppressors. Proc Natl Acad Sci USA 109: 10113-10118.
142. Andika I B, Maruyama K, Sun L, Kondo H, Tamada T, Suzuki N. 2015. Differential contributions of plant Dicer-like proteins to antiviral defences against potato virus X in leaves and roots. Plant J 81: 781-793.
143. Qu F, Ye X H, Morris T J. 2008. Arabidopsis DRB4, AG01, AG07, and RDR6 participate in a DCL4-initiated antiviral RNA silencing pathway negatively regulated by DCL1. Proc Natl Acad Sci USA 105: 14732-14737.
144. Adenot X, Elmayan T, Lauressergues D, Boutet S, Bouche N, Gasciolli V, Vaucheret H. 2006. DRB4-dependent TAS3 trans-acting siRNAs control leaf morphology through AGO7. Curr Biol 16: 927-932.
145. Brosseau C, Moffett P. 2015. Functional and genetic analysis identify a role for Arabidopsis ARGONAUTES in antiviral RNA silencing. Plant Cell 27: 1742-1754.
146. Mann R S, Ali J G, Hermann S L, Tiwari S, Pelz-Stelinski K S, Alborn H T, Stelinski L L. 2012. Induced release of a plant-defense volatile ‘deceptively’ attracts insect vectors to plants infected with a bacterial pathogen. PLoS Pathogens 8: e1002610.
147. Robacker D C, Massa M J, Sacchetti P, Bartelt R J. 2011. A novel attractant for Anastrepha ludens (Diptera: Tephritidae) from a Concord grape product. J Econom Entomol 104: 1195-1203.
148. Guo Q L, Qu X F, Jin W B. 2015. PhaseTank: genome-wide computational identification of phasiRNAs and their regulatory cascades. Bioinformatics 31: 284-286.
149. Chitarra W, Cuozzo D, Ferrandino A, Secchi F, Palmano S, Perrone I, Boccacci P, Pagliarani C, Gribaudo I, Mannini F et al. 2018. Dissecting interplays between Vitis vinifera L. and grapevine virus B (GVB) under field conditions. Mol Plant Pathol 19: 2651-2666.
150. Baig M S, Khan J A. 2013. Identification of Gossypium hirsutum miRNA targets in the genome of Cotton leaf curl Multan virus and Betasatellite. Indian J Biotechnol 12: 336-342.
151. Akmal M, Baig M S, Khan J A. 2017. Suppression of cotton leaf curl disease symptoms in Gossypium hirsutum through over expression of host-encoded miRNAs. J Biotechnol 263: 21-29.
152. Velten J, Cakir C, Youn E, Chen J, Cazzonelli CI. 2012. Transgene silencing and transgene-derived siRNA production in tobacco plants homozygous for an introduced AtMYB90 construct. PLoS ONE 7: e30141.
153. Zhang C-W, Liu Q, Zeng Q, Huang W-T, Wang Q, Cheng Y-Q. 2020. p24G1 encoded by Grapevine Leafroll-Associated Virus 1 suppresses RNA silencing and elicits hypersensitive response-like necrosis in Nicotiana species. Viruses 12: 1111.
154. Wesley S V, Helliwell C A, Smith N A, Wang M, Rouse D T, Liu Q, Gooding P S, Singh S P, Abbott D, Stoutjesdijk P A et al. 2001. Construct design for efficient, effective and high-throughput gene silencing in plants. Plant J 27: 581-590.
155. Pant B D, Buhtz A, Kehr J, Scheible W-R. 2008. MicroRNA399 is a long-distance signal for the regulation of plant phosphate homeostasis. Plant J 53: 731-738.
156. Chitwood D H, Nogueira F T S, Howell M D, Montgomery T A, Carrington J C, Timmermans M C P. 2009. Pattern formation via small RNA mobility. Genes Dev 23: 549-554.
157. Molnar A, Melnyk C W, Bassett A, Hardcastle T J, Dunn R, Baulcombe D C. 2010. Small silencing RNAs in plants are mobile and direct epigenetic modification in recipient cells. Science 328: 872-875.
158. Bai S, Kasai A, Yamada K, Li T, Harada T. 2011. A mobile signal transported over a long distance induces systemic transcriptional gene silencing in a grafted partner. J Exp Bot 62: 4561-4570.
159. Addo-Quaye C, Miller W, Axtell M J. 2009. CleaveLand: a pipeline for using degradome data to find cleaved small RNA targets. Bioinformatics 25: 130-131.

1. A transformed or transgenic plant that is resistant to a Grapevine Red Blotch Virus (GRBV), wherein the transformed or transgenic plant comprises:
at least one nucleic acid construct comprising:
a recombinant nucleic acid sequence encoding a suppressor of expression of a C2, a V2, or both proteins, of the GRBV,
wherein when the construct reduces the expression of the C2, V2, or both proteins in a plant that regulates transcription or expression of the C2, V2, or both proteins and confers resistance to the GRBV in the plant as compared to a control plant.

2. The plant of claim 1, wherein the plant is a grapevine.

3. The plant of claim 1, wherein at least one of:
expression of the suppressor is regulated by a constitutive, inducible, or tissue-enhanced promoter;
expression of the suppressor is regulated by a 35S promoter; or
the suppressor is a gene silencing nucleic acid that is, or is derived from, a small RNA (sRNA), microRNA (miRNA), short hairpin RNA (shRNA), bifunctional shRNA, clustered regularly interspaced palindromic repeats (CRISPR) guide RNA, or small interfering RNA (siRNA).

4. (canceled)

5. (canceled)

6. The plant of claim 1, wherein a transformed plant cell is an embryogenic cell in globular state.

7. The plant of claim 1, wherein the plant comprises one or more transformed or transgenic plant cells, and the transformed or transgenic plant cell is a grapevine cell.

8. The plant of claim 7, wherein a transformed or transgenic plant cell is a cell of one of the following grapevine varieties: 101-14Mgt, 110 Richter, 1103 Paulson, Freedom or Harmony.

9. (canceled)

10. The plant of claim 1, wherein the suppressor has at least 90, 91, 92, 93, 94, 95, 96, 97, 98, 99%, or 100% percent sequence identity with at least one of SEQ ID NOS: 3, 4, 5, 6, 7, or 8.

11. A plant part or plant material derived from the transformed or transgenic plant of claim 1.

12. A method of producing a Grapevine Red Blotch Virus (GRBV) resistant transgenic grapevine plant, wherein the method comprises introducing at least one nucleic acid construct comprising:
a recombinant nucleic acid sequence encoding a suppressor of expression of a C2, a V2, or both proteins, of the GRBV,
wherein when the construct reduces the expression of the C2, V2, or both proteins in a plant, the suppressor regulates transcription or expression of the C2, V2, or both proteins and confers resistance to GRBV in the plant as compared to a control plant.

13. A Grapevine Red Blotch Virus (GRBV) resistant grapevine plant produced by the method of claim 12.

14. A recombinant DNA vector plasmid that confers resistance against a Grapevine Red Blotch Virus (GRBV), wherein the vector plasmid contains one or more gene silencing nucleic acids against SEQ ID NO: 1, 2, or both.

15. A vector plasmid of claim 14, wherein the vector plasmid further contains a gene conferring antibiotic resistance.

16. The vector plasmid of claim 15, wherein the vector plasmid comprises a neomycin phosphotransferase II (nptII) gene conferring kanamycin resistance.

17. A transformed plant cell wherein the transformed plant cell contains and expresses one or more of the gene silencing nucleic acids in the vector plasmid of claim 14.

18. A method to confer resistance against a Grapevine Red Blotch Virus (GRBV) in non-transgenic grapevines, wherein the method comprises the steps of:
providing a group of plant cells transformed with a vector plasmid comprising:
a recombinant nucleic acid sequence encoding a suppressor of the expression of a C2, a V2, or both proteins, of the GRBV,
wherein when the construct reduces transcription or expression of the proteins C2, V2, or both proteins, and confers resistance to GRBV in the plant as compared to a control plant;
culturing the group of transformed plant cells to form transgenic seedlings resistant to the GRBV;
culturing the transgenic seedlings to take roots;
cutting an aerial part of the transgenic seedlings;
grafting a non-transgenic grapevine woody graft onto the seedling; and
culturing the graft wherein the non-transgenic grapevine plant acquires resistance against the GRBV from phloem transport of the transgenic plant.

19. The method of claim 18, wherein the suppressor is a gene silencing nucleic acid that reduces the transcription or expression of the C2, V2, or both proteins of the GRBV.

20. The method of claim 18, wherein at least one of:
the grafted non-transgenic grapevine is Vitis vinifera;
wherein the grafted non-transgenic grapevine is a variety of Vitis vinifera selected among the following table grape varieties: Autumn royal, Black seedless, Calmeria, Emperor, Flame seedless, Loose Perlette, Red Malaga, Ruby seedless, Loose Perlette, Thompson seedless, Red Globe, Sugarone and Superior seedless; or
the grafted non-transgenic grapevine is a variety of Vitis vinifera selected among the following wine grape varieties: Carmenere, Cabernet sauvignon, Cabernet Franc, Syrah, Chardonnay, Chenin, Colombard, Courdec, Daffier, Emerald, Gamay, Grenache, Malbec, Merlot, Mission, Muscat, Petit Verdot, Pinot noir, Riesling, Sauvignon, Sauvignon blanc, Semillon, Shiraz, Tempranillo, Zinfandel.

21. (canceled)

22. (canceled)

23. The method of claim 18, wherein the suppressor has at least 90, 91, 92, 93, 94, 95, 96, 97, 98, 99%, or 100% percent sequence identity with at least one of SEQ ID NOS: 3, 4, 5, 6, 7, or 8.

24. A Grapevine Red Blotch Virus (GRBV) resistant grapevine plant produced by the method of claim 18.

25. A method for producing a grapevine plant resistant to a Grapevine Red Blotch Virus (GRBV), the method comprising crossing two grapevine plants, harvesting the resultant seed or embryo and growing the seed or embryo into a mature grapevine plant, wherein at least one grapevine plant is the grapevine plant of claim 1.

26. The method of claim 25, wherein one of the grapevine plants is transgenic and the other is a non-transgenic grapevine of Vitis vinifera selected among the following table grape varieties: Autumn royal, Black seedless, Calmeria, Emperor, Flame seedless, Loose Perlette, Red Malaga, Ruby seedless, Loose Perlette, Thompson seedless, Red Globe, Sugarone and Superior seedless.

27. A grapevine plant or plant part of a transgenic grapevine cultivar, or wherein a representative sample of the plant was deposited under NCIMB No. ______.

28. A grapevine plant, or a part thereof, produced by growing or clonally propagated from the deposited sample of claim 27.

29. (canceled)

30. A tissue culture of cells produced from protoplasts or cells from the plant of claim 27, wherein the cells or protoplasts are produced from a plant part selected from the group consisting of leaf, pollen, embryo, cotyledon, hypocotyl, meristematic cell, root, root tip, pistil, anther, flowers, stem and fruit.

31. A grapevine plant regenerated from the tissue culture of claim 30, wherein the plant is resistant to the GRBV.
